(19) United States
(12) Patent Application Publication
(10) Pub. No.: US 2024/0382524 A1
(43) Pub. Date: Nov. 21, 2024

(54) TREATING MULTIPLE MYELOMA

(71) Applicant: Mayo Foundation for Medical Education and Research, Rochester, MN (US)

(72) Inventors: Latha B. Pathangey, Scottsdale, AZ (US); Peter A. Cohen, Scottsdale, AZ (US); Sandra J. Gendler, Scottsdale, AZ (US); Trupti Vardam-Kaur, Seattle, WA (US)

(21) Appl. No.: 18/691,541

(22) PCT Filed: Sep. 15, 2022
(86) PCT No.: PCT/US2022/076452

§ 371 Date: Mar. 13, 2024

Related U.S. Application Data
(60) Provisional application No. 63/245,034, filed on Sep. 16, 2021.

(51) Int. Cl. A61K 35/17; A61K 39/00; C07K 14/00; C12N 5/0783

(52) U.S. Cl. CPC A61K 35/17; A61K 39/4611; A61K 39/4644; C07K 14/001; C12N 5/0636

(57) ABSTRACT

This document relates to methods and materials related to isolated polypeptides, polypeptide preparations, and methods for using one or more isolated polypeptides to activate T cells. For example, polypeptides that can be used to activate T cells to generate antigen-specific T cells are provided. In some cases, T cells activated as described herein can be administered to a mammal having cancer (e.g., MM) or a precancerous condition (e.g., MGUS) to treat the mammal (e.g., to induce an immune response against the cancer or the precancerous condition).

### CROSS-REFERENCE TO RELATED APPLICATIONS

This application claims the benefit of U.S. Patent Application Ser. No. 63/245,034,filed on Sep. 16, 2021. The disclosure of the prior application is considered part of (and is incorporated by reference in) the disclosure of this application.

### STATEMENT REGARDING FEDERAL FUNDING

This invention was made with government support under CA186781 awarded by the National Institutes of Health. The government has certain rights in the invention.

### SEQUENCE LISTING

This application contains a Sequence Listing that has been submitted electronically as an XML file named “07039-2081WO1.XML.” The XML file, created on Aug. 15, 2022, is 464,000 bytes in size. The material in the XML file is hereby incorporated by reference in its entirety.

### TECHNICAL FIELD

This document provides methods and materials related to isolated polypeptides, polypeptide preparations, and methods for using one or more isolated polypeptides to activate T cells. For example, this document provides polypeptides that can be used to activate T cells to generate antigen-specific T cells. In some cases, T cells activated using one or more polypeptides provided herein can be administered to a mammal having cancer (e.g., multiple myeloma (MM)) or a precancerous condition (e.g., monoclonal gammopathy of undetermined significance (MGUS)) to treat the mammal (e.g., to induce an immune response against the cancer or the precancerous condition).

### BACKGROUND INFORMATION

MM is a plasma cell malignancy characterized by clonal proliferation of terminally differentiated antibody-producing plasma cells in the bone marrow, leading to osteolytic bone lesions. It is the second most common malignancy among hematological cancers with an incidence rate of 4.5-6 per 100,000 individuals per year (van de Donk et al., Lancet, 397:410-27 (2021)). The global rate of incidence and death has increased by 126% and 94%, respectively, from 1990-2016 (Cowan et al., JAMA Oncol., 4:1221-7 (2018)). Despite the availability of various therapeutic regimens, MM remains an incurable disease and patients succumb to it mainly due to development of resistance (Keats et al., Blood, 120:1067-76(2012); and Schurch et al., Virchows Arch., 476:337-51 (2020)).

Novel immunotherapies comprised of chimeric antigen receptor modified-T cells (CAR-T cells) have given encouraging results, especially in the treatment of hematological cancers. However, CAR-T cells have some limitations such as only surface antigens are targeted, poor cell persistence, exhaustion of CAR-T cells, loss of target Ag, and manufacturing difficulties (June et al., N. Engl. J. Med., 379:64-73 (2018); Shah et al., Nat. Rev. Clin. Oncol., 16:372-85 (2019)).

### SUMMARY

This document provides methods and materials relating to isolated polypeptides, polypeptide preparations, and methods for using one or more isolated polypeptides to activate T cells. For example, this document provides polypeptides (e.g., B cell maturation antigen (BCMA), mucin1 (MUC1), Fc receptor like 5 (FcRH5), myeloid cell leukemia 1 (MCL1), receptor for hyaluronan-mediated mobility (RHAMM), self-ligand receptor of the signaling lymphocytic activation molecule family 7 (SLAMF7), spliced isoform of X-box binding protein 1 (XBP(S)1), cancer testis antigen (CT45), melanoma antigen family 3/6 (MAGEA3/6), New York esophageal squamous cell carcinoma 1 (NY-ESO-1), SEPTIN9 (SEPT9), and Wilms tumor 1 (WT1) polypeptides) having the ability to be processed into different polypeptides such that the processed polypeptides as a group are capable of being presented by different major histocompatibility complex (MHC) molecules present in a particular mammalian population. In some cases, the group of processed polypeptides can bind to at least 85 percent (e.g., at least about 87, 90, or 95 percent) of the MHC molecules present in a particular mammalian population such as humans.

This document also provides methods and materials (e.g., compositions containing one or more isolated polypeptides provided herein) for treating cancer (e.g., MM) or a precancerous condition (e.g., MGUS). For example, compositions provided herein can include one or more of the BCMA, MUC1, FcRH5, MCL1, RHAMM, SLAMF7, XBP(S)1, CT45, MAGEA3/6, NY-ESO-1, SEPT9, and WT1 polypeptides provided herein (see, e.g., FIG. 1B) and can have the ability to activate T cells obtained from a mammal (e.g., a human) in culture to generate antigen-specific T cells. In some cases, a composition provided herein containing one or more of the BCMA, MUC1, FcRH5, MCL1, RHAMM, SLAMF7, XBP(S)1, CT45, MAGEA3/6, NY-ESO-1, SEPT9, and WT1 polypeptides provided herein can be used in vitro to activate T cells obtained from a mammal (e.g., a human) to generate antigen-specific T cells, and those antigen-specific T cells can be re-infused into that mammal to treat cancer (e.g., MM) within that mammal. In some cases, a composition provided herein containing one or more of the BCMA, MUC1, FcRH5, MCL1, RHAMM, SLAMF7, XBP(S)1, CT45, MAGEA3/6, NY-ESO-1, SEPT9, and WT1 polypeptides provided herein can be administered to a mammal (e.g., a human) to activate T cells within the mammal to generate antigen-specific T cells that can reduce the number of cancer cells (e.g., MM cells) within that mammal.

As described herein, long polypeptides (e.g., ranging from about 17-41 amino acid residues in length) were identified as having the ability to generate antigen-specific CD4⁺ T cells and/or antigen-specific CD8⁺ T cells (e.g., antigen-specific CD4⁺ and/or antigen-specific CD8⁺ effector memory (T_{EM}) cells and/or antigen-specific CD4⁺ and/or antigen-specific CD8⁺ central memory (T_{CM}) cells) from peripheral blood mononuclear cells (PBMCs). Also as described herein, antigen-specific T_{EM} cells and T_{CM} cells generated as described herein can induce an immune response against cancer cells and/or precancerous cells expressing one or more of the polypeptides. Having the ability to generate antigen-specific CD4⁺ T cells and/or antigen-specific CD8⁺ T cells that can induce immune responses against a particular cancer using selected polypeptides expressed by that cancer can enable the development of cancer treatments that are targeted, inexpensive, and can be rapidly produced.

In general, one aspect of this document features an isolated polypeptide consisting of the amino acid sequence set forth in any one of SEQ ID NOs: 1-364. The isolated polypeptide can consist of the amino acid sequence set forth in any one of SEQ ID NOs: 1-33.

In another aspect, this document features a composition comprising an isolated polypeptide consisting of the amino acid sequence set forth in any one of SEQ ID NOs: 1-364. The polypeptide can consist of the amino acid sequence set forth in any one of SEQ ID NOs: 1-33. The composition can comprise a polypeptide consisting of the amino acid sequence set forth in SEQ ID NO: 1, a polypeptide consisting of the amino acid sequence set forth in SEQ ID NO:2, and a polypeptide consisting of the amino acid sequence set forth in SEQ ID NO:3. The composition can comprise a polypeptide consisting of the amino acid sequence set forth in SEQ ID NO:1, a polypeptide consisting of the amino acid sequence set forth in SEQ ID NO:20, a polypeptide consisting of the amino acid sequence set forth in SEQ ID NO: 23, a polypeptide consisting of the amino acid sequence set forth in SEQ ID NO:24, and a polypeptide consisting of the amino acid sequence set forth in SEQ ID NO:31. The composition can comprise a polypeptide consisting of the amino acid sequence set forth in SEQ ID NO:4, a polypeptide consisting of the amino acid sequence set forth in SEQ ID NO: 7, a polypeptide consisting of the amino acid sequence set forth in SEQ ID NO:8, a polypeptide consisting of the amino acid sequence set forth in SEQ ID NO: 13, and a polypeptide consisting of the amino acid sequence set forth in SEQ ID NO:14. The composition can comprise a polypeptide consisting of the amino acid sequence set forth in SEQ ID NO: 10, a polypeptide consisting of the amino acid sequence set forth in SEQ ID NO: 11, a polypeptide consisting of the amino acid sequence set forth in SEQ ID NO:12, a polypeptide consisting of the amino acid sequence set forth in SEQ ID NO:28, and a polypeptide consisting of the amino acid sequence set forth in SEQ ID NO:29. The composition can comprise a polypeptide consisting of the amino acid sequence set forth in SEQ ID NO: 1, a polypeptide consisting of the amino acid sequence set forth in SEQ ID NO: 2, a polypeptide consisting of the amino acid sequence set forth in SEQ ID NO:3, a polypeptide consisting of the amino acid sequence set forth in SEQ ID NO:4, a polypeptide consisting of the amino acid sequence set forth in SEQ ID NO:10, a polypeptide consisting of the amino acid sequence set forth in SEQ ID NO: 12, a polypeptide consisting of the amino acid sequence set forth in SEQ ID NO:13, a polypeptide consisting of the amino acid sequence set forth in SEQ ID NO:20, a polypeptide consisting of the amino acid sequence set forth in SEQ ID NO:28, a polypeptide consisting of the amino acid sequence set forth in SEQ ID NO: 29 and a polypeptide consisting of the amino acid sequence set forth in SEQ ID NO: 31. The composition can comprise a polypeptide consisting of the amino acid sequence set forth in SEQ ID NO:1, a polypeptide consisting of the amino acid sequence set forth in SEQ ID NO:2, a polypeptide consisting of the amino acid sequence set forth in SEQ ID NO: 3, a polypeptide consisting of the amino acid sequence set forth in SEQ ID NO:4, a polypeptide consisting of the amino acid sequence set forth in SEQ ID NO: 10, a polypeptide consisting of the amino acid sequence set forth in SEQ ID NO: 12, a polypeptide consisting of the amino acid sequence set forth in SEQ ID NO: 13, a polypeptide consisting of the amino acid sequence set forth in SEQ ID NO:20, a polypeptide consisting of the amino acid sequence set forth in SEQ ID NO:23, a polypeptide consisting of the amino acid sequence set forth in SEQ ID NO:28, a polypeptide consisting of the amino acid sequence set forth in SEQ ID NO: 29, and a polypeptide consisting of the amino acid sequence set forth in SEQ ID NO: 32. The composition can comprise a polypeptide consisting of the amino acid sequence set forth in SEQ ID NO:2, a polypeptide consisting of the amino acid sequence set forth in SEQ ID NO:4, a polypeptide consisting of the amino acid sequence set forth in SEQ ID NO: 12, a polypeptide consisting of the amino acid sequence set forth in SEQ ID NO:13, and a polypeptide consisting of the amino acid sequence set forth in SEQ ID NO:29. The composition can comprise a polypeptide consisting of the amino acid sequence set forth in SEQ ID NO: 1, a polypeptide consisting of the amino acid sequence set forth in SEQ ID NO: 10, and a polypeptide consisting of the amino acid sequence set forth in SEQ ID NO:20. The composition can comprise a polypeptide consisting of the amino acid sequence set forth in SEQ ID NO:2, a polypeptide consisting of the amino acid sequence set forth in SEQ ID NO: 4, and a polypeptide consisting of the amino acid sequence set forth in SEQ ID NO:29. The composition can comprise a polypeptide consisting of the amino acid sequence set forth in SEQ ID NO:3, a polypeptide consisting of the amino acid sequence set forth in SEQ ID NO: 12, and a polypeptide consisting of the amino acid sequence set forth in SEQ ID NO: 13. The composition can comprise a polypeptide consisting of the amino acid sequence set forth in SEQ ID NO:11, a polypeptide consisting of the amino acid sequence set forth in SEQ ID NO: 14, and a polypeptide consisting of the amino acid sequence set forth in SEQ ID NO:28. The composition can comprise a polypeptide consisting of the amino acid sequence set forth in SEQ ID NO:28 and a polypeptide consisting of the amino acid sequence set forth in SEQ ID NO: 29. The composition can comprise a polypeptide consisting of the amino acid sequence set forth in SEQ ID NO:13 and a polypeptide consisting of the amino acid sequence set forth in SEQ ID NO:14. The composition can comprise a polypeptide consisting of the amino acid sequence set forth in SEQ ID NO: 19 and a polypeptide consisting of the amino acid sequence set forth in SEQ ID NO:20. The composition can comprise a polypeptide consisting of the amino acid sequence set forth in SEQ ID NO: 10, a polypeptide consisting of the amino acid sequence set forth in SEQ ID NO:11, and a polypeptide consisting of the amino acid sequence set forth in SEQ ID NO:12. The composition can comprise at least five polypeptides consisting of the amino acid sequence set forth in any of SEQ ID NOs: 1-33. The composition can comprise at least ten polypeptides consisting of the amino acid sequence set forth in any of SEQ ID NOs: 1-33. The composition can comprise at least 11 polypeptides consisting of the amino acid sequence set forth in any of SEQ ID NOs: 1-33. The composition can comprise at least 12 polypeptides consisting of the amino acid sequence set forth in any of SEQ ID NOs: 1-33.

In another aspect, this document features a composition comprising at least two polypeptides, wherein each of the at least two polypeptides is a polypeptide consisting of the amino acid sequence set forth in any one of SEQ ID NOs: 1-364. Each of the at least two polypeptides can consist of the amino acid sequence set forth in any one of SEQ ID NOs: 1-33. The composition can comprise a polypeptide consisting of the amino acid sequence set forth in SEQ ID NO: 1, a polypeptide consisting of the amino acid sequence set forth in SEQ ID NO: 2, and a polypeptide consisting of the amino acid sequence set forth in SEQ ID NO:3. The composition can comprise a polypeptide consisting of the amino acid sequence set forth in SEQ ID NO:1, a polypeptide consisting of the amino acid sequence set forth in SEQ ID NO: 20, a polypeptide consisting of the amino acid sequence set forth in SEQ ID NO:23, a polypeptide consisting of the amino acid sequence set forth in SEQ ID NO:24, and a polypeptide consisting of the amino acid sequence set forth in SEQ ID NO:31. The composition can comprise a polypeptide consisting of the amino acid sequence set forth in SEQ ID NO:4, a polypeptide consisting of the amino acid sequence set forth in SEQ ID NO: 7, a polypeptide consisting of the amino acid sequence set forth in SEQ ID NO:8, a polypeptide consisting of the amino acid sequence set forth in SEQ ID NO:13, and a polypeptide consisting of the amino acid sequence set forth in SEQ ID NO:14. The composition can comprise a polypeptide consisting of the amino acid sequence set forth in SEQ ID NO:10, a polypeptide consisting of the amino acid sequence set forth in SEQ ID NO: 11, a polypeptide consisting of the amino acid sequence set forth in SEQ ID NO: 12, a polypeptide consisting of the amino acid sequence set forth in SEQ ID NO:28, and a polypeptide consisting of the amino acid sequence set forth in SEQ ID NO:29. The composition can comprise a polypeptide consisting of the amino acid sequence set forth in SEQ ID NO:1, a polypeptide consisting of the amino acid sequence set forth in SEQ ID NO: 2, a polypeptide consisting of the amino acid sequence set forth in SEQ ID NO:3, a polypeptide consisting of the amino acid sequence set forth in SEQ ID NO:4, a polypeptide consisting of the amino acid sequence set forth in SEQ ID NO: 10, a polypeptide consisting of the amino acid sequence set forth in SEQ ID NO: 12, a polypeptide consisting of the amino acid sequence set forth in SEQ ID NO: 13, a polypeptide consisting of the amino acid sequence set forth in SEQ ID NO:20, a polypeptide consisting of the amino acid sequence set forth in SEQ ID NO:28, a polypeptide consisting of the amino acid sequence set forth in SEQ ID NO: 29 and a polypeptide consisting of the amino acid sequence set forth in SEQ ID NO: 31. The composition can comprise a polypeptide consisting of the amino acid sequence set forth in SEQ ID NO:1, a polypeptide consisting of the amino acid sequence set forth in SEQ ID NO:2, a polypeptide consisting of the amino acid sequence set forth in SEQ ID NO: 3, a polypeptide consisting of the amino acid sequence set forth in SEQ ID NO:4, a polypeptide consisting of the amino acid sequence set forth in SEQ ID NO: 10, a polypeptide consisting of the amino acid sequence set forth in SEQ ID NO: 12, a polypeptide consisting of the amino acid sequence set forth in SEQ ID NO: 13, a polypeptide consisting of the amino acid sequence set forth in SEQ ID NO:20, a polypeptide consisting of the amino acid sequence set forth in SEQ ID NO:23, a polypeptide consisting of the amino acid sequence set forth in SEQ ID NO:28, a polypeptide consisting of the amino acid sequence set forth in SEQ ID NO: 29, and a polypeptide consisting of the amino acid sequence set forth in SEQ ID NO: 32. The composition can comprise a polypeptide consisting of the amino acid sequence set forth in SEQ ID NO:2, a polypeptide consisting of the amino acid sequence set forth in SEQ ID NO:4, a polypeptide consisting of the amino acid sequence set forth in SEQ ID NO: 12, a polypeptide consisting of the amino acid sequence set forth in SEQ ID NO:13, and a polypeptide consisting of the amino acid sequence set forth in SEQ ID NO:29. The composition can comprise a polypeptide consisting of the amino acid sequence set forth in SEQ ID NO: 1, a polypeptide consisting of the amino acid sequence set forth in SEQ ID NO: 10, and a polypeptide consisting of the amino acid sequence set forth in SEQ ID NO:20. The composition can comprise a polypeptide consisting of the amino acid sequence set forth in SEQ ID NO:2, a polypeptide consisting of the amino acid sequence set forth in SEQ ID NO: 4, and a polypeptide consisting of the amino acid sequence set forth in SEQ ID NO:29. The composition can comprise a polypeptide consisting of the amino acid sequence set forth in SEQ ID NO:3, a polypeptide consisting of the amino acid sequence set forth in SEQ ID NO: 12, and a polypeptide consisting of the amino acid sequence set forth in SEQ ID NO: 13. The composition can comprise a polypeptide consisting of the amino acid sequence set forth in SEQ ID NO:11, a polypeptide consisting of the amino acid sequence set forth in SEQ ID NO: 14, and a polypeptide consisting of the amino acid sequence set forth in SEQ ID NO:28. The composition can comprise a polypeptide consisting of the amino acid sequence set forth in SEQ ID NO:28 and a polypeptide consisting of the amino acid sequence set forth in SEQ ID NO: 29. The composition can comprise a polypeptide consisting of the amino acid sequence set forth in SEQ ID NO:13 and a polypeptide consisting of the amino acid sequence set forth in SEQ ID NO:14. The composition can comprise a polypeptide consisting of the amino acid sequence set forth in SEQ ID NO:19 and a polypeptide consisting of the amino acid sequence set forth in SEQ ID NO:20. The composition can comprise a polypeptide consisting of the amino acid sequence set forth in SEQ ID NO: 10, a polypeptide consisting of the amino acid sequence set forth in SEQ ID NO: 11, and a polypeptide consisting of the amino acid sequence set forth in SEQ ID NO:12. The composition can comprise at least five polypeptides consisting of the amino acid sequence set forth in any of SEQ ID NOs: 1-33. The composition can comprise at least ten polypeptides consisting of the amino acid sequence set forth in any of SEQ ID NOs: 1-33. The composition can comprise at least 11 polypeptides consisting of the amino acid sequence set forth in any of SEQ ID NOs: 1-33. The composition can comprise at least 12 polypeptides consisting of the amino acid sequence set forth in any of SEQ ID NOs: 1-33.

In another aspect, this document features a method for activating T cells having specificity for a cancer antigen. The method comprises (or consists essentially of, or consists of) contacting a cell population comprising T cells with at least one polypeptide consisting of the amino acid sequence set forth in any one of SEQ ID NOs: 1-364. The cell population can comprise unfractionated PBMCs. The cells of the cell population can be human cells. The contacting can be performed in vitro.

In another aspect, this document features a method of treating a mammal having cancer or a precancerous condition. The method comprises (or consists essentially of, or consists of) contacting T cells with at least one polypeptide consisting of the amino acid sequence set forth in any one of SEQ ID NOs: 1-364 to activate the T cells, and administering the activated T cells to the mammal. The mammal can be a human. The T cell can be T cells obtained from the mammal. The mammal can have the cancer, and the administering can reduce the number of cancer cells within the mammal. The cancer can be selected from the group consisting of MM, colorectal cancer, breast cancer, non-Hodgkin's lymphoma, and ovary cancer. The mammal can have the precancerous condition, and the administering can reduce a symptom of the precancerous condition within the mammal. The precancerous condition can be MGUS. The method can further comprise expanding the activated T-cells prior to administering the activated T-cells to the mammal.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1B. Synthetic polypeptides were designed for different antigens based on predictive algorithms. FIG. 1A) An example depicting the methodology used to design polypeptides. Immunogenic heat map that recognizes regions with high binding affinity for MHC I (bold and italicized, first line under the amino acid) and MHC II (bold, second line under the amino acid) grooves for antigen, CT45 (SEQ ID NO: 365; brackets indicate the designed polypeptide sequence). FIG. 1B) The polypeptide sequences for different antigens (17-41 mers) were synthesized that consist of overlapping regions for MHC I and MHC II binding. The polypeptides: 1-18 are designed from antigens that are overexpressed in MM; polypeptides 19 through 33 were constructed from cancer testis antigens. The list consists of antigens (MUC1 (SEA1, 2, and 3), CD38, FcRH5, RHAMM, SLAMF7, SOX2, XBP(S)1, CT45, MAGEA6, MAGEC1, and NY-ESO-1) that showed more than one region with overlapping MHC I & II hotspots that could be synthesized. To indicate that the polypeptides are synthesized from the same antigen, the polypeptides are labelled accordingly (e.g., CD38.1, CD38.2).

FIGS. 2A-2F. Natural CD4⁺ and CD8⁺ T cells from unfractionated healthy donor PBMCs are activated and readily propagated by polypeptides in an Ag-specific manner. Freshly thawed PBMCs from healthy donors were exposed to single polypeptides (50 μg/ml) in the presence of GM-CSF and Toll-like receptor agonists (resiquimod and LPS) followed by γ_{c} cytokine IL-7. Representative dot-plots for CD4⁺IFN-γ⁺ (top panel) and CD8⁺IFN-γ⁺ (bottom panel) following secondary stimulation of T cells generated against (FIG. 2A) SEA1 (designed from MUC1 Ag), (FIG. 2B) RHAMM2 and (FIG. 2C) MCL1.1 with either unpulsed or the specific polypeptide-pulsed PBMCs. Values shown for unpulsed cells are typical and have been subtracted in subsequent figures. (FIG. 2D) Bar graph depicting the percentage of Ag-specific and Ag non-specific CD4⁺IFN-γ⁺ and (FIG. 2E) CD8⁺IFN-γ⁺ T cells. (FIG. 2F) Graph depicting percentages of CD4⁺ and CD8⁺ T cells and fold expansion (triangles) observed for T cells generated following primary stimulation with SEA1, SLAMF7.5, MCL1.1, RHAMM2, RHAMM3, RHAMM4, WT1.1, XBP(S)1.1, XBP(S)1.2 or BCMA2. Data from two experiments.

FIG. 3. Four polypeptide cocktails were used for subsequent experiments. Based on the data obtained following treatment of healthy donor PBMCs with single polypeptides, four different polypeptide cocktails were designed to assess the ability of different antigens to co-operatively induce T cell responses from PBMCs isolated from healthy donors or MM patients.

FIGS. 4A-4C. PBMCs from healthy donors or multiple myeloma patients generated Ag-specific T cells following stimulation with four different polypeptide cocktails designed from various antigens. PBMCs from healthy donors (HD) or multiple myeloma (MM) patients' bloods (100 mL) were stimulated with 4 different cocktails, each consisting of either 3 or 5 polypeptides at 25 μg/mL for each polypeptide. Cells were harvested on day 19. Shown are percentages of (FIG. 4A) Ag-specific CD4⁺IFN-γ⁺+CD8⁺IFN-γ⁺ and (FIG. 4B) CD4⁺IFN-γ⁺ and (FIG. 4C) CD8⁺IFN-γ⁺ T cells for HDs and MM patients observed following secondary stimulation with PBMCs pulsed with specific polypeptides present in MUC1 cocktail, cocktail 1, cocktail 3 and cocktail 4 at the end of the culture period (D19). Cocktails 3 and 4 lack MM2 and MM5 due to unavailability of cells. No statistically significant differences (NS) were observed between the different cocktails or between the HDs and MM patients in each cocktail (Student's t-test p>0.1 in every comparison).

FIGS. 5A-5C. Stimulation with polypeptide cocktails enriches T cells equivalently regardless of the disease status. FIG. 5A. Depiction of percentages of CD4⁺ (black) and CD8⁺ (grey) T cells at the end of culture period for 5 HDs (left panel) and 5 MM patients (right panel). FIG. 5B. Pie charts showing percentages of immune cell subsets on DO or D19 at end of culture period of PBMCs of HD (left panel) and MM patient (right panel) with MUC1 cocktail and cocktails 1, 3, and 4. CD19, CD56, CD33 and CD3 are shown. CD3⁺ population on D19 was always greater than 85% positive. The numbers shown in the quadrants represent the percentages. FIG. 5C. CD3⁺ T cells were further analyzed for CD4⁺, CD8⁺ and CD56⁺ for HD (left panel) and MM patient (right panel). Percents of CD4⁺ and CD8⁺ T cells depended upon the cocktail used for primary stimulation and the HLA genotype of the individual. Data were similar for all ten samples. No statistically significant differences were observed (Student's t-test). Representative data are shown.

FIGS. 6A-6C. Generation of both effector and memory T cells in MM patients and HDs following polypeptide activation. Flow cytometry dot plots depicting (FIG. 6A) CD4⁺ and (FIG. 6B) CD8⁺ T_{EM} (CD45RO⁺CD62⁻) and T_{CM} (CD45RO⁺CD62⁺) for HD and MM on D19 following stimulation of multipeptide cocktails. Representative data shown for 2 individuals. FIG. 6C. Chart showing composite results of T_{EM} and T_{CM} for MUC1-activated HD (top) and MM patients (bottom). Statistical analysis indicated no significant differences (Student's t-test).

FIGS. 7A-7C. Culture activation generates T_{EM} and T_{CM} populations in both CD4+ and CD8⁺ T cells. Table depicting the percentage of T_{EM} and T_{CM} for CD4+ and CD8+ T cells obtained at the end of the culture period following treatment with cocktails (FIG. 7A) CT1, (FIG. 7B) CT3, and (FIG. 7C) CT4.

FIGS. 8A-8D. Stimulation with polypeptide cocktail leads to enhanced expression of TRM markers, CD69 and CD103, on CD4⁺ and CD8⁺ T cells. (FIG. 8A) Expression of CD69 and CD103 on CD4⁺ T cells or (FIG. 8B) CD8⁺ T cells on D0 or on D19 following stimulation of PBMCs isolated from healthy donor (HD) or MM patient (MM) with either MUC1 Cocktail, Cocktail 1, Cocktail 3 or Cocktail 4. Representative data are shown. CD122 expression on CD4⁺ T cells (FIG. 8C) and CD8⁺ T cells (FIG. 8D) was generated following exposure of PBMCs from HD (top panel) and MM patient (bottom panel) to MUC1 cocktail (solid line histogram), Cocktail 1 (dotted line histogram), and Cocktail 4 (dashed line histogram). The isotype control is depicted by grey histogram. Data representative of four individuals (2 HDs, 2 MM patients).

FIG. 9. Effector memory (T_{EM}) and central memory (T_{CM}) CD4⁺ and CD8⁺ T cells possess anti-tumor profile. Representative dot plot showing the gating hierarchy to define different functional subsets of CD4⁺ and CD8⁺ T cells. First, viable cells were gated based on the absence of UV Blue stain. These cells are then gated on CD3 and then on CD8, which was used to define CD8⁺IFN-γ⁺. The expression of perforin and granzyme B was examined on CD8⁺IFN-γ⁺. Similar strategy was used for CD4⁺ T cells. Representative data are shown.

FIGS. 10A-10C. Functional characterization following stimulation to polypeptide cocktails leads to multiclonal expansion of Ag-specific CD4⁺ and CD8⁺ T cells possessing cytolytic capabilities at the end of the culture period (D19). Dot-plot showing expression of perforin and granzyme B on (FIG. 10A) CD8⁺ T cells in HD1 (top panel) and MM1 (bottom panel) following restimulation with each polypeptide from CT-3. T cell receptor (TCR) Vβ repertoire based on flow analysis of (FIG. 10B) CD3⁺CD4⁺ and (FIG. 10C) CD3⁺CD8⁺ of HD1 or MM1 harvested on day 19. Representative data are shown and statistical analysis indicated no significant differences among the ten samples analyzed (Student's t-test).

FIGS. 11A-11D. Metabolic profile of healthy donor or multiple myeloma patient's memory T cell population varies depending upon the polypeptide cocktail used for stimulation. Glycolysis stress test was conducted to examine the extracellular acidification rate (ECAR) in response to glucose, oligomycin, and 2-deoxy-D-glucose (2DG). The Mito-cell stress test assessed the oxygen consumption rate (OCR) following treatment with oligomycin, carbonyl cyanide-p-trifluoromethoxyphenylhydrazone (FCCP) and rotenone/antimycin. The histograms for ECAR (left panel) and OCR (right panel) are depicted for T cells generated following exposure of PBMCs from healthy donor (dotted lines) or multiple myeloma patient (solid lines) to MUC1 cocktail (FIG. 11A), cocktail 1 (FIG. 11B), cocktail 3 (FIG. 11C), and cocktail 4 (FIG. 11D). Representative data are shown, and no statistically significant differences were observed among the six samples analyzed (Student's t-test).

FIGS. 12A-12F. Ag-specific T cells generated following stimulation with a cocktail containing ten different peptide designed from various antigens. As described previously in FIG. 4, PBMCs from healthy donors (HD) were stimulated with a peptide cocktail containing 10 different polypeptides at 10 μg/mL for each polypeptide. Cells were harvested on day 19. Shown are percentages of (FIG. 12A) Ag-specific CD4⁺IFN-γ⁺+CD8⁺IFN-γ⁺ and (FIG. 12B) CD4⁺IFN-γ⁺ and (FIG. 12C) CD8⁺IFN-γ⁺ T cells for HDs observed following secondary stimulation with PBMCs pulsed with single polypeptides present in the cocktail at the end of the culture period (D19). (FIG. 12D) Percentage of CD4⁺CD8⁺IFN-γ⁺ T cells following re-exposure of MM6 PBMC-derived T cells to single peptides from the cocktail employed for primary stimulation. To test the effect of peptide concentration on the 10-peptide cocktails, PBMCs from HD5 were stimulated with peptides using two concnetrations, 5 μg/mL and 10 μg/mL for each peptide in Ag-specific CD4⁺IFN-γ⁺ (FIG. 12E) and CD8⁺IFN-γ⁺ T (FIG. 12F) cells. The lower peptide concentration appears to provide a stronger stimulation.

### DETAILED DESCRIPTION

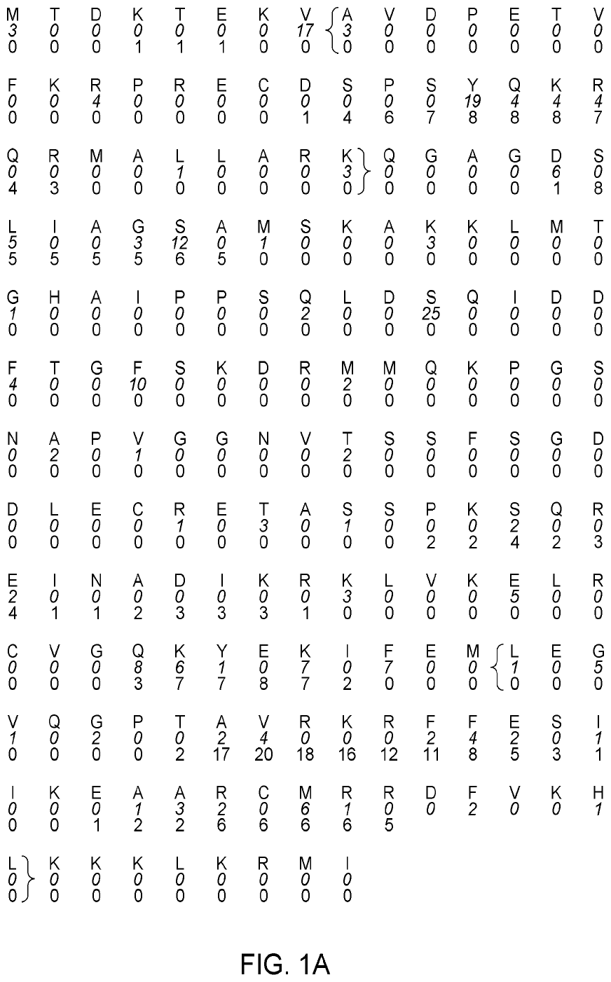

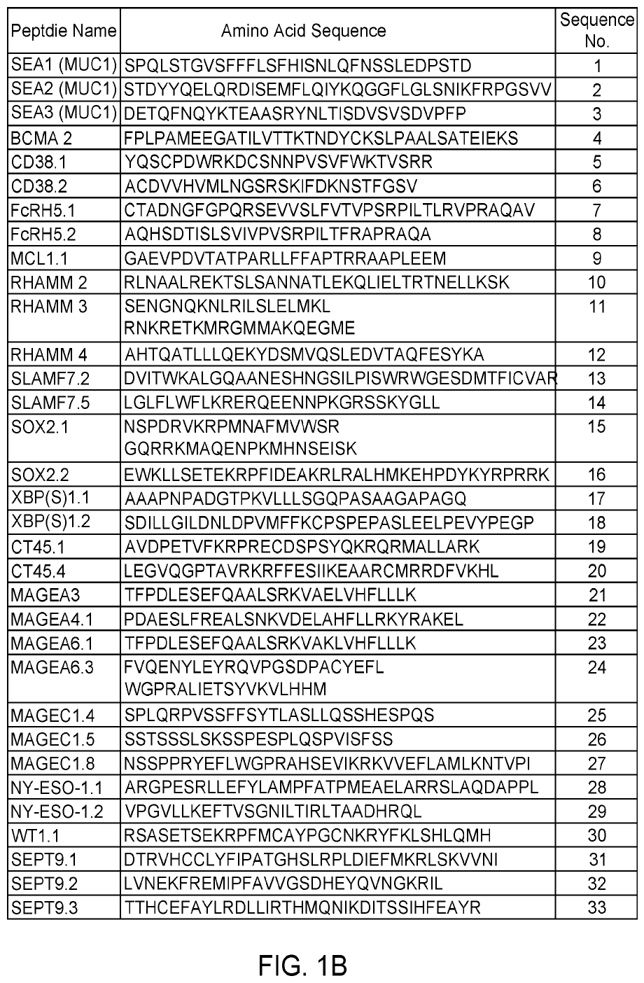

This document provides isolated polypeptides, polypeptide preparations, and methods for using one or more isolated polypeptides to activate T cells. For example, this document provides polypeptides that have the ability to be naturally processed and presented by different MHC molecules. In some cases, an isolated polypeptide provided herein can have a sequence present in a polypeptide having an elevated level of expression in a cancer (e.g., MM) and/or a precancerous condition (e.g., MGUS). For example, this document provides the isolated polypeptides set forth in FIG. 1B and Tables 1-33. In some cases, an isolated polypeptide provided herein can be a substantially pure polypeptide that comprises, consists essentially of, or consists of the amino acid sequence set forth in any one of SEQ ID NOs: 1-33. The term “isolated” refers to material which is substantially or essentially free from components that normally accompany the material as it is found in its native state. Thus, isolated polypeptides as described in this document do not contain at least some of the materials normally associated with the polypeptides in their in situ environment. The term “polypeptide” refers to a chain of amino acids linked by peptide bonds.

A polypeptide provided herein can be any appropriate length (e.g., can include any appropriate number of amino acids). In some cases, a polypeptide provided herein can be a fragment of a full-length polypeptide. For example, a polypeptide provided herein can be longer than 17 amino acid residues in length and shorter than the corresponding fulllength polypeptide. For example, a polypeptide provided herein can be from about 17 amino acids to about 50 amino acids (e.g., from about 17 to about 40 amino acids, from about 17 to about 35 amino acids, from about 17 to about 30 amino acids, from about 17 to about 25 amino acids, or from about 17 to about 20 amino acids) in length.

A polypeptide provided herein can be derived from any appropriate polypeptide. In some cases, a polypeptide provided herein can be derived from (e.g., can be a fragment of) a cancer antigen polypeptide (e.g., a tumor specific antigen polypeptide or a tumor associated antigen polypeptide). Examples of polypeptides from which a polypeptide provided herein can be derived from include, without limitation, BCMA polypeptides, MUC1 polypeptides, FcRH5 polypeptides, MCL1 polypeptides, RHAMM polypeptides, SLAMF7 polypeptides, XBP(S)1 polypeptides, CT45 polypeptides, MAGEA3/6 polypeptides, NY-ESO-1 polypeptides, SEPT9 polypeptides, and WT1 polypeptides.

A polypeptide provided herein can include any appropriate sequence. In some cases, a polypeptide provided herein can have a sequence present in a cancer antigen polypeptide such as a BCMA, MUC1, FcRH5, MCL1, RHAMM, SLAMF7, XBP(S)1, CT45, MAGEA3/6, NY-ESO-1, SEPT9, or WT1 polypeptide. In some cases, a polypeptide provided herein can comprise, consist essentially of, or consist of an amino acid sequence set forth in FIG. 1B.

In some cases, a polypeptide provided herein can be a variant polypeptide that consists of the amino acid sequence set forth in any one of SEQ ID NOs: 1-33 except that the variant polypeptide includes one, two, three, four, or five amino acid substitutions within the articulated sequence of the sequence identifier (e.g., SEQ ID NO:1), has one, two, three, four, or five amino acid residues preceding the articulated sequence of the sequence identifier (e.g., SEQ ID NO:1), and/or has one, two, three, four, or five amino acid residues following the articulated sequence of the sequence identifier (e.g., SEQ ID NO:1), provided that the polypeptide has the ability to be naturally processed and presented by different MHC molecules. Examples of such variant polypeptides for SEQ ID NOs: 1-33 are set forth in Tables 1-33, respectively.

TABLE 1
Examples of variant polypeptides of SEQ ID NO: 1.

| Variant Polypeptide Sequence | SEQ ID NO: |
|---|---|
| QLSTGVSFFFLSFHISNLQFNSSLEDPSTD | 34 |
| SPQLSTGVSFFFLSFHISNLQFNSSLED | 35 |
| SPQLSTGVSFFFLTFHLSNLNFNSSLEDPST | 36 |
| SVQISTGVSFFFLSFHISNLQFNSSLEDPSTD | 37 |
| PQLYTGVSFFFLSFHISNLQFNSSLEDPSTD | 38 |
| SPQLSTGVSFFMLSFHISNLQFNPSLEDPSTD | 39 |
| SPQLSTGVSFFFLSFHISNLQFNSSLADPSTD | 40 |
| SPVQLSTGVSFFFLSFHISNLQFNPSLEDPST | 41 |
| SPQLSTGVSFFFLYFHISNLQFNSSLEDPSTD | 42 |
| SPQLSTGVSFFFLTFHISNLQFNSSLPDTSTD | 43 |
| PQFSTGVSFFFLTFHISNLQFNSSLADPSTD | 44 |

TABLE 2
Examples of variant polypeptides of SEQ ID NO: 2.

| | SEQ |
|---|---|
| Variant Polypeptide Sequence | ID NO: |
| STDYYQELQRDISEMFLQIYKQGGFLGLSNIKFRPGS | 45 |
| STDYYQALQRDISEMFLQIYKQGGFLGLSNIKFRPGSVV | 46 |
| STSDYYQELQRDISNMFLQIYKQGGMLGLSNIKFRIGSVA | 47 |
| STDYYQALQRDISEMFLQIYKQGGFLGLSNIKFRPGSVV | 48 |
| STDYYQELQRDISEMYLQIYKQTGFLGLSNIKFRPGSVV | 49 |
| STDYYQELQRDISYMFLQIYKQGGFLGLSNIKMRPGTVV | 50 |
| DYYQELQRDISEMFLQIYKQGGFLGLSNIKFRPGSVV | 51 |
| STDYYQELQRDISEMFLFIYKQGGFLGLSNIKFRPGSVV | 52 |
| STDYYQELQRDISEMFLQIYKQGMFLGLSNIKFRPGSVV | 53 |
| STEYAQELQRDISEMFLQIYKQGGFLGLSNIKFRPGSVV | 54 |

TABLE 3
Examples of variant polypeptides of SEQ ID NO: 3.

| Variant Polypeptide Sequence | SEQ ID NO: |
|---|---|
| TQFNQYKTEAASRYNLTISDVSVSDVP | 55 |
| DVATQMNQYKTEAASNYNLTISDVSVSDVPFP | 56 |
| DVETQFNQYKTEAASRYNLTISDVSVSDV | 57 |
| DVETQFNQYKTEAASRYNLTISDVSVSDAPFP | 58 |
| DVETQFNQYKTEAASRYNLTISDLSPSDLPFP | 59 |
| DVATQFNQYKTEAPSRYNLTISDVSLSDVPFP | 60 |
| DVETQMNQYKTEAASRYNLTISDVSVSDVPFP | 61 |
| DVYTQFNQYKTEAASRYNLSISDVSVSDVPFP | 62 |
| DVETQFNQYKTEAASRYNLTISDVSVSDVPFP | 63 |
| DVETQFNQYKTAAASRYNLVTISDVSVSDVPFP | 64 |

TABLE 4
Examples of variant polypeptides of SEQ ID NO: 4.

| Variant Polypeptide Sequence | SEQ ID NO: |
|---|---|
| LPAMEEGATILVTTKTNDYCKSLPAALSATEI | 65 |
| FPLPAMEEGATILVTTKTNDYCKSLPAALSAT | 66 |
| YPLPAMEAGATILVTTKTNDMCKSLPAALSATEIEKS | 67 |
| FPLPAMEEGATILVTTKINDQCKSLPAALSATEIEKS | 68 |
| FPLPAMEEGATILVTTKTNDYYKSLPAALSATEIEKS | 69 |
| FPLPAMEEGATILVTTKTNDYCKSLPATSTATEIE | 70 |
| PAMEEGATILVTTKTNDYCKSLPAVLSATNIEKS | 71 |
| FPLPAMENGATIVVTTKTNDMCKSLPAALSATEIEKS | 72 |
| FPLPAMEEGATILVTTKTNDYCKSLPAALSATDIAKS | 73 |
| FPLPAMEPGATILVTTKTNYYCKSLPAALSATEIEKS | 74 |

TABLE 5
Examples of variant polypeptides of
SEQ ID NO: 5.

| | SEQ ID |
|---|---|
| Variant Polypeptide Sequence | NO: |
| PDWRKDCSNNPVSVFWKTVSRR | 75 |
| YQSCPDWRKDCSNNPVSVFWKTV | 76 |
| YQSTCPDWRKDCSNNPVSVFWVTVSRR | 77 |
| YQSCPDWRKDQSNQPVSVMWKSVSKR | 78 |
| YQSCPDWRKDQSNNPVSVFWKTVSRR | 79 |
| YQSCPDWRKDNSNNPVSVFWKTVSRR | 80 |
| YQSCPDWRKDCSNNPVSVFFKTVSRR | 81 |
| YQSCPDWRKDYSNNPVSVFWKTVSR | 82 |
| YQSCPDTRKDCSNNPVSVFWKTVSRR | 83 |
| YQSQPDWRKDCSNNPVSVFWKSVSRR | 84 |

TABLE 6
Examples of variant polypeptides
of SEQ ID NO: 6.

| | SEQ ID |
|---|---|
| Variant Polypeptide Sequence | NO: |
| ACDVVHVFLNGSRSKIFDKNSTFGSV | 85 |
| ACDVVHVMLNGSRSKIFDKNST | 86 |
| AFDVVHVMLNGSRSKIFDKNSTFGSE | 87 |
| ACDVIHVMLNGSRTKIFDKNSSFGSV | 88 |
| AQDVVHVMPNGTRSKLFDKNSTFGSV | 89 |
| APDVVHVMVNGSRSKIFDKNSTFGSV | 90 |
| ACDVVHVMLNGSRSPKIWDKNSTFGSV | 91 |
| ACDVVHVMLNGSRSKIQDKNSTFGTV | 92 |
| ACDVVHVMLNGSRSKIFDKNSTFFSV | 93 |
| ACDVVHNMLNGSRSKIFDKNSTFGSV | 94 |

TABLE 7
Examples of variant polypeptides
of SEQ ID NO: 7.

| | SEQ |
|---|---|
| | ID |
| Variant Polypeptide Sequence | NO: |
| CTADNGFGPQRSEVVSLFVTVPVSRPILTLRVPRAQAV | 95 |
| DNGFGPQRSEVVSLFVTVPVSRPILTLRVPRAQAV | 96 |
| CTADNGFGPQRSEVVSLFVTVPVSRPILTLRVPRA | 97 |
| CTADNGFGPQRSEVVSLFVTVPVSRPILTLRVPRAQE | 98 |
| YTADNGFGPQRSEVVSLFFTVPVSRPILTLRVPRAQAV | 99 |
| WTADNGFGPQRSEVVSLFVTVPVSRPILTLRVPRNQAV | 100 |
| CTADNGFGPQRSNVVSLFVTVPVSRPILTLRVPRAQAV | 101 |
| TPDNFFGPQRSEVVSLFVTVPVSRPILTLRVPRAQA | 102 |
| CTADNGFGPQRSEVVSLFVTVPVSRPILTLRVPRNQAV | 103 |
| CTADNFFGPQRSEVVSLFVTVPVSRPILTLRVPRAQA | 104 |

TABLE 8
Examples of variant polypeptides
of SEQ ID NO: 8.

| Variant Polypeptide Sequence | SEQ ID NO: |
|---|---|
| HSDTISLSVIVPVSRPILTFRAPRAQA | 105 |
| AQHSDTISLSVIVPVSRPILTFRAPRAQ | 106 |
| AQHSDTISLSVNVPVSRPILTFRAPRE | 107 |
| AQHSDTISLSVPVPVSRPILTFRAPRAQA | 108 |
| ADHSDTISLSVNVPVSRPILTFRAPRAQA | 109 |
| AFHSDTISLSVLVPVSRPILTFRAPRAQA | 110 |
| AQHSDTISLSVIVPVSRPWLTFRAPR | 111 |
| AQHSDTISLSVIVPVSRPILTFRAPRAQA | 112 |
| AQHSDTISLSVIVPVSRPIFFFRAPRAQA | 113 |
| AQHSDTISLSVIWVPVSRPILTFRAPRAQE | 114 |

TABLE 9
Examples of variant polypeptides
of SEQ ID NO: 9.

| Variant Polypeptide Sequence | SEQ ID NO: |
|---|---|
| PDVTATPARLLFFAPTRRAAPLEEM | 115 |
| GAEVPDVTATPARLLFFAPTRRAAPLE | 116 |
| GAEVPDVTATPARLLFFAPTRRAAPLNEM | 117 |
| GAEVPDNTATPARLLFFPDTRRAAPLEEM | 118 |
| GAEVPVTATPARLLFMAPTRLANPLDEM | 119 |
| GATPARVTATPARLLFFAPTRRAAPLEEM | 120 |
| GAEVPDVTATPARLLFFAPTWRPPLEE | 121 |
| GAEVPDNTATPARLLFFAPTRRAAPLEEM | 122 |
| GAEVPDVTATPARLLFFAPTRRAAPLA | 123 |
| GAEVPDVTATPARLLFFAPTIIAAPLEEM | 124 |

TABLE 10
Examples of variant polypeptides
of SEQ ID NO: 10.

| | SEQ |
|---|---|
| | ID |
| Variant Polypeptide Sequence | NO: |
| RLNAALREKTSLSANNATLEKQLIELTRTNE | 125 |
| RLNAALREKTSLSANNATLNKQLIELTRTNELLKSK | 126 |
| RLNPPLREKTSLSANNATLEKQLIELTRTNELLKSK | 127 |
| RLNPPLNWKTSLSANNWTLEhQLIELTRTNELLKSK | 128 |
| LREKTSLSANNATLEKQLIELTRTNELLKSK | 129 |
| RLNAALREKTSLSANNATLEKQLIELTRTNELLKSK | 130 |
| RLNAALREKPPLSANNATLEKQLIELTRTNELLKSK | 131 |
| RLNAALFVKTSLSANNATLEKQLIELTRTNELLKSK | 132 |
| RLNAALREKTSLSANNATLEKQQKNLTRTNELLKSK | 133 |
| NNALREKTSLSANNATLEKQLIYLTRTNELLKSK | 134 |

TABLE 11
Examples of variant polypeptides
of SEQ ID NO: 11.

| | SEQ |
|---|---|
| | ID |
| Variant Polypeptide Sequence | NO: |
| NGNQKNLRILSLELMKLRNKRETKMRGMMAKQEGME | 135 |
| SENGNQKNLRILSLNNMKLRNKRETKMRGMMAKQEGME | 136 |
| SENGNQKNLRILSLELMKLRNKRETKMRGPPAKQEGME | 137 |
| SENGNQKNLRILSLELHKLRNKQETKMRGMMAKQEGME | 138 |
| SENGNQKNLRILSLELMKLRNKRETKMRGMMANQEGME | 139 |
| SENGNQKNLYILSLELPFLRNKRETKMRGMMAKQEGM | 140 |
| SENGNQKNLRILSLEKQKLRNKRETKYRGMMAKQEGME | 141 |
| SKNGNQKNLRILSLELMKLRNKRETKMRGMMAKQEGME | 142 |
| SENGNQKNLRILSLELMKLINKRETKMRGMMAKQEGME | 143 |
| SENGNQKNLRILSLNLMKLRNKRETKMRGMMAKQEGME | 144 |

TABLE 12
Examples of variant polypeptides
of SEQ ID NO: 12.

| | SEQ |
|---|---|
| | ID |
| Variant Polypeptide Sequence | NO: |
| AHLQATLLLQEKYDSMVQSLEDVTAQFESYKA | 145 |
| AHTQATLLLQEKYDSMVQSLEDVTAQFES | 146 |
| ATLLLQEKYDSMVQSLEDVTAQFESYKA | 147 |
| AHTQATPPLQNKYDSMVQSLEDVTAQFESYKA | 148 |
| AHTQATLLLQYKYDSMVQSLWDVTAQFfSYKA | 149 |
| AHTQATLLLQEKYYSMIVQSLVDVTAQFESYNA | 150 |
| AHTQATLLLQYKYPSMVQSLEDVTAQFESYKA | 151 |
| AHTQKNLLLQNKYDSMVQSLPDVTAQFESYKA | 152 |
| AHTQATLLLQYKYDSMVQSLNFVTAQFFSYKA | 153 |
| AHTQATLLLQFFYDSMVQSLEDVTAQFESYKA | 154 |

TABLE 13
Examples of variant polypeptides
of SEQ ID NO: 13.

| | SEQ |
|---|---|
| | ID |
| Variant Polypeptide Sequence | NO: |
| YTWKALGQAANESHNGSILPISWRWGESDMTFICVAR | 155 |
| DVIYTWKALGQAANESHNGSILPISWRWGESDMTFI | 156 |
| DVIYTWKALGQAANESHNGSILPISWRWGESDMTFA | 157 |
| DVIYTWKNLGQAANESHNGSILPISWRWGESDMTFI | 158 |
| CVAR | |
| DVIYTWKALGQYYNESHNGSILPISWRWGESDMTFI | 159 |
| CVAR | |
| DVIYTWYYLGQWYNESHNGSILPISWNWGESDMTFI | 160 |
| CVAR | |
| DVIYTWQYLGQAANESHNGSILPISWRWGESDMTFI | 161 |
| CVAR | |
| DVNYTWKALGQVINESHNGSILPISWRWGESDMTFI | 162 |
| DVIYYWQENLGQAANESHNGSILPISWRWGESDMTF | 163 |
| ICVAR | |
| DVIYTWKALGQAANESHNGSVVPISWRWGESDMTFI | 164 |
| CVAR | |

TABLE 14
Examples of variant polypeptides
of SEQ ID NO: 14.

| | SEQ |
|---|---|
| | ID |
| Variant Polypeptide Sequence | NO: |
| LGLFLWFLKRERQEENNPKGRSSK | 165 |
| LWFLKRERQEENNPKGRSSKYGLL | 166 |
| LGLFLWFLKRERQEENNPKGRSSKYGLL | 167 |
| LGLFLWFLKRERQVDSIVWGRSSKYGLL | 168 |
| LGLFLWFLKRERQPPNNPKGRSSKYGLL | 169 |
| LGLFLWFLKRERQYYNNPKGRSSKYGLL | 170 |
| LGLFLWFLPRERQEENNPNGRSSKYGLL | 171 |
| LGLFLWFLKRERQNINNPKGRSSKYNLL | 172 |
| LGLFLWFLKRERQEENNPKGRSSKYGLA | 173 |
| LGLFLWFLKRYYQEKNNPKGRSSKYGLL | 174 |

TABLE 15
Examples of variant polypeptides
of SEQ ID NO: 15.

| | SEQ |
|---|---|
| | ID |
| Variant Polypeptide Sequence | NO: |
| NSPDRVKRPMNAFMVWSRGQRRKMAQENPKMHNSEISK | 175 |
| NSPDRVKRPMNAFMVWSRGQRRKMA | 176 |
| PDRVKRPMNAFMVWSRGQRRKMAQENPKMHNSEISK | 177 |
| NSPDRVKRPMNAFMVWSRGQPPKMAQENPKMHNSEISA | 178 |
| SPNRVKRPMNAFMVWSRGQRVKMAQENPKMHNSEISK | 179 |
| NSPDPVKRPMNFFMVWSLGQRRKMAQENPKMHNSEISK | 180 |
| NSPDRVKRPMNAFMVWSRGQRRKMAQNNPAMHNSEISK | 181 |
| NSPDRVKRPMNAFMVWSWGQRRKMAQYNPKMHNSEISE | 182 |
| NSPDRVYRPMNAFMVWSRGQRRKMAQENPKMHNSEISK | 183 |
| NSPDRVKRPMNAFMYFSRGQRRKMAQENPKMHNSE | 184 |

TABLE 16
Examples of variant polypeptides
of SEQ ID NO: 16.

| | SEQ |
|---|---|
| | ID |
| Variant Polypeptide Sequence | NO: |
| TEKRPFIDEAKRLRALHMKEHPDYKYRPRRK | 185 |
| EWKLLSETEKRPFIDEAKRLRALHMKEHPDYKYRP | 186 |
| EWKLLSYTEKRPFIYEANRLRALHMKEHPDYKYRPRRK | 187 |
| EWKLLSETEKRPFIDEAKRLYPLHMKNHPDYKYRPRRK | 188 |
| EWKLLSWTQYRPFIDEAKRLRALHMKEHPDYKYRPRRK | 189 |
| EWKLLYWTNKRPFIDEAKRLRALHMKEHPDYKYRPRRA | 190 |
| EWKLLSETEKRPFIDVSALQRALHMKEHPDYKYRPRRK | 191 |
| LSETEKRPFIDEAKRLRALHMKEHPDYKYRPRRK | 192 |
| EWKLLSETEKRPFIDMAKRLNALHMKEHPDYYYRPRRA | 193 |
| EWKLLSETYKRPFIMEAKRLRNLHMKEHPDYKY | 194 |

TABLE 17
Examples of variant polypeptides
of SEQ ID NO: 17.

| | SEQ |
|---|---|
| | ID |
| Variant Polypeptide Sequence | NO: |
| PNPADGTPKVYYPSGQPASAAGAPAGQ | 195 |
| AAAPNPADGTPKVLLLSGQPASAAGA | 196 |
| AAAPNPADGTPKVLLLSGQPASYYGAPAGQ | 197 |
| AAAPNPADGTPMVLLLSGQPASNNGAPAGQ | 198 |
| APNPATGTPKVLLLSGQPASAAGAPAGQ | 199 |
| AAPNPLYGTPKVLLLSGQPASAAGAPAGQ | 200 |
| AAAPNPADGTPKVLYLLSGQPASAAGAPAGQ | 201 |
| AAAPNPADGTPAVLLLSGQPASNNGAPAGQ | 202 |
| AAAPNPADGTPKVLLYLSGQPASAAGAPAGE | 203 |
| AAAPNPNDGTPKVLLLSGQPASNYGAPAGQ | 204 |

TABLE 18
Examples of variant polypeptides
of SEQ ID NO: 18.

| | SEQ |
|---|---|
| | ID |
| Variant Polypeptide Sequence | NO: |
| SYSDILLGILDNLDPVMFFKCPSPEPASLEELPEVYP | 205 |
| EGP | |
| SDILLGILDNLDPVMFFKCPSPEPASLEELPEVYP | 206 |
| SDILLGILDNLDPVMFFQCPSPNPASLYYLPEVYPE | 207 |
| SDILLGILDNLDPVMFFKCPSPdPASLVILPEVYPEGE | 208 |
| SDILLGILDNLDPVMFFNQPSPEPASLEELPEVYPEGP | 209 |
| SDILLGILDNLDPVMFFKCPSPEPASLEELPEVYQELP | 210 |
| SDILLGILDNLDPVMFFQNPSPEPASLEELPEVYPEGP | 211 |
| SDILLGILDNLDPVMFFKNPSPAPASLEELPEVYPEGP | 212 |
| SDILLGILDNLDPVMFFFVPSPTPASLEELPEVYPEGE | 213 |
| SDILLGILDNELIRMFFKCPSPEPASLEELPEVYPEGP | 214 |

TABLE 19
Examples of variant polypeptides
of SEQ ID NO: 19.

| | SEQ |
|---|---|
| | ID |
| Variant Polypeptide Sequence | NO: |
| PETVFKRPRECDSPSYQKRQRMALLARK | 215 |
| AVDPETVFKRPRECDSPSYQKRQRMALL | 216 |
| AVDPETVFKRPRYMDSPSYQKRQRMALLA | 217 |
| VDPATVFKRPynwDSPSYQKRQRMALLARK | 218 |
| AVDPETVFKRPRRKDSPSYQKRQRMALLARK | 219 |
| AVDPEYTVFKRPRVCDSPSYQKRQRMALLARK | 220 |
| AVDPVTVFRKPRECDSPSYQKRQRMALLARK | 221 |
| AVDPETVFKRPRYIDSPSYQKRQRMALLARK | 222 |
| AVDPPTVFFRPREYYSPSYQKRQRMALLARK | 223 |
| AVDPQTVFFRPREWDSPSYQKRQRMALLARK | 224 |

TABLE 20
Examples of variant polypeptides of SEQ ID NO: 20.

| Variant Polypeptide Sequence | SEQ ID NO: |
|---|---|
| VQGPTAVRKRFFESIIKEAARCMRRDFVKHL | 225 |
| LEGVQGPTAVRKRFFESIIKEAARCMRRD | 226 |
| LEGVQYPTAVRKRFFESIIKENFRCMRRDFVKHL | 227 |
| LEGVQGPTAVRKRFFESILTNRCMRRDFVKHL | 228 |
| LEGVQGPTAVRKRFFESIIKEPVRCMRRDFVKHL | 229 |
| LLGVQNPTAVRKRFFESIIKRKARCMRRDFVKHL | 230 |
| LEGVQGPTAVRKRFFESIIKEFFRCMRRDFVKHL | 231 |
| LEGWQGPTAVRKRFFESIINYPARCMRRDFVKHL | 232 |
| LEGVQGPTAVRKRFFESIIKEAARNMRRDFVKHL | 233 |
| LEGVQGPTAVRKRFFESIIKEAARQMRRDFVKH | 234 |

TABLE 21
Examples of variant polypeptides of SEQ ID NO: 21.

| Variant Polypeptide Sequence | SEQ ID NO: |
|---|---|
| TFPDLESEFQAALSRKVAELVHFLL | 235 |
| FPDLESEFQAALSRKVAELVHFLLLK | 236 |
| TFPDLESEFQAALSRKVAELVHFLLLK | 237 |
| TFPDLESEFQNYLSRKVAELVHFLLLK | 238 |
| TFPDLESEFQYYLSRKVANLVHFLLLK | 239 |
| TFPDLESEFQAPLSRKVAELVHFLLL | 240 |
| TFPDLESEFQAAVRRKVAELVHFLfLE | 241 |
| TFPDLESEFQSSLSRKVAELVHFLLLK | 242 |
| TFPDLESEFQAALSYYVAELVHFLLL | 243 |
| TFPDLESEFQANLSRNVAELVHFLLLK | 244 |

TABLE 22
Examples of variant polypeptides of SEQ ID NO: 22.

| Variant Polypeptide Sequence | SEQ ID NO: |
|---|---|
| SLFREALSNKVDELAHFLLRKYRAKEL | 245 |
| PDAESLFREALSNKVDELAHFLLRKYRA | 246 |
| PDAESYFREALSNKVDSLAHFLLRKYRAKEL | 247 |
| PDAESLFREALSNQVDELAHFLLRSYRAKEL | 248 |
| PDAESLMREALSNKVDELAHFQLRKYRAKEL | 249 |
| PDAESLFRQALSNKVDELAHIQLRKYRAKEL | 250 |
| PDAESLFREALSNKVDELAHFLLRKYRAK | 251 |
| VDAESLFREALSNKVDELAHFLLRKYRAKEL | 252 |
| PDAESLFRENLSNKVDELAHFLLRKYRAKE | 253 |
| PDAESLFREALSNKVDELAHFLLNKYRAKEL | 254 |

TABLE 23
Examples of variant polypeptides of SEQ ID NO: 23.

| Variant Polypeptide Sequence | SEQ ID NO: |
|---|---|
| PDLESEFQAALSRKVAKLVHFLLLK | 255 |
| TFPDLESEFQAALSRKVAKLVHFL | 256 |
| TFPDLESEFQNYLSRKVAKLVHFLLLK | 257 |
| TFPDLESEFQANLSRKVAKLVHFLLLE | 258 |
| TFPDLESEFQAALSRKVAKLVHFLLLK | 259 |
| TFPDLESEFQAALSRKVAKLVHFLYLL | 260 |
| FPDLNSEFQAALSRNVAKLVHFLLLK | 261 |
| TFPDLESEFQAALSRKVWKLVHFLLA | 262 |
| SFPDLESEFQAALSRKVNKLVHFLYLK | 263 |
| TFPDLESEFQAALSRKVYKLVHFLLLK | 264 |

TABLE 24
Examples of variant polypeptides of SEQ ID NO: 24.

| | SEQ |
|---|---|
| Variant Polypeptide Sequence | ID NO: |
| YLEYRQVPGSDPACYEFLWGPRALIETSYVKVLHHM | 265 |
| FVQENYLEYRQHfGSDPACYEFLWGPRALIETSYVKVLHHM | 266 |
| FVQENYLEMWQVPGSDPACYEFLWGPRALIETSYVKVLHHM | 267 |
| FVQENYLEYRQAAISDPACYEFLWGPRALIETSYVKVLHHM | 268 |
| FVQENYLEYRQVPGSDPACYEFLWGPRALIETSYVHFLHHM | 269 |
| FVQENYLEYRQVPGSDPACYEFLWGPRALIETSYVKVLHHM | 270 |
| FVQENYLEYRQVPGSDPAWYEFLWGPRALIETSYVKVLHHM | 271 |
| FVQENYLEYRQVPGSDPAYYEFLWGPRALIETSYVKVLHHM | 272 |
| FVQENYLEYRQVPGSDPACYEFFWGQRALIETSYVKVLHHM | 273 |
| FVQENYLEYRQVPGSDPACYIFMWGPRALIETSYVKVLHHE | 274 |

TABLE 25
Examples of variant polypeptides of SEQ ID NO: 25.

| Variant Polypeptide Sequence | SEQ ID NO: |
|---|---|
| PLQRPVSSFFSYTLASLLQSSHESPQS | 275 |
| LQRPVSSFFSYTLASLLQSSHESPQS | 276 |
| QRPVSSFFSYTLASLLQSSHESPQS | 277 |
| SPLQRPVSSFFSYTLASLLQSSHESPQ | 278 |
| SPLQRPVSTFFSYTLASLLQSSHESPQQ | 279 |
| SPLQRPVSSFFSYTLASLLQSSHESPQN | 280 |
| SPLQRPVWSFFSYTLASLLQSSHESPQA | 281 |
| QPLQRPVSSFFSYTLASLIQSSHESPQS | 282 |
| NPLQRPVSSFFSYTLASLLQSSHESPQS | 283 |
| APLQRPVSSFFSYTLASLLQSSHESPQS | 284 |

TABLE 26
Examples of variant polypeptides of SEQ ID NO: 26.

| Variant Polypeptide Sequence | SEQ ID NO: |
|---|---|
| SSTSSSLSKSSPESPLQSPVISFS | 285 |
| SSLSKSSPESPLQSPVISFSA | 286 |
| SSTSASLSKSSPESPLQSPVISFSN | 287 |
| SSTSSSLSKNSPESPLQSPVIS | 288 |
| SSTSSSLSKSNPESPLQSPVISFSS | 289 |
| SSTSYYLSKSSIESPLQSPVISFSS | 290 |
| SSTSSSLSKSSVESPLQSPVISFSS | 291 |
| SSTSWSLSKTSPQSPLQSPVISFSE | 292 |
| SSTSMSLSKSSPNSPLQSPVISFSA | 293 |
| SSTSSSLSKSSPENPLQSPVISFSS | 294 |

TABLE 27
Examples of variant polypeptides of SEQ ID NO: 27.

| Variant Polypeptide Sequence | SEQ ID NO: |
|---|---|
| PRYEFLWGPRAHSEVIKRKVVEFLAMLKNTVPI | 295 |
| NSSPPRYQFLWGVRAHSEVIKRKVVEFLAMLKNTVPI | 296 |
| NSSPPRYEFLWGPRAHSEVIKRKVVEFLAML | 297 |
| NSTPPRYEFLWNPRAHSEVIIRKVVEFLAMLKNTVPI | 298 |
| SPPRYEFLWGPNAHSEVIKRKVVEFLAMLKNTVPI | 299 |
| NSSPPRYNFLWGPRAHSIVIARKVVEFLAMLRNTVPI | 300 |
| NSSPPRYEFLWGPRNHSEVIKRKVVEFLVMLKNTVPI | 301 |
| RYEFLWGPRAHSEVIKRKVVEFLAMLKNTVPI | 302 |
| NSSPPRYEFLWGPRAHSEVIKRKVVEFLAMLKNTVPI | 303 |
| SSPPRYEFLWGPRAHSEVIKRKVVEFLAMLKNTL | 304 |

TABLE 28
Examples of variant polypeptides of SEQ ID NO: 28.

| Variant Polypeptide Sequence | SEQ ID NO: |
|---|---|
| ARGPESRLLEFYLAMPFATPMEAELARRSLA | 305 |
| GPESRLLEFYLIMPFATPMEAELARRSLAQDAPPL | 306 |
| ARGPESRLLKFYLVMPFATPMEAELARRSLAQDAPPL | 307 |
| ARGPESRLLKFYLTMPFATPMEAELARDSLAQDAPPL | 308 |
| ARGPESRLLEFYLAMPFATPMEAELARRSLAQDA | 309 |
| ARGPESRLLEFYLAMPFATPDTRPLARRSLAQDAPPL | 310 |
| ARGPESRLLKFYLAMPFATPMEAELARRSLAQDAPPL | 311 |
| ARGPNSRLLIFYLYCPFATPMRARLSRRTLAQDAPPL | 312 |
| ARGPESRLLEFYLAMPFATPMEAELQNQSLAQDAPPL | 313 |
| RGPESRLLEFYLAMPFATPMEAELARRSLAQDAPP | 314 |

TABLE 29
Examples of variant polypeptides of SEQ ID NO: 29.

| Variant Polypeptide Sequence | SEQ ID NO: |
|---|---|
| VPGVLLKEFTVSGNILTIRLTAADHRQLQLS | 315 |
| PGVLLKEFTVSGNILTIRLTAADHRQL | 316 |
| VPGVLLKEFTVSGNILTIRALTWYDHRQL | 317 |
| VPGVLLKEFTVSGNILTRRLTAADHRQL | 318 |
| PGVLLKEFTVSGNILTIRLTAADHRQLE | 319 |
| VPGVLLKEFTVSGNILTIRLTKMDHRQL | 320 |
| VPGVLLKEFTVSGNILTWRLTAADHRQL | 321 |
| VPGVLLKEFTVSGNILTWQLTMADHRQL | 322 |
| VPGVLLKEFTVSGNILTIRLTAADHRQLDA | 323 |
| VPGVLLKEFTVSGNILTIRLTSTDHRQL | 324 |

TABLE 30
Examples of variant polypeptides of SEQ ID NO: 30.

| Variant Polypeptide Sequence | SEQ ID NO: |
|---|---|
| RSASETSEKRPFMCAYPGCNKRYFKLSE | 325 |
| SETSEKRPFMYAYPGCNKRYFLSHLQMH | 326 |
| RSASETSNKRPFMCAYPGCNKRYFKLSHLQMH | 327 |
| RSASETSYQRPFMCAYPGCNKRYFKLSHLQMH | 328 |
| RSASETSFQRPFMCAYPGCNKRYFKLSHLQMH | 329 |
| RSASETLEKRPFMCAYPGCNKRYFLLSHLQMH | 330 |
| RSASWTSEKRPFMCAYPGCNKRYFKLNHSFKH | 331 |
| RSASSTSEKRPFfCAYPGCNKRYFKLSHLQMH | 332 |
| RSASETYSVRPFMCAYPGCNKRYFKLSHLQMH | 333 |
| RSASETSIKRPFYCAYPGCNKRYFKLSHLQMH | 334 |

TABLE 31
Examples of variant polypeptides of SEQ ID NO: 31.

| Variant Polypeptide Sequence | SEQ ID NO: |
|---|---|
| VHCCLYFIPATGHSLRPLDIEFMKRLSKVVNIVP | 335 |
| DTRVHCCLYFIPDTRGSLRPLDIEFMKRLSKVVNIVP | 336 |
| DTRVHCCLYFIPATGHSLRPLDIEFMKRLSKVVNIV | 337 |
| DTRVHCCLYFIPATGHSLRPLDIEFMKRLSKV | 338 |
| DTRVHCCLYFIPATGHSLIPLDILFMKRLSNVVNIVP | 339 |
| DTRVHKWLYFIPATGHSLRPLDIEFMKRLSKVVNIVP | 340 |
| DTRVHCLLYFIPATGHSLRPLDIEFMKRLSKVVNIVP | 341 |
| DTRVHCCLYFIPVTRHSLRPLDINFMKRLSKVVNIVP | 342 |
| DTRVHCCLYFIPATGHSLRPLDIHFMKRLSKVMNIVP | 343 |
| DTRVHCCLYFIPATSASLNPLDIEFMKRLSTVVNIVP | 344 |

TABLE 32
Examples of variant polypeptides of SEQ ID NO: 32.

| Variant Polypeptide Sequence | SEQ ID NO: |
|---|---|
| RLVNEKFREMIPFAVVGSDHEYQVNGKRIL | 345 |
| LVNEKFREMYPFNVVGSDHEYQVNGK | 346 |
| LVNEKFREMIPFAVVGSDHWYQVNGKRIL | 347 |
| LVNEKFRLAIPFAVVGSDHEYQVNGKRIL | 348 |
| LVNEKFREMIPFQVVGSDHNYQVNGKRIL | 349 |
| LVNEKFREMIPFAVVGSTHYYQVNGKRIL | 350 |
| RLVNPKFREMIPFAVVGSDHNYQVNGKRIL | 351 |
| LVNWKFRFMIPFAVVGSDHEYQVNGKQIL | 352 |
| LVNEKFREMIPFAVVGSLHIYQVNMKRIL | 353 |
| LVNEKFREMIPFAVVGSTHVYQVNGKRIL | 354 |

TABLE 33
Examples of variant polypeptides of SEQ ID NO: 33.

| Variant Peptide Sequence | SEQ ID NO: |
|---|---|
| FAYLRDLLIRTHMQNIKDITSSIHFEAYR | 355 |
| TTHCEFAYLRDLLIRTHMQNIKDITSSIHF | 356 |
| TTHWNFAYLRDLLIRTHMQNIKDITYTIHFEAYR | 357 |
| TTHCEFAYLRDLLIRTHMQNINDITLSIHFEAYE | 358 |
| TTHCEFAYLRDLLIRTHMQNIKDITYYIHFEAYA | 359 |
| TTHVWFAYLRDLLIRTHMQNIKDITSSIHFEAYR | 360 |
| TTHCEFAYLRLLLIRTHMQNIKDITSSIHFEAYR | 361 |
| TTHPEFAYLRDLLIRTHMQNIKDITSSIHFEAYR | 362 |
| FAYLRDLLIRTHMQNIKDITSSIHFEAYR | 363 |
| THCEFAYLRDLLIVVQHMQNIKDITSSIHFEAYR | 364 |

A polypeptide provided herein (e.g., an isolated polypeptide that comprises, consists essentially of, or consists of the amino acid sequence set forth in any one of SEQ ID NOs: 1-33 or a variant polypeptide provided herein) can have the ability to be naturally processed and presented by different MHC molecules. For example, after contacting cells (e.g., T cells) with one or more polypeptides provided herein (e.g., a polypeptide set forth in FIG. 1B or any one of Tables 1-33), the T cells can be activated to generate antigen-specific T cells having a desired antigen specificity.

Any appropriate method can be used to obtain a polypeptide provided herein (e.g., an isolated polypeptide that comprises, consists essentially of, or consists of the amino acid sequence set forth in any one of SEQ ID NOs: 1-33 or an isolated variant polypeptide provided herein). In some cases, a polypeptide provided herein can be obtained using polypeptide synthesizing methods. For example, a polynucleotide sequence encoding a polypeptide provided herein can be inserted into a plasmid or other vector that can then be delivered to hosts that can be induced to transcribe and translate the polynucleotide into the polypeptide. In some cases, a polynucleotide sequence for a larger polypeptide can be inserted into host cells that can produce the larger polypeptide and then process that polypeptide into a smaller polypeptide or a functional variant of interest.

This document also provides compositions containing one or more polypeptides provided herein. In some cases, a polypeptide that comprises, consists essentially of, or consists of the amino acid sequence set forth in any one of SEQ ID NOs: 1-33 (or a variant polypeptide provided herein) can be used individually to produce a composition. In some cases, a mixture of two or more polypeptides provided herein (e.g., two or more variant polypeptides and/or polypeptides that comprise, consist essentially of, or consist of the amino acid sequence set forth in any one of SEQ ID NOs: 1-33) can be used to produce a composition. Any appropriate combination of the polypeptides listed in FIG. 1B and/or Tables 1-33 can be used to produce a composition. For example, the combination can include at least 2, 3, 4, 5, 6, 7, 8, 9, 10, 11, 12, 13, 14, 15, 16, 17, 18, or more polypeptides selected from FIG. 1B and Tables 1-33. Examples of specific combinations of polypeptides that can be used to make a composition provided herein include, without limitation, those set forth in Table 34.

TABLE 34
Exemplary combinations of polypeptides.

| Composition | |
|---|---|
| Number | Combination of polypeptides |
| 1 | SEQ ID NOs: 1-3 |
| 2 | SEQ ID NOs: 1, 20, 23, 24, and 31 |
| 3 | SEQ ID NOs: 4, 7, 8, 13, and 14 |
| 4 | SEQ ID NOs: 10, 11, 12, 28, and 29 |
| 5 | SEQ ID NOs: 1-4, 10, 12, 13, 20, 28, and 29 |
| 6 | SEQ ID NOs: 2, 4, 12, 13, and 29 |
| 7 | SEQ ID NOs: 1, 10, and 20 |
| 8 | SEQ ID NOs: 2, 4, and 29 |
| 9 | SEQ ID NOs: 3, 12, and 13 |
| 10 | SEQ ID NOs: 11, 14, and 28 |
| 11 | SEQ ID NOs: 28 and 29 |
| 12 | SEQ ID NOs: 13 and 14 |
| 13 | SEQ ID NOs: 19 and 20 |
| 14 | SEQ ID NOs: 10-12 |
| 15 | SEQ ID NOs: 1-4, 10, 12, 13, 20, and 29 |

In some cases, a composition provided herein (e.g., a composition containing one or more polypeptides that comprise, consist essentially of, or consist of the amino acid sequence set forth in any one of SEQ ID NOs: 1-33 and/or variant polypeptides provided herein) also can include one or more polypeptides as described elsewhere (see, e.g., WO 2017/096247).

In some cases, a composition provided herein (e.g., a composition containing one or more polypeptides that comprise, consist essentially of, or consist of the amino acid sequence set forth in any one of SEQ ID NOs: 1-33 and/or variant polypeptides provided herein) can be designed to activate T cells in culture. For example, a composition provided herein can be used to activate T cells obtained from a mammal (e.g., a human) to generate antigen-specific T cells against cancer cells or precancerous cells expressing one or more of the polypeptides.

Any appropriate method can be used to formulate a composition provided herein (e.g., a composition containing one or more polypeptides that comprise, consist essentially of, or consist of the amino acid sequence set forth in any one of SEQ ID NOs: 1-33 and/or variant polypeptides provided herein). For example, one or more polypeptides provided herein can be combined with a pharmaceutically acceptable carrier and/or a pharmaceutical excipient. The term “pharmaceutically acceptable” refers to generally non-toxic, inert, and/or physiologically compatible compounds. A term “pharmaceutical excipient” includes materials such as carriers, pH-adjusting and buffering agents, tonicity adjusting agents, wetting agents, colorants, and preservatives.

This document also provides methods and materials for activating T cells. For example, one or more polypeptides provided herein (e.g., a composition that contains one or more polypeptides provided herein) can have the ability to activate T cells obtained from a mammal (e.g., a human) in culture. In some cases, one or more polypeptides provided herein (e.g., a composition that contains one or more polypeptides provided herein) can be contacted with T cells to generate antigen-specific T cells (e.g., antigen-specific CD4⁺ and/or antigen-specific CD8⁺ T_{EM} cells and/or antigen-specific CD4⁺ and/or antigen-specific CD8⁺ T_{CM} cells) having a desired antigen specificity. For example, one or more polypeptides provided herein can be contacted with naïve T cells to generate T_{EM} cells and/or T_{CM} cells that can target (e.g., target and destroy) cells (e.g., cancer cells or precancerous cells) expressing the one or more polypeptides. Activated T cells can be used in an immunotherapy (e.g., adoptive T-cell therapy), and can be administered to a mammal (e.g., a human) to induce an immune response against cancer cells or precancerous cells within the mammal.

Any appropriate type of cancer or precancerous condition can be treated using the methods and materials provided herein. In some cases, a cancer, or a precancerous condition, to be treated using the methods and materials provided herein can include one or more cancer cells or precancerous cells that express one or more cancer antigen polypeptides described herein. In some cases, a cancer can include one or more solid tumors. In some cases, a cancer can be a blood cancer. In some cases, a cancer can be a primary cancer. In some cases, a cancer can be a metastatic cancer. Examples of cancers and precancerous conditions that can be treated using the methods and materials provided herein include, without limitation, MM, MGUS, (e.g., smoldering MM), colorectal cancer, breast cancer, colon cancer, rectal cancer, prostate cancer, endometrial cancer, cervical cancer, gastric cancer, kidney cancer, pancreatic cancer, brain cancer, head and neck cancer, lung cancer, salivary gland cancer, ovarian cancer, fallopian tube cancer, uterus cancer, esophageal cancer, cholangiocarcinoma, glioblastoma, neuroblastoma, non-Hodgkin's lymphoma, and melanoma.

One or more polypeptides provided herein (e.g., a composition that contains one or more polypeptides provided herein) can be contacted with any appropriate cell population (e.g., a cell population containing naïve T cells) to activate T cells within that cell population. In some cases, a cell population to be contacted with one or more polypeptides provided herein can be obtained from a mammal (e.g., a human) to be treated with activated T cells generated as described herein. Examples of cell populations that can be contacted with one or more polypeptides provided herein to activate T cells within the cell population to make populations of antigen-specific T cells (e.g., antigen-specific CD4⁺ and/or antigen-specific CD8⁺ T_{EM} cells and/or antigen-specific CD4⁺ and/or antigen-specific CD8⁺ T_{CM} cells) include, without limitation, peripheral blood cells (e.g., peripheral blood mononuclear cells (PBMCs) such as unfractionated PBMCs), tumor samples that contain cells, lymph node samples that contain cells, spleen samples that contain cells, bone marrow samples that contain cells, cerebrospinal fluid samples that contain cells, pleural fluid samples that contain cells, peritoneal fluid samples that contain cells, and joint fluid samples that contain cells.

Any appropriate method can be used to contact a cell population (e.g., a cell population containing naïve T cells) with one or more polypeptides provided herein (e.g., a composition that contains one or more polypeptides provided herein) to activate T cells within that cell population. For example, one or more polypeptides provided herein can be cultured with a cell population (e.g., a cell population containing naïve T cells) to activate T cells within that cell population. In some cases, a population of cells can be cultured in a manner that promotes antigen presentation. In some cases, a population of cells can be cultured with a cell population (e.g., a cell population containing naïve T cells) to activate T cells within that cell population as described in Example 1. In some cases, a population of cells can be cultured in a manner that promotes antigen presentation. In some cases, a population of cells can be cultured with a cell population (e.g., a cell population containing naïve T cells) to activate T cells within that cell population as described elsewhere (see, e.g., WO 2017/034833).

A cell population (e.g., a cell population containing naïve T cells) can be contacted with any appropriate amount of one or more polypeptides provided herein (e.g., a composition that contains one or more polypeptides provided herein) to activate T cells within that cell population. In some cases, from about 5 μg/mL to about 100 μg/mL of total polypeptides provided herein can be contacted with a cell population (e.g., a cell population containing naïve T cells) to activate T cells within that cell population. For example, 5 μg/mL, 10 μg/mL, or 25 μg/mL of total polypeptides can be contacted with a cell population (e.g., a cell population containing naïve T cells) to activate T cells within that cell population.

Once a population of antigen-specific T cells (e.g., antigen-specific CD4⁺ and/or antigen-specific CD8⁺ T_{EM} cells and/or antigen-specific CD4⁺ and/or antigen-specific CD8⁺ T_{CM} cells) is obtained as described herein, the cells can be administered to a mammal for use in, for example, adoptive cellular therapies to treat cancer (e.g., MM) or a precancerous condition (e.g., MGUS). In some cases, a population of antigen-specific T cells (e.g., antigen-specific CD4⁺ and/or antigen-specific CD8⁺ T_{EM} cells and/or antigen-specific CD4⁺ and/or antigen-specific CD8⁺ T_{CM} cells) obtained as described herein can be administered to a mammal having cancer or a precancerous condition under conditions effective to reduce the severity of one or more symptoms of the cancer or precancerous condition and/or to reduce the number of cancer cells or precancerous cells present within the mammal. Treatment of individuals having cancer or a precancerous condition can include the administration of a therapeutically effective amount of antigen-specific T cells (e.g., antigen-specific CD4⁺ and/or antigen-specific CD8⁺ T_{EM} cells and/or antigen-specific CD4⁺ and/or antigen-specific CD8⁺ T_{CM} cells) obtained as described herein. The term “therapeutically effective amount” as used with treating cancer or a precancerous condition refers to that amount of the agent sufficient to reduce one or more symptoms of the cancer of precancerous condition and/or to reduce the number of cancer cells or precancerous cells within a mammal. In providing a mammal with a population of antigen-specific T cells obtained as described herein capable of inducing a therapeutic effect, the number of antigen-specific T cells will vary depending upon such factors as the subject's age, weight, height, sex, general medical condition, previous medical history, etc.

Any appropriate mammal can be treated with antigen-specific T cells (e.g., antigen-specific CD4⁺ and/or antigen-specific CD8⁺ T_{EM} cells and/or antigen-specific CD4⁺ and/or antigen-specific CD8⁺ T_{CM} cells) provided herein. For example, humans, non-human primates, horses, cattle, pigs, dogs, cats, mice, and rats can be treated with a population of antigen-specific T cells (e.g., antigen-specific CD4⁺ and/or antigen-specific CD8⁺ T_{EM} cells and/or antigen-specific CD4⁺ and/or antigen-specific CD8⁺ T_{CM} cells).

When antigen-specific T cells (e.g., antigen-specific CD4⁺ and/or antigen-specific CD8⁺ T_{EM} cells and/or antigen-specific CD4⁺ and/or antigen-specific CD8⁺ T_{CM} cells) provided herein are administered to a mammal (e.g., a human) as described herein, any appropriate number of antigen-specific T cells provided herein can be administered to the mammal. For example, from about 1×10³ to about 5×10¹¹ (e.g., from about 1×10⁴ to about 5×10¹¹, from about 1×10⁵ to about 5×10¹¹, from about 1×10⁶ to about 5×10¹¹, from about 1×10⁷ to about 5×10¹¹, from about 1×10⁸ to about 5×10¹¹, from about 1×10⁹ to about 5×10¹¹, from about 1×10¹⁰ to about 5×10¹¹, from about 1×10³ to about 1×10¹¹, from about 1×10³ to about 1×10¹⁰, from about 1×10³ to about 1×10⁹, from about 1×10⁶ to about 1×101⁰, from about 1×10⁷ to about 1×10¹⁰, from about 1×10⁸ to about 1×10¹⁰, or from about 1×10⁹ to about 1×10¹¹) T cells including antigen-specific T-cells can be administered to a mammal.

When antigen-specific T cells (e.g., antigen-specific CD4⁺ and/or antigen-specific CD8⁺ T_{EM} cells and/or antigen-specific CD4⁺ and/or antigen-specific CD8⁺ T_{CM} cells) provided herein are administered to a mammal (e.g., a human) as described herein, any appropriate route of administration can be used to administer the antigen-specific T cells provided herein to a mammal. For example, antigen-specific T cells can be administered intravenously, intraperitoneally, subcutaneously, intratumorally, intramuscularly, intrahepatically, or intranodally.

The invention will be further described in the following examples, which do not limit the scope of the invention described in the claims.

### EXAMPLES

### Example 1: Multipeptide Stimulated PBMCs Generate T_{EM} T_{EM} for Adoptive Cell Therapy in Multiple Myeloma

Multiple Myeloma (MM) patients suffer disease relapse due to the development of therapeutic resistance. Increasing evidence suggests that immunotherapeutic strategies can provide durable responses. This Example describes the design of polypeptides from antigens (Ags) that are over expressed in MM.

### Results

Synthetic polypeptides designed using NetMHCpan server: Polypeptides were designed from the following Ags: BCMA, MUC1, FcRH5, MCL1, RHAMM, SLAMF7, XBP(S)1, CT45, MAGEA3/6, NY-ESO-1, SEPT9 and WT1. NetMHCpan server employs artificial neural network to predict the binding affinity of polypeptides to MHC I or II. In FIG. 1A, the regions highlighted in bold represent MHC I hotspots, whereas those in italicized bold indicate high binding affinity to MHC II. The polypeptide length ranged between 17-41 mers and included high binding affinity for both MHC I and II. These polypeptides covered class I and II alleles from 90% of the US population encompassing Caucasians, African Americans, Hispanics, Asians, and American Indians. The list of polypeptides predicted to induce Ag-specific CD4⁺ and CD8⁺ T cell responses are as shown in FIG. 1B.

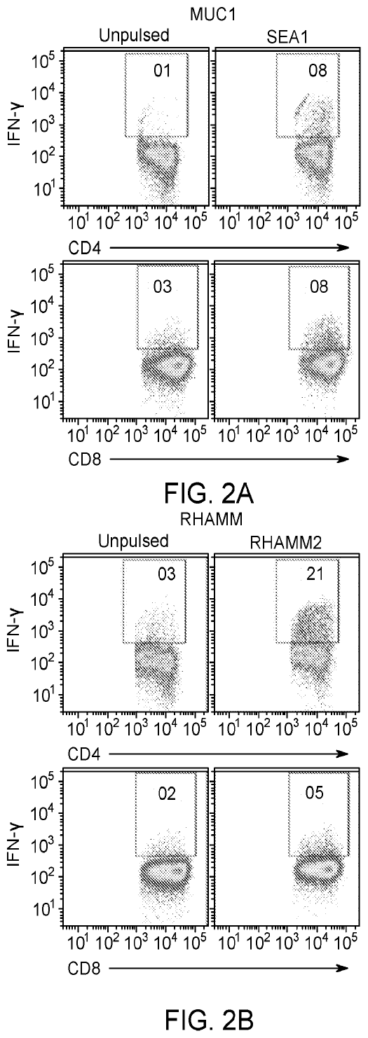

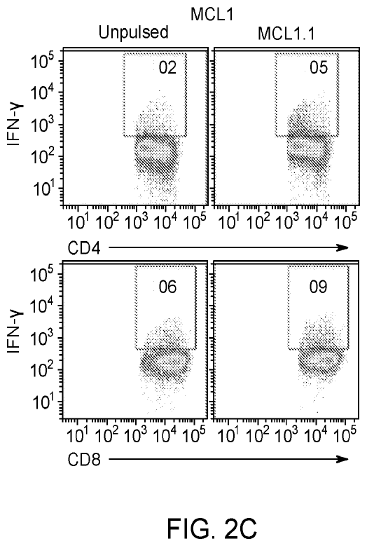

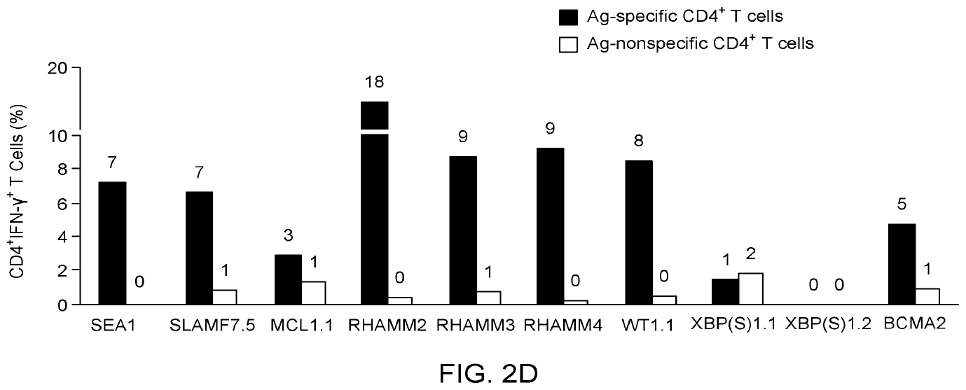

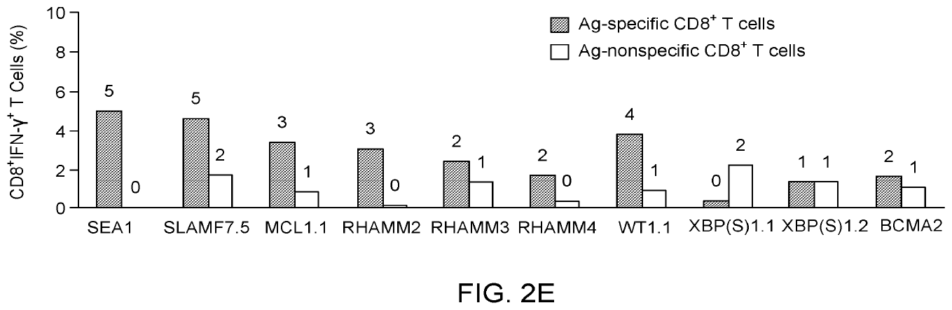

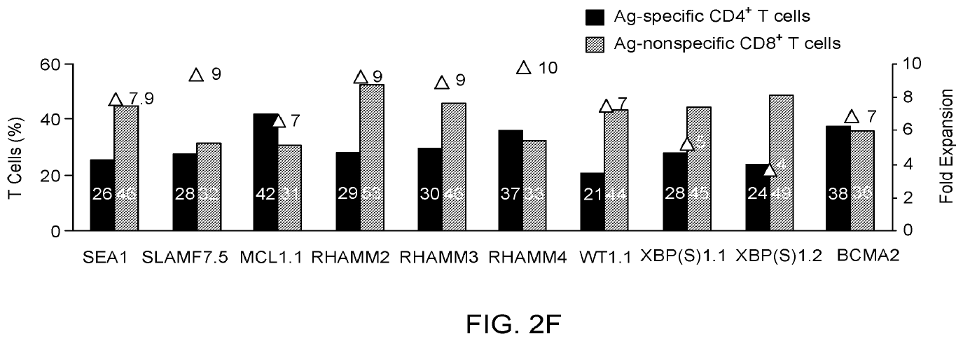

Single polypeptides induced activation of CD4⁺ and CD8⁺ in HD PBMCs: Unfractionated PBMCs from different HDs were stimulated with synthetic long polypeptides along with granulocyte-macrophage colony-stimulating factor (GM-CSF) and toll-like receptor (TLR) agonists 4 and 8 to activate innate immune cells. During the culture period that lasted for 19 days, T cell proliferation and survival were supported with interleukin-7 (IL-7), a T cell growth factor. In FIG. 2, the HD PBMCs were stimulated on day 0 with the polypeptides from the following Ags: MUC1 (SEA1), RHAMM (RHAMM2, 3, and 4), MCL1.1, SLAMF7.5, WT1.1, XBP(S)1.1, XBP(S)1.2 and BCMA2. The numbers for RHAMM2, 3, and 4, indicate that the polypeptides were designed from different regions of the same Ag. At the end of the culture period, T cells were harvested for secondary stimulation with PBMCs that were either unpulsed or pulsed with Ags similar or dissimilar from that used for primary stimulation to examine intracellular interferon-gamma (IFN-γ) expression in CD4⁺ and CD8⁺ T cells. Secondary stimulation of CD4⁺ T cells with unpulsed PBMCs resulted in IFN-γ expression that served as background (FIGS. 2A-2C, left panel). The re-stimulation of T cells with PBMCs pulsed with the specific polypeptide (SEA1, RHAMM2, or MCL1.1) used for primary stimulation led to robust IFN-γ expression in CD4⁺ T cells for MUC1 and RHAMM (FIGS. 2A-2C, top panel) and CD8⁺ T cells (FIGS. 2A-2C, bottom panel). In general, the response of CD4⁺ T cells was stronger than that of CD8⁺ T cells. The IFN-γ expression by CD4⁺ (FIG. 2D) and CD8⁺ (FIG. 2E) T cells was calculated by subtracting the background unpulsed value from that observed following restimulation with the specific polypeptide. The secondary exposure of T cells with an Ag different from that used for primary stimulation was considered as a negative control that is indicative of cross-reactivity. The stimulation of PBMCs with different polypeptides increased the total number of T cells (fold expansion) as well as enlarged both CD4⁺ and CD8⁺ T cell subsets (FIG. 2F).

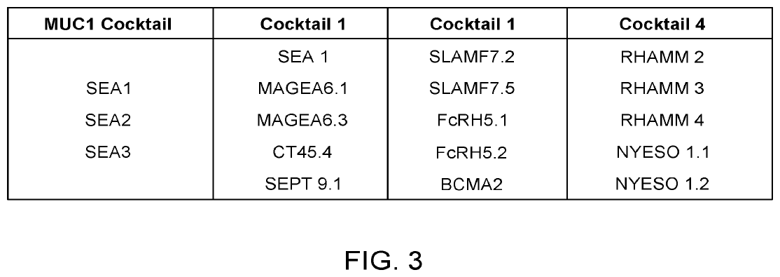

Amongst the Ags depicted here, MUC1, SLAMF7, RHAMM, WT1, and BCMA showed a robust Ag-specific T cell response. The polypeptides designed from MCL1 and XBP(S)1 gave a lower level of Ag-specific T cells. Overall, most of the novel polypeptides successfully induced Ag-specific CD4⁺ and CD8⁺ T cell responses. The efficacy of all polypeptides designed from different Ags was tested. The polypeptides that reproducibly activated naïve CD4⁺ and CD8⁺ T cells in an Ag-specific manner were selected to assemble polypeptide cocktails consisting of three or five polypeptides. The four polypeptide cocktails employed in the ensuing studies are depicted in FIG. 3. Immunization with multiple polypeptides can alleviate immune editing and dependency on a single Ag. Although the presence of multiple Ags in one mixture can lead to Ag competition, these studies showed that combinations can be devised that lead to strong activation of T cells to multiple polypeptides.

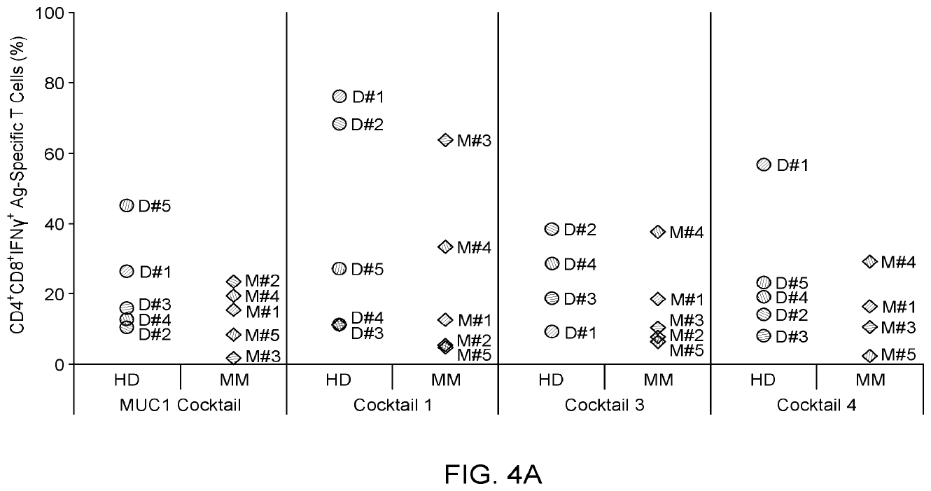

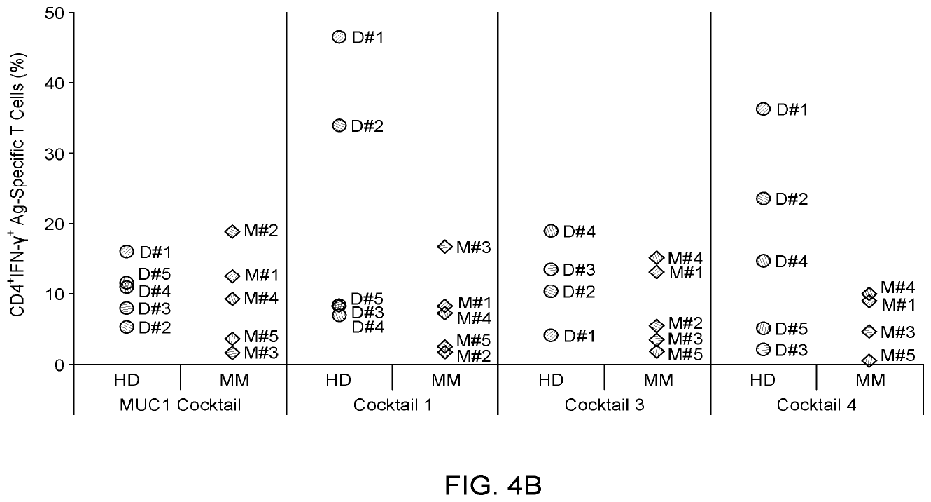

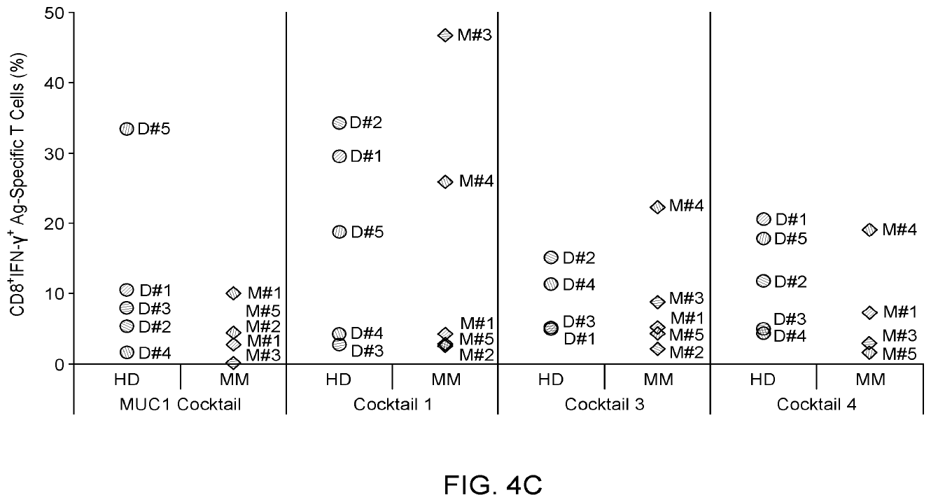

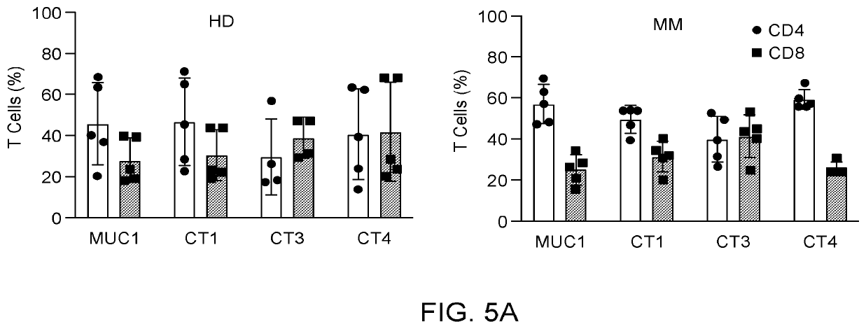

Peptide cocktails generate Ag-specific CD4⁺ and CD8⁺ T cells from PBMCs isolated from MM patients or HDs: To assess the ability of compiled polypeptide cocktails Mucin 1 Cocktail (MUC1 CT), Cocktail 1 (CT1), Cocktail 3 (CT3) and Cocktail 4 (CT4) to stimulate naïve T cells, PBMCs from HDs as well as MM patients that were at different disease stages were employed. PBMCs were exposed to different cocktails. The T cells harvested on day 19 were restimulated with a single polypeptide, which corresponded to each polypeptide that was present in the cocktail. Overall, as seen with HD PBMC, the polypeptide cocktails induced proliferation of Ag-specific CD4⁺ and CD8⁺ T cell responses following stimulation of PBMCs from MM patients, indicating the functional status of the immune system regardless of the presence of the disease (FIG. 4). The differing levels of responses to the various polypeptides may be due to different HLA types of the individual tested. No statistically significant differences were observed between the different cocktails or between the HDs and MM patients in each cocktail (Student's t-test p>0.1 in every comparison).

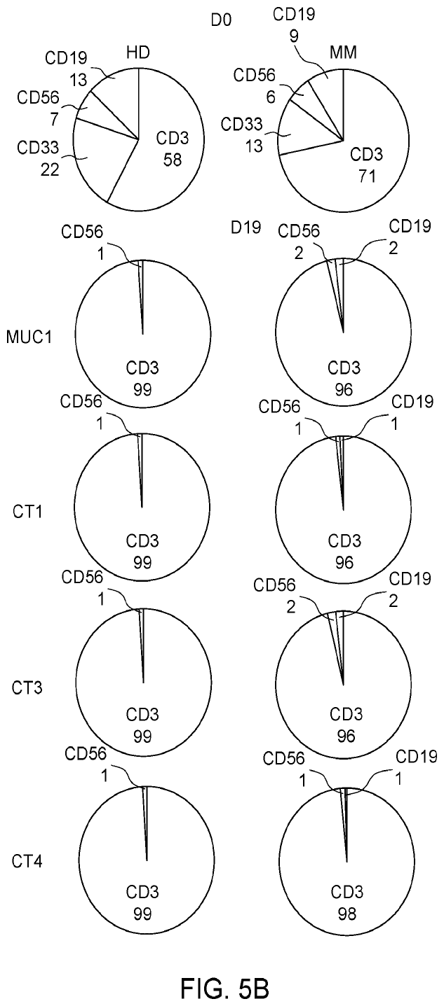

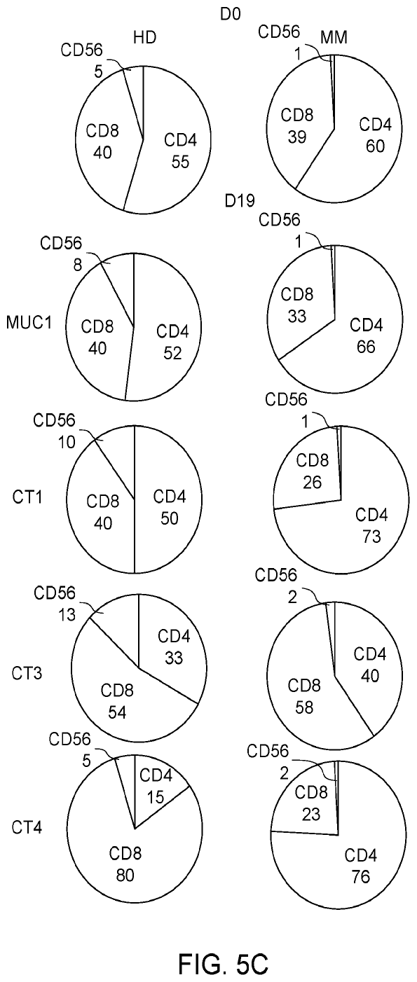

Subset evaluation indicated similar results for HD and MM patients. The stimulation of PBMCs with different polypeptides increased the total number of T cells (fold expansion) as well as enlarged both CD4⁺ and CD8⁺ T cell subsets (FIG. 5A). The day 0 PBMCs and cells harvested on day 19 were examined for the levels of different cell populations, such as CD3 (T cells), CD33 (myeloid cells), CD56 (Natural Killer Cells, NK cells), and CD19 (B cells). The 19 day culture resulted in a large expansion of CD3⁺ T cells, from about 50% at day 0 to greater than 90% on day 19 in all of the cocktails, whereas the CD33, CD56 and CD19 cell percentages decreased greatly (FIG. 5B). Analysis of the CD3⁺ T cells showed the majority were either CD4⁺ or CD8⁺, with the actual percentages varying depending upon the cocktail used for stimulation. There was a smaller percentage of CD3⁺CD56⁺ NKT cells, usually 10% or less. There were no statistically significant differences between the percentages of the different cell populations of MM patient and HD. The data shown are representative of the samples studied.

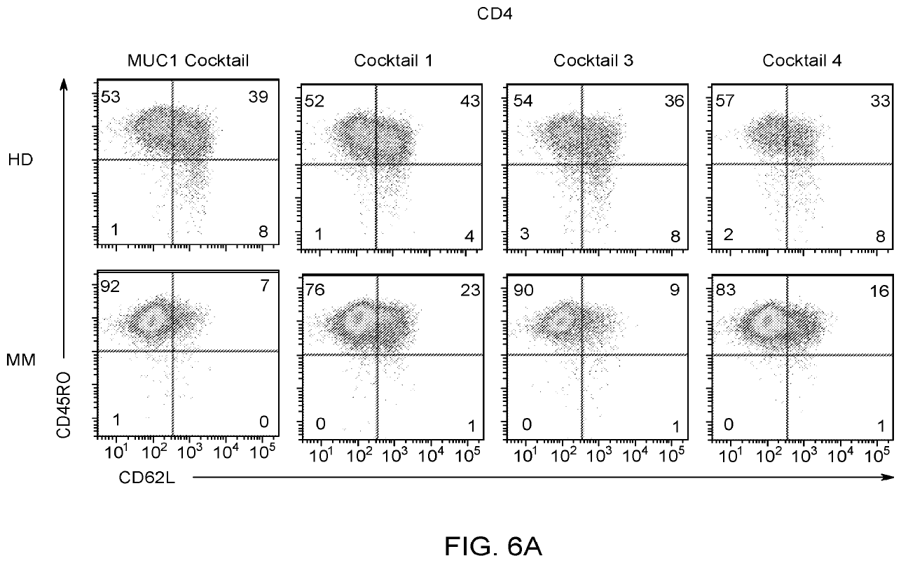

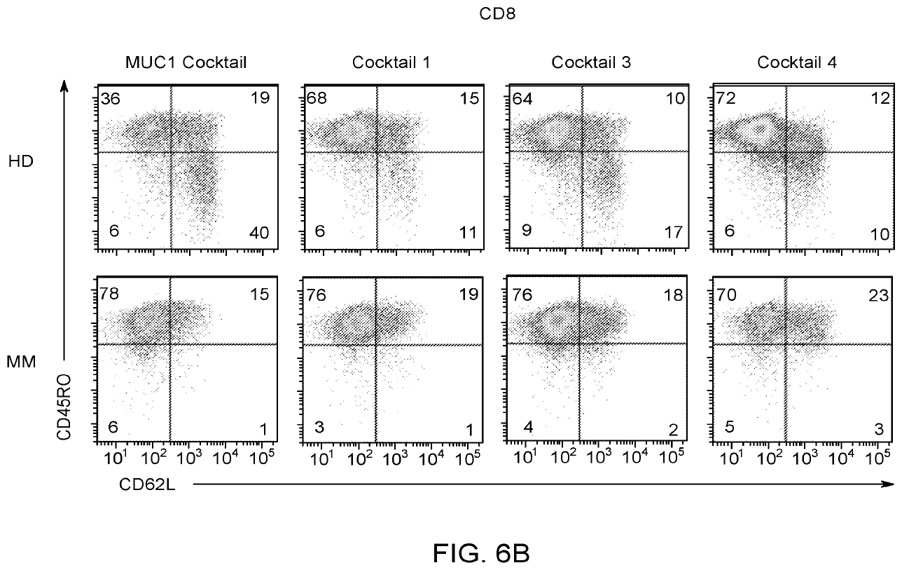

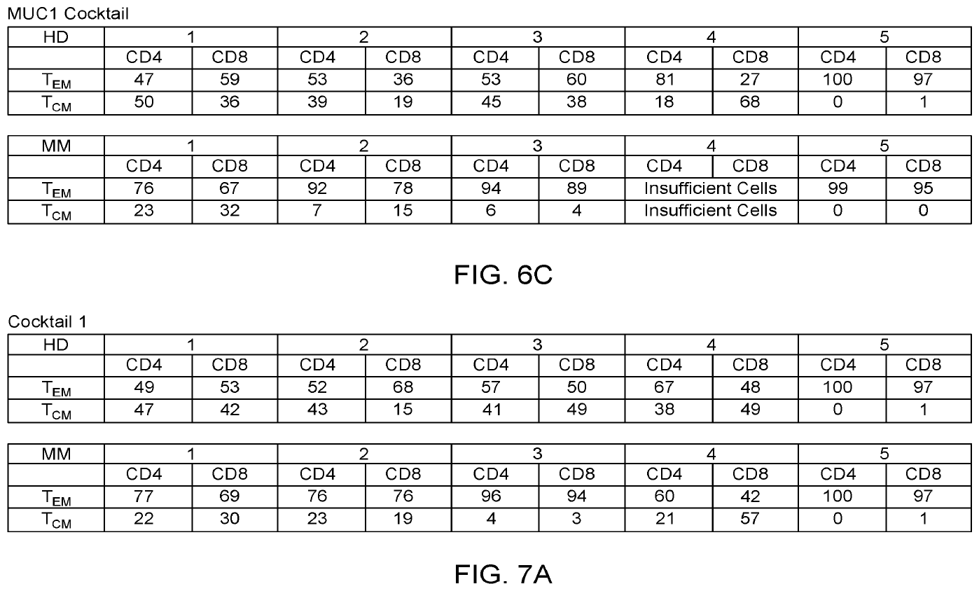

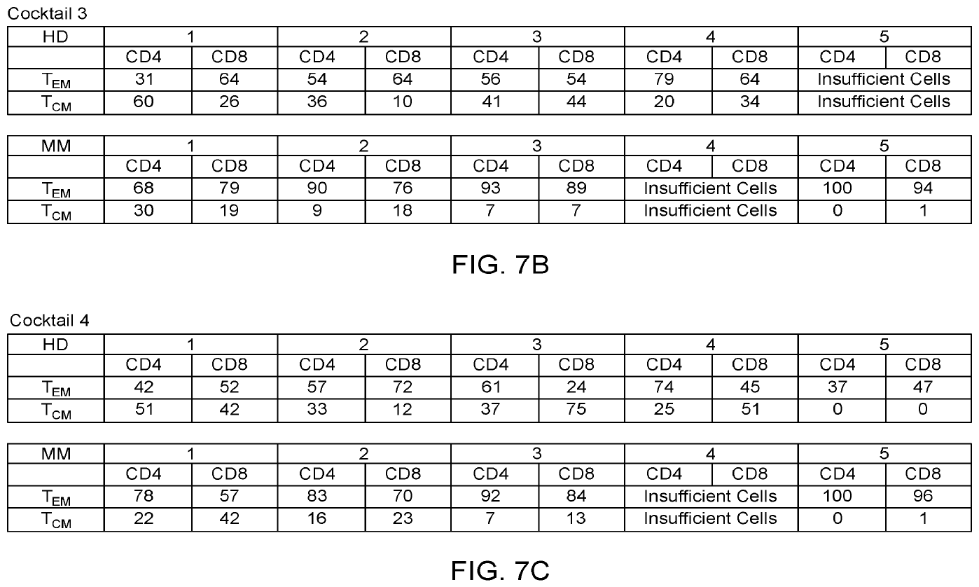

Generation of CD4⁺ and CD8⁺ effector (T_{EM}) and memory (T_{CM}) from MM patients and HDs at the end of the culture period: The T cells harvested at the end of the culture period were stained with CD62L and CD45RO for phenotypic classification. All four polypeptide cocktails generated CD4⁺ and CD8⁺ T_{EM} (CD45RO⁺CD62L⁻) and T_{CM} (CD45RO⁺CD62L⁺from PBMCs from HDs as well as MM patients (FIGS. 6A and 6B). Overall, it seems that MUC1-activated PBMCs from HDs generated CD4⁺ T_{CM} (3/5) to a greater extent than T_{EM} (2/5) whereas PBMCs from MM patients induced CD4⁺ T_{EM} to a higher level than T_{CM}. However, in the case of CD8⁺ T cells, the propensity to develop T_{EM} was greater than T_{CM} in both HDs as well as the MM patients (FIG. 6C). T_{CM} cells are more likely to survive and establish immunologic memory. Statistical analysis indicated no significant differences (Student's t-test). The data for one HD and one MM patient are shown (FIGS. 6A and 6B). FIG. 7 depicts the T_{EM} and T_{CM} observed in five different HDs and MM patients for CT1 (FIG. 7A), CT3 (FIG. 7B), and CT4 (FIG. 7C).

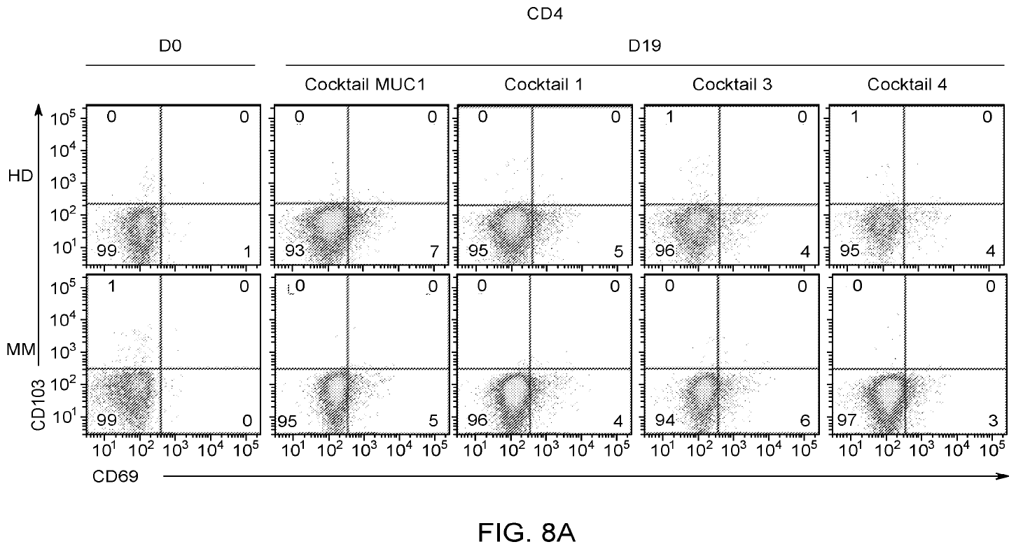

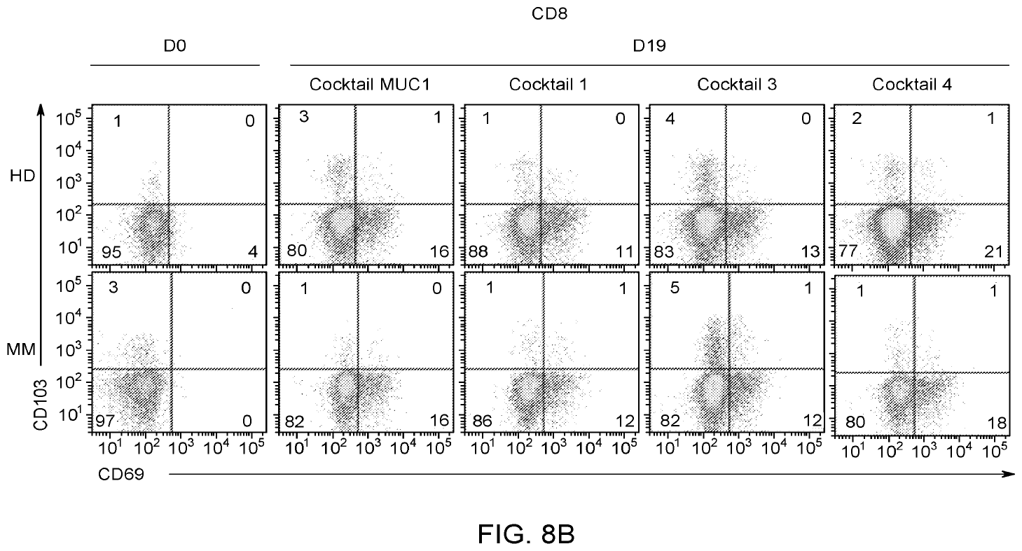

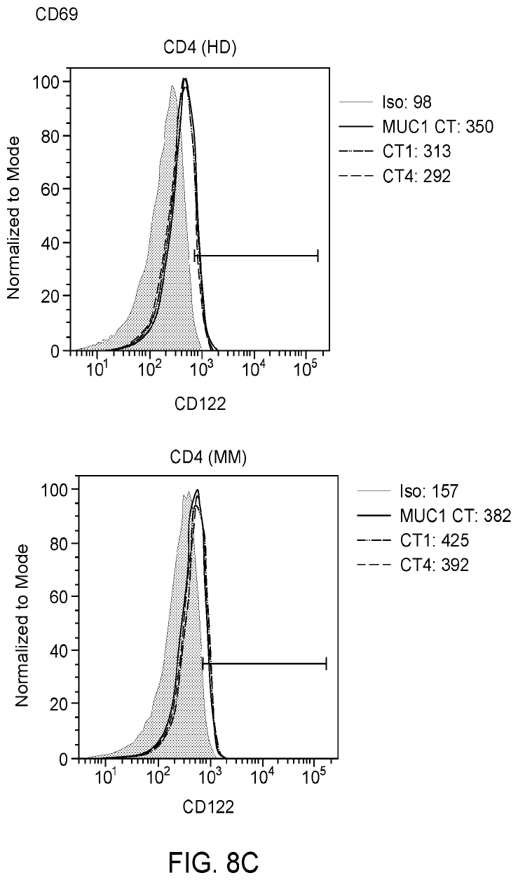

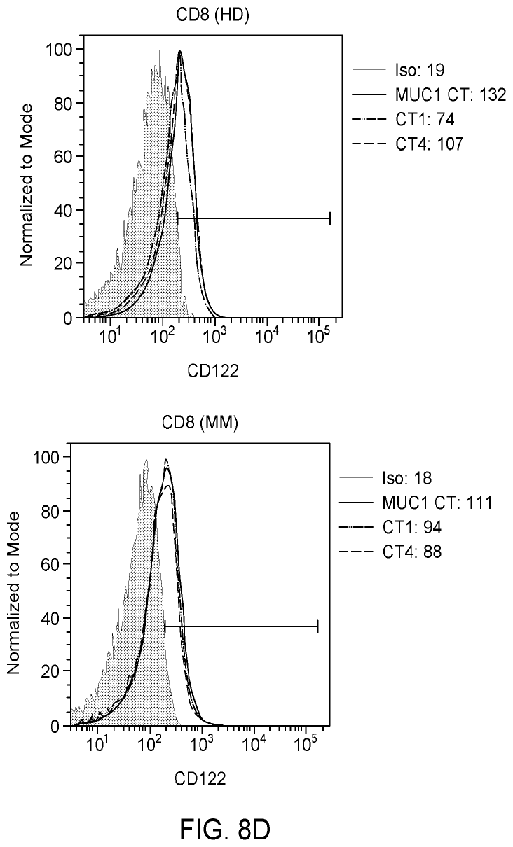

Expression of CD69 and CD103, the receptors used to delineate tissue resident memory cells (T_{RM}) was examined. CD8⁺ T cells expanded from either HD or MM PBMCs showed expression of CD69 and CD103 (FIGS. 8A and 8B). CD122 expression varied and was dependent on the polypeptide cocktail. Furthermore, both CD4⁺ and CD8⁺ T cells expressed CD122 (FIGS. 8C and 8D). However, CD122 was augmented to a greater level on CD8⁺ T cells from HD or MM patients in response to different polypeptide cocktails. The cells also showed an increase in the accumulation of neutral lipids, chemokine receptors (CD49a, CXCR6, CD101 and CXCR3) and transcription factors (Notch1).

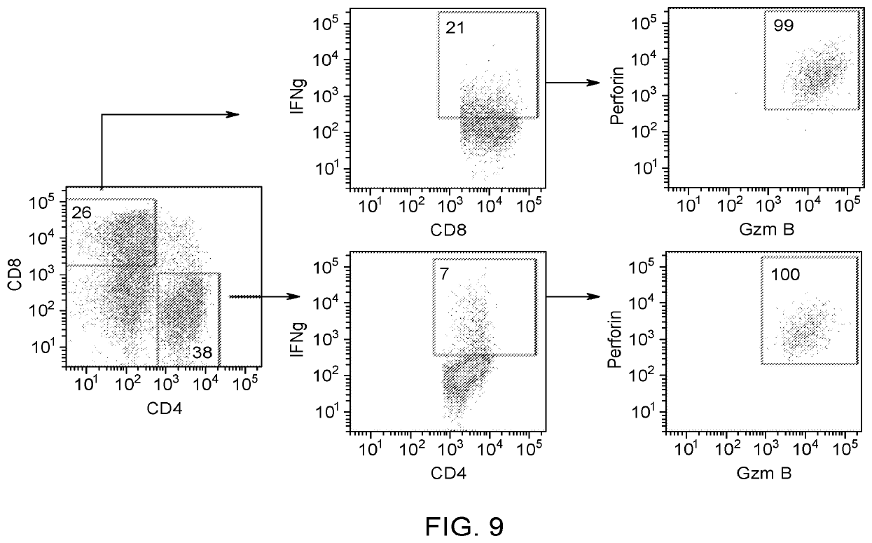

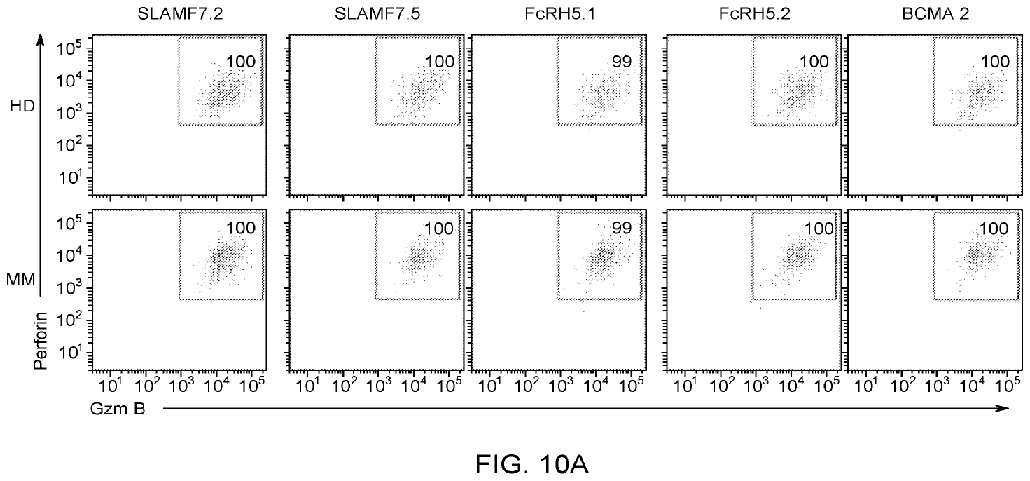

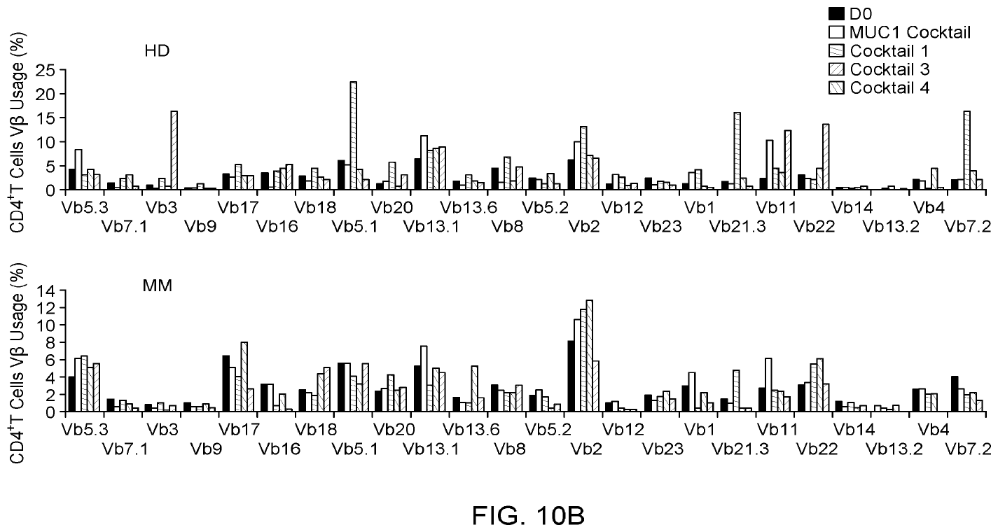

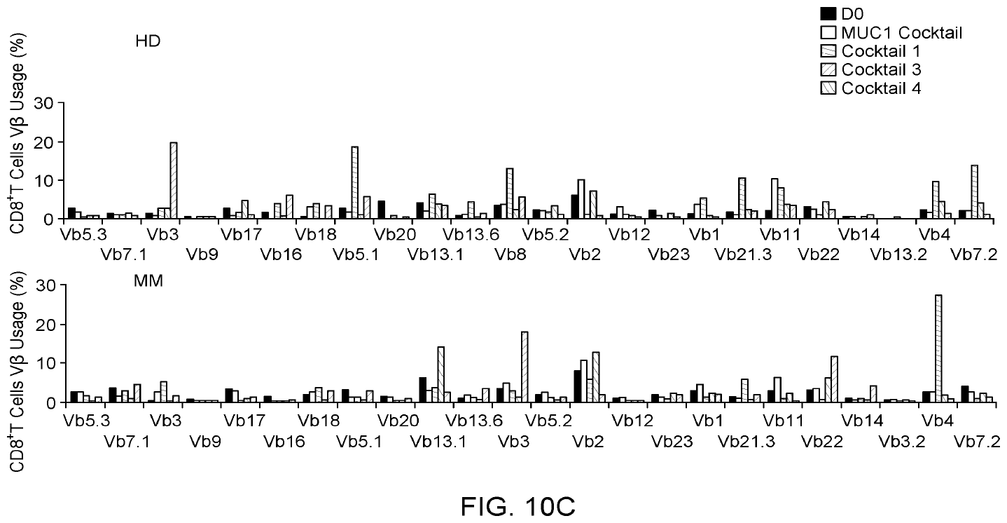

Multiclonal expansion of Ag-specific CD4⁺ and CD8⁺ T cells with cytolytic abilities: The cytolytic ability of CD4⁺ and CD8⁺ T cells was determined by examining the expression of perforin and granzyme B. The gating strategy employed is portrayed in FIG. 9. Briefly, the CD3⁺CD4⁺ and CD3⁺CD8⁺ T cells were gated on cells expressing IFN-γ, which were further analyzed for perforin and granzyme B positivity. More than 90% of the IFN-γ⁺ cells stimulated by all of the cocktails were positive both for perforin and granzyme B, proteins that are surrogates of lytic activity (FIGS. 10A and 10B).

Next TCR diversity exhibited by the naïve and activated CD4⁺ and CD8⁺ T cell populations at the initiation and commencement of the culture period was assessed. All five HDs and MM patients exhibited an increase in the number of both T cell types. Both CD4⁺ and CD8⁺ T cells exhibited multiclonal expansion regardless of the PBMC source. Some clones showed more extensive proliferation than others. A representative example (FIGS. 10B and 10C) suggests that the polypeptide cocktails successfully induced a polyclonal response with a few dominating clones in both CD4⁺ and CD8⁺ T cell compartments, regardless of the source of the PBMCs. In some cases, a monoclonal response has been observed.

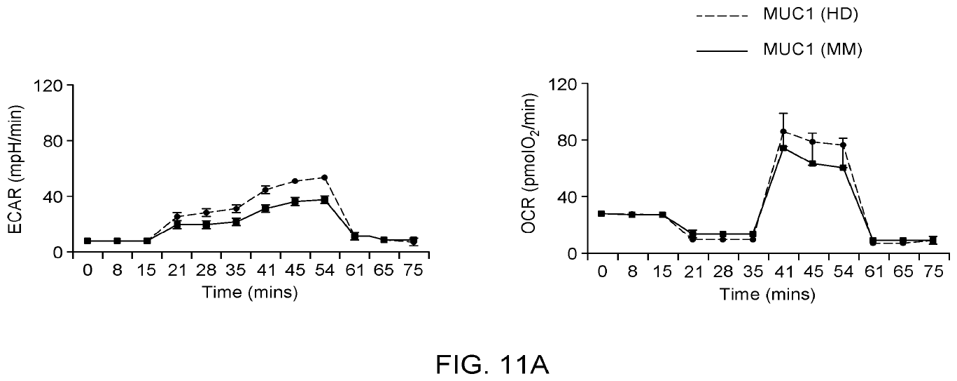

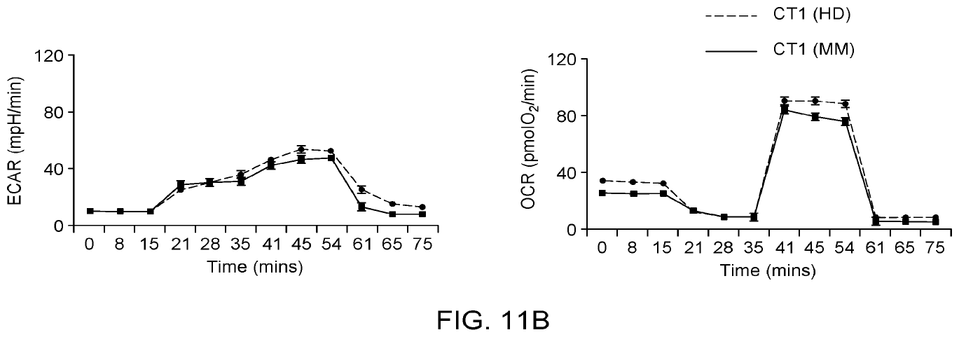

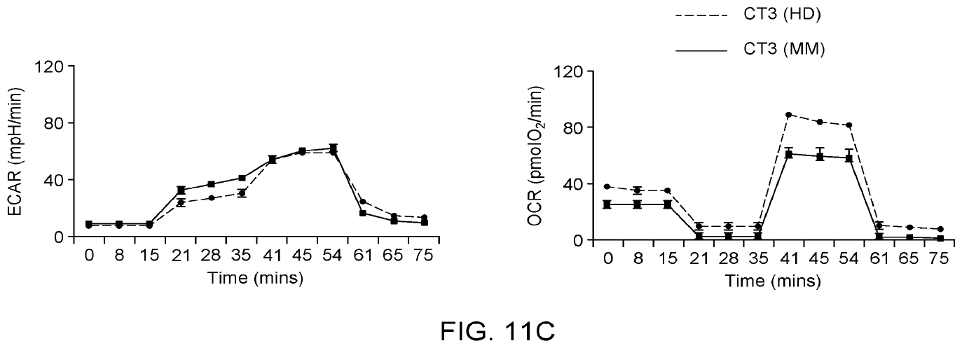

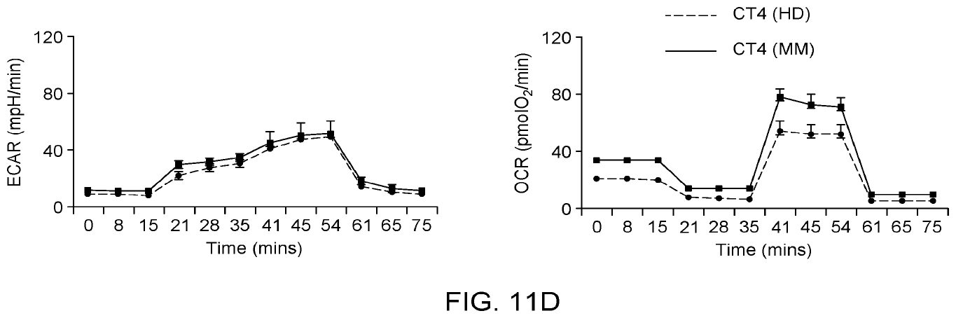

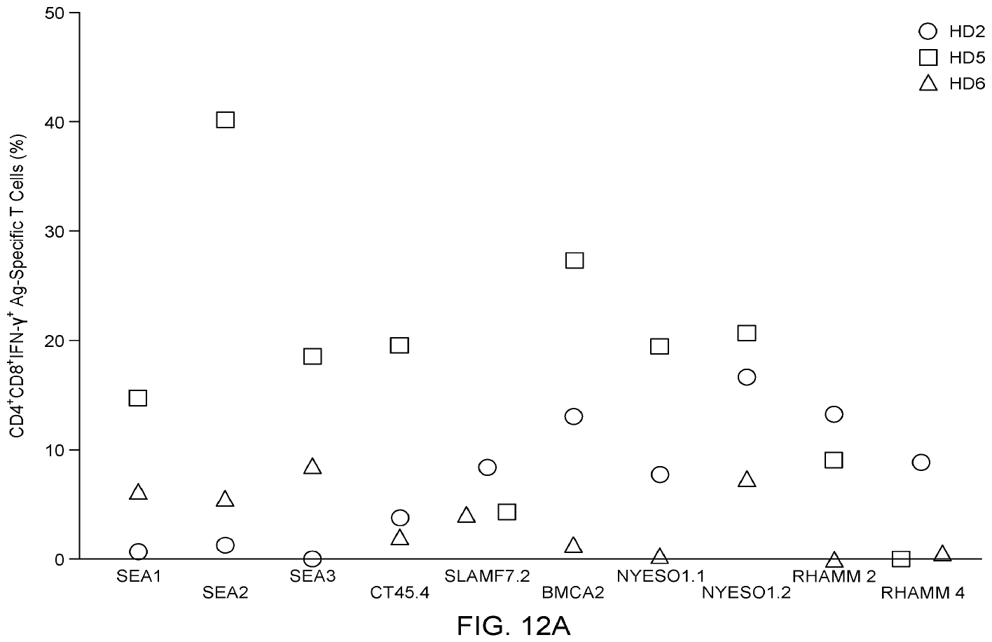

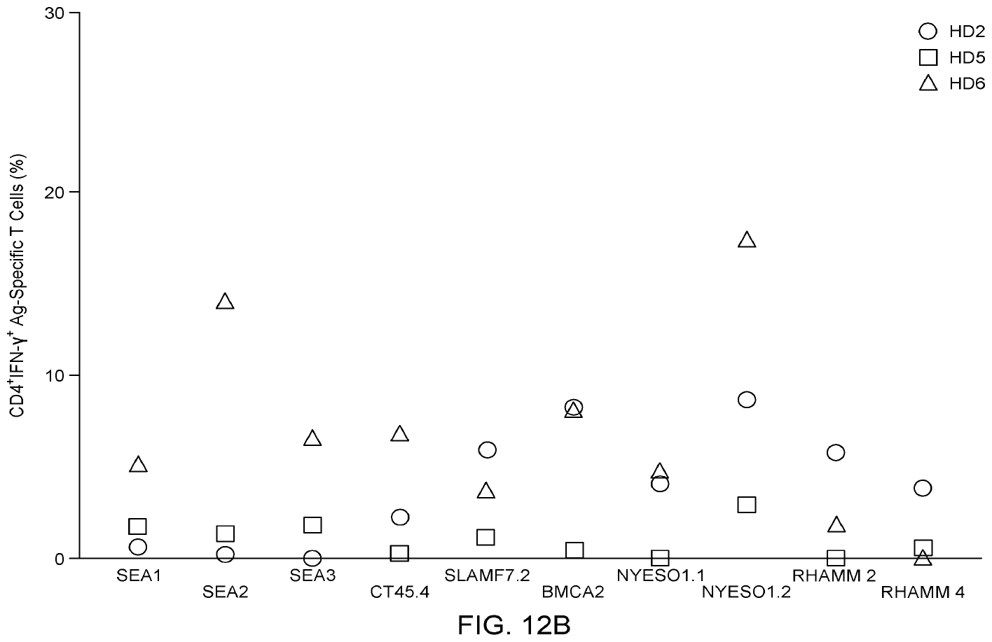

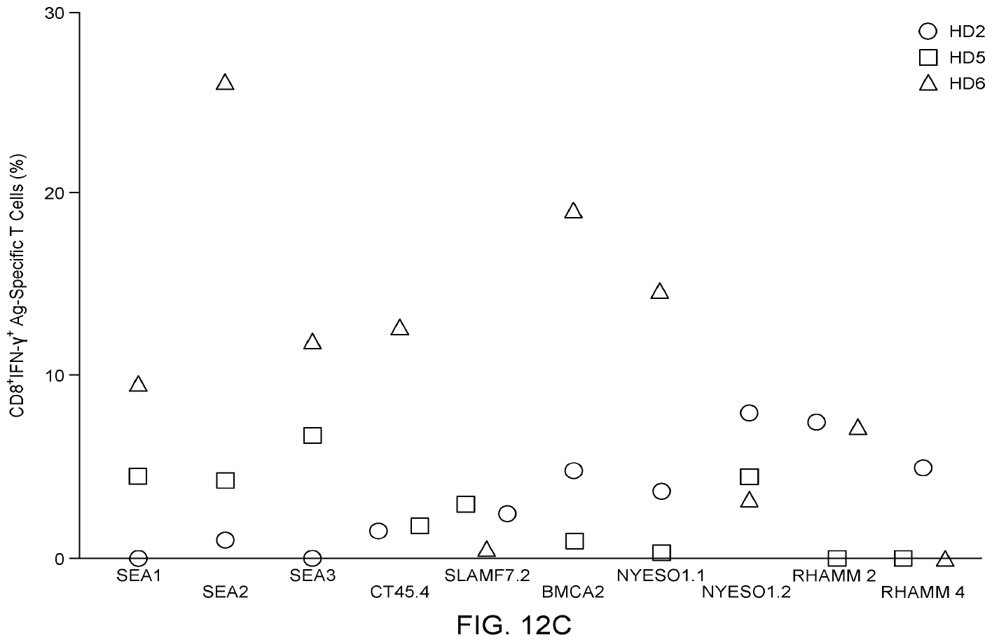

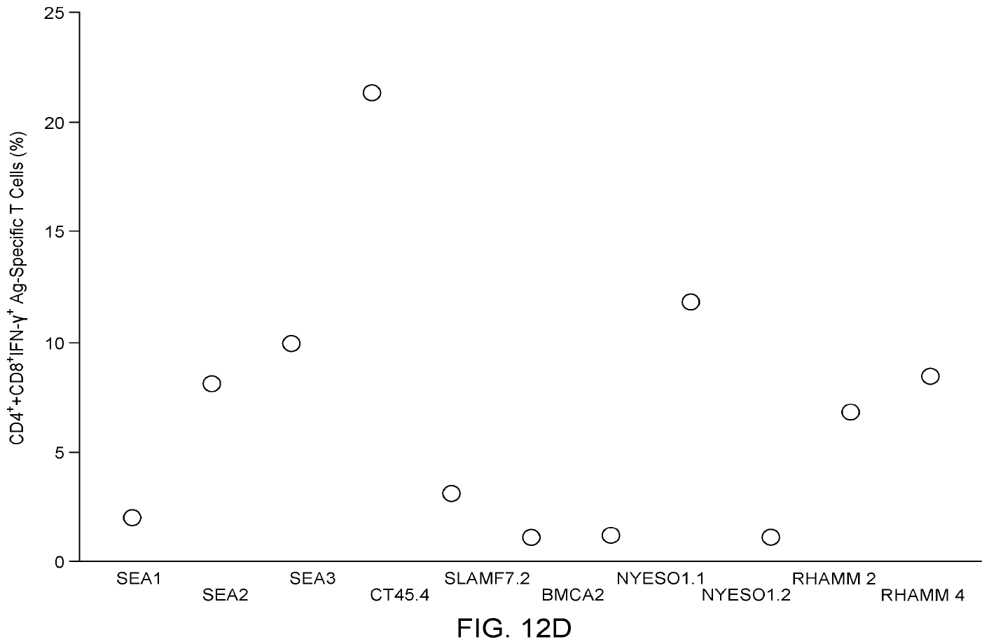

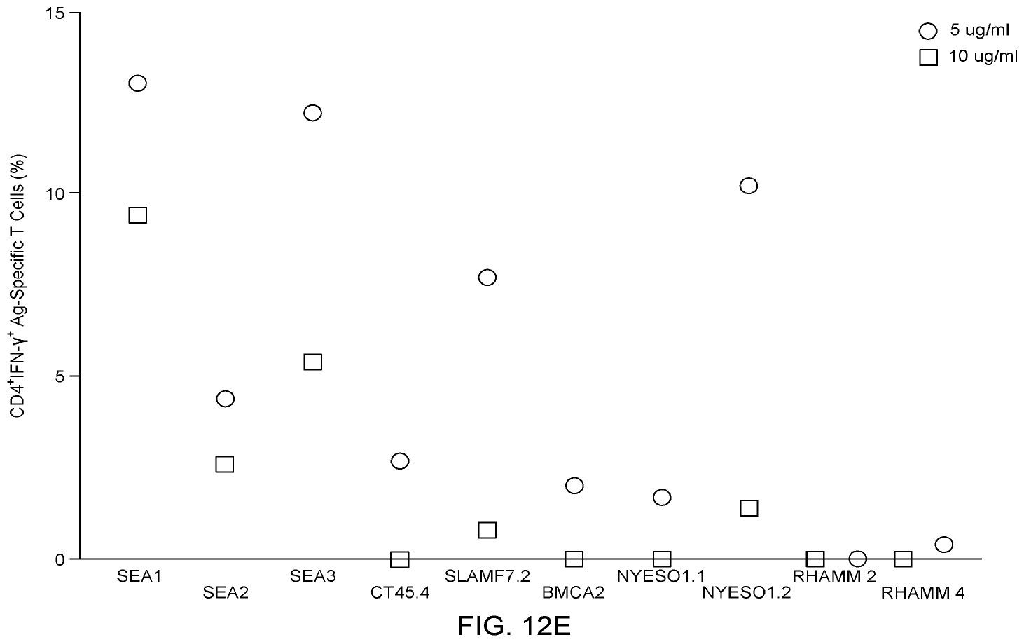

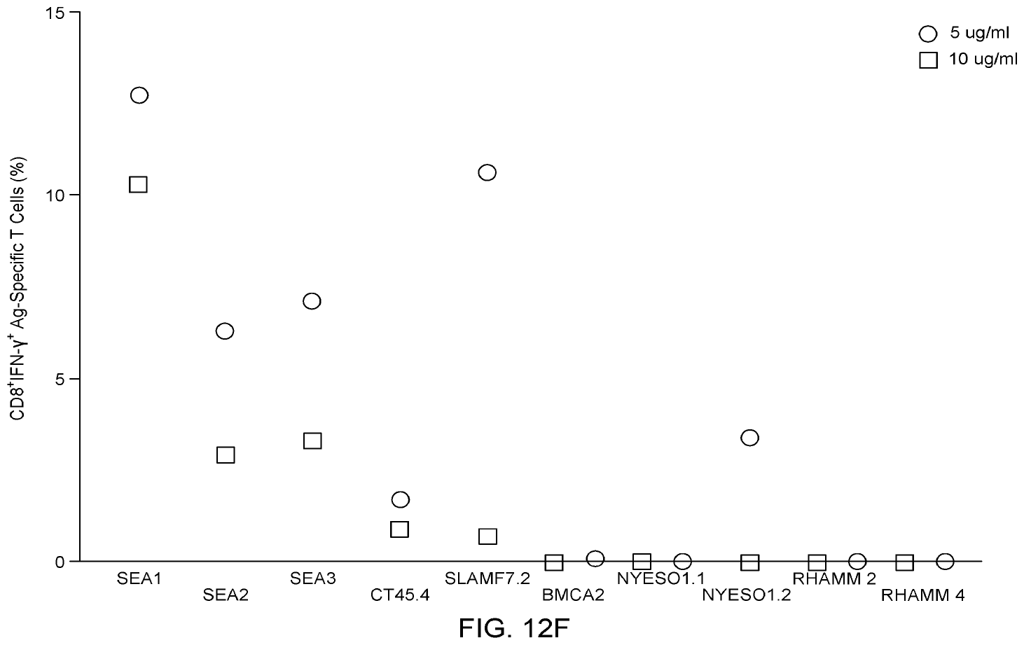

Comparative metabolic profile regardless of the source: T cells at the tumor site in MM patients have been shown to be exhausted. Increasing evidence suggests altered cellular metabolism to be one of the hallmarks of tumor cells. Furthermore, studies in the past have shown a positive association between metabolic disorder and MM incidence. To understand the metabolic profile of the culture generated T cells, the ability of PBMCs from HDs or MM patients to induce glycolysis (ECAR, extracellular acidification rate) or oxidative phosphorylation (OXPHOS, oxidative phosphorylation) was assessed. At the end of the culture period (day 19), the expanded cells following exposure to different polypeptide cocktails (MUC1 Cocktail or Cocktails 1, 3 or 4) were harvested. The metabolic profile for a representative HD and MM is depicted in FIG. 11. The rate of glycolysis was similar in cells expanded from HDs or from MM patients following stimulation with either MUC1 Cocktail (FIG. 11A, left panel), Cocktail 1 (FIG. 11B, left panel), Cocktail 3 (FIG. 11C, left panel), or Cocktail 4 (FIG. 11D, left panel). The rate of OXPHOS was also equivalent in T cells derived from PBMCs of HD compared to that from MM patients regardless of the polypeptide cocktail (FIGS. 11A-11D, right panel). Overall, the extent to which glycolysis and OXPHOS were activated in different MM PBMCs was comparable to that of HDs with no statistical differences noted (Student's t-test, p>0.1 in all comparisons). Further calculation revealed that basal respiration, ATP production, maximal respiration, spare respiratory capacity (SRC) and non-mitochondrial-derived OCR were observed to be similar in the MM-derived T cells or those from HDs, suggesting that the metabolic profile of MM-derived day 19 cells is similar to those of HD cells following polypeptide-driven stimulation in the ex vivo culture.

Together these results demonstrate that one or more polypeptides identified herein can be used to activate CD4⁺ and CD8⁺ T cells from PBMCs and generate both T_{EM} cells and T_{CM} cells. These results also suggest that activated antigen-specific T cells can be generated ex vivo from PBMCs using one or more polypeptides identified herein, and the generated antigen-specific T cells can be used in an adoptive cell transfer (ACT) to treat a mammal (e.g., the mammal from which the PBMCs were isolated).

### Materials and Methods

### Isolation and Preservation of PBMCs

The collection and preservation of HD and MM patient PBMCs were performed as described elsewhere (see, e.g., Pathangey et al., Oncotarget, 8:10785-808 (2017)). Briefly, leukapheresis was performed as per the guidelines compiled by the American Association of Blood Banks on 5 healthy volunteers with their consent. Samples were subjected to Ficoll-Hypaque density separation (Ficoll-Paque Plus, Thermo-Fisher #17-1440-02). For cancer patients, 100 mL of whole blood was collected by peripheral venipuncture which was then purified for PBMCs by the Ficoll-Hypaque density gradient centrifugation (2000 rpm for 20 minutes). For this study, PBMCs were collected from 5 MM patients at different stages of cancer. The cells were cryopreserved in liquid nitrogen using either 10% dimethyl sulfoxide (DMSO; Sigma #02650) or Cryostar CS10 (BioLife Solutions #210374).

### Patient Characteristics

MM1 has smoldering MM, with M spike increasing rapidly. No prior treatment. MM2 has amyloidosis and smoldering MM, off therapy for 6 years. Patient had received an autologous bone marrow transplantation 6 years prior. MM3 has MGUS, with type 2 diabetes. MM4 is a 70-year-old male with MM International Staging System (ISS) 2 for one year prior to blood collection. He received lenalidomide 3 weeks prior and bortezomib and dexamethasone one week prior. MM5 has untreated smoldering MM.

### Peptide Design and Synthesis

The polypeptides were mapped using open access discovery software, the NetMHCpan servers 3 and 3.2 that predicts MHC I (9 mer) & II (15 mer) binding hotspots, respectively, based on artificial neural networks. The method of designing polypeptides is as described elsewhere (see, e.g., Pathangey et al., Oncotarget, 8:10785-808 (2017)). Briefly, the Fasta sequence of the protein was submitted to NetMHCpan server 3 for determining MHC I hotspots. The alleles that were employed to detect the hotspots are as follows:
HLA-A01:01, HLA-A02:01, HLA-A03:01, HLA-A11:01, HLA-A24:02, HLA-B07:02, HLA-B08:01, HLA-B15:01, HLA-B40:01, HLA-B44:02, HLA,B51:01, HLA-C03:03, HLA-C03:04, HLA-C04:01, HLA-C05:01, HLA-C06:02, HLA-C07:01, HLA-C07:02, HLA-A23:01, HLA-A25:01, HLA-A26:01, HLA-A29:02, HLA-A32:01, HLA-B14:02, HLA-B18:01, HLA-B44:03, HLA-B53:01, HLA-B57:01, HLA-C02:02, HLA-C08:02, HLA-A30:01, HLA-A30:02, HLA-A33:03, HLA-A34:02, HLA-A68:02, HLA-A74:01, HLA-B15:03, HLA-B58:01, HLA-C16:01, HLA-B35:01, HLA-B42:01, HLA-B45:01, HLA-B49:01, HLA-C17:01, HLA-C18:01, HLA-C01:02, HLA-A31:01, HLA-A68:01, HLA-B52:01, HLA-C08:01, HLA-C12:03, HLA-A02:03, HLA-A02:06, HLA-A02:07, HLA-B13:01, HLA-B15:02, HLA-B35:01, HLA-B38:02, HLA-B40:02, HLA-B46:01, HLA-B55:02, HLA-B54:01, HLA-C03:02, HLA-C14:02

The polypeptide length was adjusted to 9 and the threshold for strong and weak binders was adjusted to 0.5 and 2.

To delineate the MHC II hotspots, NetMHCpan 3.2 was utilized. After inserting the Fasta sequence, the allele information was added, which were: DRB1_0101, DRB1_0102, DRB1_0301, DRB1_0302, DRB1_0401, DRB1_0403, DRB1_0404, DRB1_0405, DRB1_0407, DRB1_0701, DRB1_0802, DRB1_0803, DRB1_0804, DRB1_0901, DRB1_1001, DRB1_1101, DRB1_1104, DRB1_1201, DRB1_1201, DRB1_1301, DRB1_1302, DRB1_1303, DRB1_1401, DRB11404, DRB1_1406, DRB1_1501, DRB1_1502, DRB1_1503, DRB1_1602. In this case, the polypeptide length was restricted to 15 and the threshold for strong and weak binders was adjusted to 2% and 10%, respectively. The last column of the output file indicated the hotspots, which was employed to identify the sequence (MHC I-bold and italicized; MHC II-bold). The polypeptides were designed where both MHC I and II hotspots overlapped.

The designed polypeptides have high affinity for multiple class I & II haplotypes expressed in individuals across different races and ethnicities. Processing of long polypeptides (17 to 41 amino acids) was essential for activation of naive T cells.

Generation of T cells and Restimulation for Functional Analysis

PBMCs were cultured and restimulated as described elsewhere (see, e.g., Pathangey et al., Oncotarget, 8:10785-808 (2017)). Briefly, PBMCs were thawed on day 0 (D0). After washing the cells, a density of 6×10⁶ cells/mL was resuspended in AIM-V media (Gibco #0870112-DK) with 0.5% human AB serum (HuAB, Gemini Bioproducts #100-512) and 80 ng/mL GM-CSF (R & D #215-GMP-010). 0.5 mL of cell suspension was added per well in a 48-well cluster plate. On D1, the cells were stimulated (0 hours) with a single polypeptide (50 μg/mL) or a cocktail of polypeptides (25 μg/mL for each polypeptide). Resiquimod (R848, 6 μg/mL-Invivogen #vac-r848) and LPS (1 ng/mL-Invivogen #vac-3pelps) were added after 4 hours and 4.5 hours, respectively, after Ag pulsing. On D2 of culturing, the cells were detached by washing with Ca⁺²/Mg⁺²-free PBS (Gibco #10010-23) and harvested. These cells were resuspended in 6 mL of AIM-V media containing 2% of HuAB serum and 50 ng/ml of IL-7 (Miltenyl #130-095-364). 2 mL of cell suspension were added to each well of fresh 24-well cluster plates. The cells were harvested on D19 with intermittent splits on D8, D12 and D15.

A secondary stimulation was performed with the harvested T cells to analyze phenotypic and functional characteristics. For this, another PBMC vial was thawed (D17) and stimulated (D18) with each cocktail polypeptide (50 μg/mL) singly in the presence of Amphotericin B (125 ng/mL) (Lonza #17-836E). On D19, the harvested T cells and aforesaid Ag-pulsed PBMCs were co-cultured overnight at a density of 2:1, which were then used for analysis. For assessing intracellular IFN-γ, monensin (GolgiStop, BD Biosciences, San Diego CA, #554724) was added after 4-6 hours to block the export of endogenously produced cytokines. The cells were then surface stained for CD4 and CD8 followed with intracellular staining for IFN-γ.

### Metabolic Assay

Seahorse XFe bioanalyser was used to measure the Extracellular Acidification Rate (ECAR) and Oxygen Consumption Rate (OCR). The assay was performed as follows: Seahorse 96 well plates were first coated with Cell-Tak (Corning #354240) for 20 minutes. In the meantime, the cells were resuspended in Seahorse XF base DMEM media with (Agilent Technologies #103334-100) and without phenol red (Agilent Technologies #103335-100). For OCR analysis, the media contained glucose (10 mM; Sigma #G5146), sodium pyruvate (1 mM) and glutamine (2 mM) whereas for ECAR the media had only glutamine (2 mM). Day 19 cells (1.2×105) were added to each well (3 wells per sample). The cells were spun down and then placed in a non-CO₂ incubator at 37° C. for 1 hour. ECAR and OCR analyses were conducted under basal conditions and after adding the following reagents: ECAR assessment-glucose (10 mM), oligomycin (1 μM; Sigma #04867-5 mg), 2-deoxy-D-glucose 2-DG (5 mM) and for OCR-oligomycin (1 μM), p-trifluoromethoxy carbonylcyanide phenylhydrazone (FCCP) (1 μM; Sigma #C2920-10 mg), rotenone (0.5 μM; Sigma #R8875), and antimycin (5 μM; Sigma #A8674-25 mg).

### Antibodies

The following fluorochrome conjugated anti-human antibodies were used: CD3 APC efluor 780 (eBioscience #47-0036-42) or BV650 (Biolegend #317324), CD4 BV 510 (BD Horizon/BD Biosciences #562970), CD8 evolve 655 (eBioscience #86-0088-42), CD33 APC (eBioscience #17-0338-42), CD56 efluor 710 (eBioscience #46-056-42) or FITC (Biolegend #304604), PD-1 BV 785 (BioLegend #329930), CCR7 BV785 (Biolegend #353230), CD19 BV785 (Biolegend #302240), CD62L BV785 (Biolegend #304830), IFN-γ efluor 450 (eBioscience #48-7319-42), Perforin Alexa Fluor 647 (Biolegend #353322) or PE (Biolegend #353304), granzyme FITC (Biolegend #515403). Appropriate isotype control antibodies were employed to determine the specificity of test antibodies.

### Multiparameter Flow Cytometry

Cells were centrifuged and stained for 30 minutes at RT with Live/Dead UV Blue stain (Life Technologies, #L23105; diluted 1:1000 in PBS) for assessing viability. After washing, cells were exposed to Fc receptor block (50 μg of unconjugated human IgG; Sigma Aldrich #S-8032) and stained for surface proteins (30 minutes at 4° C.) by adding respective antibodies in FACs buffer. The FACS buffer is Ca⁺²/Mg⁺²-free PBS with 1% heat-inactivated fetal bovine serum (Sigma Aldrich #F2442) and 0.02% sodium azide (Sigma Aldrich #S-8032). For analyzing intracellular proteins, the cells were then subjected to fixation and permeabilization according to the manufacturer's guidelines (eBioscience, San Diego CA, #00-5123-43, #00-5223-56 and #00-8333-56; BD Biosciences #51-2090KZ, #51-2091KZ) followed by staining with appropriate antibodies. Flow cytometry data was acquired on Fortessa (BD Bioscience) and analyzed with FACSDiva software (BD Biosciences) or Flowjo.

### Vβ Frequency Analysis

The Vβ repertoire of CD3⁺CD4⁺ and CD3⁺CD8⁺ T cells was assessed by manufacturer's protocol with the kit—IOTest Beta Mark (Beckman Coulter). Antibodies detect only about 70% of the T cell receptor (TCR) Vβ repertoire.

### Statistical Analysis

To compare the means of each of the groups or between HDs and MM patients, a two-sided Student's t-test was used. p≤0.05 was considered statistically significant.

### Abbreviations

ACT—Adoptive Cell Transfer
Ag—Antigen
BCMA—B Cell maturation Antigen
CAR-T—Chimeric Antigen Receptor T Cells
CD3/4/8/33/56/19—Cluster of Differentiation 3/4/8/33/56/19
CRS—Cytokine Release Syndrome
CTAs—Cancer Testis Antigens
CT1—Cocktail 1
CT3—Cocktail 3
CT4—Cocktail 4
CT45—Cancer Testis Antigen Family 45
2-DG—2-Deoxy-D-Glucose
D0/1/2/17/19-Day 0/1/2/17/19
ECAR—Extracellular Acidification Rate
FcRH5—Fc Receptor Like 5
FCCP—Carbonyl Cyanide p-Trifluoromethoxyphenylhydrazone
GM-CSF—Granulocyte-Macrophage Colony-Stimulating Factor
HD—Healthy Donor
H—Hour
IFN-γ—Interferon-Gamma
IL-7—Interleukin-7
KLRG1—Killer Cell Lectin-Like Receptor G1
LPS—Lipopolysaccharide
MAGEA3/6—Melanoma Antigen Family A3/6
MCL1—Myeloid Cell Leukemia 1
MHC I—Major Histocompatibility Complex I
MHC II—Major Histocompatability Complex II
MM—Multiple Myeloma
MUC1—Mucin 1
MUC1 CT—Mucin 1 Cocktail
NK cells—Natural Killer Cells
NY-ESO-1—New York Esophageal Squamous Cell Carcinoma 1
OCR—Oxygen Consumption Rate
OXPHOS—Oxidative Phosphorylation
PBMC—Peripheral Blood Mononuclear Cells
RHAMM—Receptor for Hyaluronan-Mediated Motility
SEA polypeptides—derived from Sperm Protein, Enterokinase and Agrin domain in MUC1 SEPT9—SEPTIN9
SLAMF7-Self-Ligand Receptor of the Signaling Lymphocytic Activation Molecule Family 7
SRC—Spare Respiratory Capacity
TCR—T Cell Receptor
TLR—Toll-Like Receptor
T_{EM}—T Effector Memory Cells
T_{CM}—T Central Memory Cells
T_{RM}—T Resident Memory Cells
T_{reg}—Regulatory T cells
Vβ—Variable Beta
WT1—Wilms Tumor 1
XBP(S)1—Spliced isoform of X-Box Binding Protein 1

### Example 2: Treating Cancer

PBMCs are obtained from a human having MM. The obtained PBMCs are contacted with one or more polypeptides provided herein (e.g., a composition that contains one or more polypeptides provided herein) are cultured with the PBMCs to activate T cells within that cell population and generate antigen-specific T cells (e.g., antigen-specific CD4⁺ and/or antigen-specific CD8⁺ T_{EM} cells and/or antigen-specific CD4⁺ and/or antigen-specific CD8⁺ T_{CM} cells) that can target (e.g., target and destroy) MM cancer cells expressing the one or more polypeptides.

The activated antigen-specific T cells are administered to the human having MM to treat the mammal.

### Example 3: Treating Cancer

PBMCs are obtained from a healthy human donor. The obtained PBMCs are contacted with one or more polypeptides provided herein (e.g., a composition that contains one or more polypeptides provided herein) and cultured with the PBMCs to activate T cells within that cell population and to generate antigen-specific T cells (e.g., antigen-specific CD4⁺ and/or antigen-specific CD8⁺ T_{EM} cells and/or antigen-specific CD4⁺ and/or antigen-specific CD8⁺ T_{CM} cells) that can target (e.g., target and destroy) MM cancer cells expressing the one or more polypeptides.

The activated antigen-specific T cells are administered to a human having MM to treat the mammal.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An isolated polypeptide consisting of the amino acid sequence set forth in any one of SEQ ID NOs: 1-364.

2. A composition comprising an isolated polypeptide of claim 1.

3. A composition comprising at least two polypeptides, wherein each of said at least two polypeptides is a polypeptide consisting of the amino acid sequence set forth in any one of SEQ ID NOs: 1-364.

4. The composition of claim 2, wherein said composition comprises a polypeptide consisting of the amino acid sequence set forth in SEQ ID NO: 1, a polypeptide consisting of the amino acid sequence set forth in SEQ ID NO:2, and a polypeptide consisting of the amino acid sequence set forth in SEQ ID NO:3.

5. The composition of claim 2, wherein said composition comprises a polypeptide consisting of the amino acid sequence set forth in SEQ ID NO: 1, a polypeptide consisting of the amino acid sequence set forth in SEQ ID NO:20, a polypeptide consisting of the amino acid sequence set forth in SEQ ID NO:23, a polypeptide consisting of the amino acid sequence set forth in SEQ ID NO:24, and a polypeptide consisting of the amino acid sequence set forth in SEQ ID NO:31.

6. The composition of claim 2, wherein said composition comprises a polypeptide consisting of the amino acid sequence set forth in SEQ ID NO: 4, a polypeptide consisting of the amino acid sequence set forth in SEQ ID NO:7, a polypeptide consisting of the amino acid sequence set forth in SEQ ID NO:8, a polypeptide consisting of the amino acid sequence set forth in SEQ ID NO:13, and a polypeptide consisting of the amino acid sequence set forth in SEQ ID NO:14.

7. The composition of claim 2, wherein said composition comprises a polypeptide consisting of the amino acid sequence set forth in SEQ ID NO: 10, a polypeptide consisting of the amino acid sequence set forth in SEQ ID NO:11, a polypeptide consisting of the amino acid sequence set forth in SEQ ID NO: 12, a polypeptide consisting of the amino acid sequence set forth in SEQ ID NO:28, and a polypeptide consisting of the amino acid sequence set forth in SEQ ID NO:29.

8. The composition of claim 2, wherein said composition comprises a polypeptide consisting of the amino acid sequence set forth in SEQ ID NO: 1, a polypeptide consisting of the amino acid sequence set forth in SEQ ID NO:2, a polypeptide consisting of the amino acid sequence set forth in SEQ ID NO:3, a polypeptide consisting of the amino acid sequence set forth in SEQ ID NO:4, a polypeptide consisting of the amino acid sequence set forth in SEQ ID NO: 10, a polypeptide consisting of the amino acid sequence set forth in SEQ ID NO: 12, a polypeptide consisting of the amino acid sequence set forth in SEQ ID NO:13, a polypeptide consisting of the amino acid sequence set forth in SEQ ID NO: 20, a polypeptide consisting of the amino acid sequence set forth in SEQ ID NO:28, a polypeptide consisting of the amino acid sequence set forth in SEQ ID NO:29 and a polypeptide consisting of the amino acid sequence set forth in SEQ ID NO:31.

9. The composition of claim 2, wherein said composition comprises a polypeptide consisting of the amino acid sequence set forth in SEQ ID NO: 1, a polypeptide consisting of the amino acid sequence set forth in SEQ ID NO:2, a polypeptide consisting of the amino acid sequence set forth in SEQ ID NO:3, a polypeptide consisting of the amino acid sequence set forth in SEQ ID NO:4, a polypeptide consisting of the amino acid sequence set forth in SEQ ID NO:10, a polypeptide consisting of the amino acid sequence set forth in SEQ ID NO:12, a polypeptide consisting of the amino acid sequence set forth in SEQ ID NO:13, a polypeptide consisting of the amino acid sequence set forth in SEQ ID NO: 20, a polypeptide consisting of the amino acid sequence set forth in SEQ ID NO:23, a polypeptide consisting of the amino acid sequence set forth in SEQ ID NO:28, a polypeptide consisting of the amino acid sequence set forth in SEQ ID NO:29, and a polypeptide consisting of the amino acid sequence set forth in SEQ ID NO:32.

10. The composition of claim 2, wherein said composition comprises a polypeptide consisting of the amino acid sequence set forth in SEQ ID NO: 2, a polypeptide consisting of the amino acid sequence set forth in SEQ ID NO:4, a polypeptide consisting of the amino acid sequence set forth in SEQ ID NO: 12, a polypeptide consisting of the amino acid sequence set forth in SEQ ID NO:13, and a polypeptide consisting of the amino acid sequence set forth in SEQ ID NO:29.

11. The composition of claim 2, wherein said composition comprises a polypeptide consisting of the amino acid sequence set forth in SEQ ID NO: 1, a polypeptide consisting of the amino acid sequence set forth in SEQ ID NO:10, and a polypeptide consisting of the amino acid sequence set forth in SEQ ID NO:20.

12. The composition of claim 2, wherein said composition comprises a polypeptide consisting of the amino acid sequence set forth in SEQ ID NO: 2, a polypeptide consisting of the amino acid sequence set forth in SEQ ID NO:4, and a polypeptide consisting of the amino acid sequence set forth in SEQ ID NO:29.

13. The composition of claim 2, wherein said composition comprises a polypeptide consisting of the amino acid sequence set forth in SEQ ID NO: 3, a polypeptide consisting of the amino acid sequence set forth in SEQ ID NO:12, and a polypeptide consisting of the amino acid sequence set forth in SEQ ID NO:13.

14. The composition of claim 2, wherein said composition comprises a polypeptide consisting of the amino acid sequence set forth in SEQ ID NO: 11, a polypeptide consisting of the amino acid sequence set forth in SEQ ID NO:14, and a polypeptide consisting of the amino acid sequence set forth in SEQ ID NO:28.

15. The composition of claim 2, wherein said composition comprises a polypeptide consisting of the amino acid sequence set forth in SEQ ID NO: 28 and a polypeptide consisting of the amino acid sequence set forth in SEQ ID NO:29.

16. The composition of claim 2, wherein said composition comprises a polypeptide consisting of the amino acid sequence set forth in SEQ ID NO: 13 and a polypeptide consisting of the amino acid sequence set forth in SEQ ID NO:14.

17. The composition of claim 2, wherein said composition comprises a polypeptide consisting of the amino acid sequence set forth in SEQ ID NO: 19 and a polypeptide consisting of the amino acid sequence set forth in SEQ ID NO:20.

18. The composition of claim 2, wherein said composition comprises a polypeptide consisting of the amino acid sequence set forth in SEQ ID NO: 10, a polypeptide consisting of the amino acid sequence set forth in SEQ ID NO:11, and a polypeptide consisting of the amino acid sequence set forth in SEQ ID NO:12.

19. A method for activating T cells having specificity for a cancer antigen, wherein said method comprises contacting a cell population comprising T cells with at least one polypeptide consisting of the amino acid sequence set forth in any one of SEQ ID NOs: 1-364.

20. The method of claim 19, wherein said cell population comprises unfractionated PBMCs.

21. The method of claim 19, wherein the cells of said cell population are human cells.

22. The method of claim 19, wherein said contacting is performed in vitro.

23. A method of treating a mammal having cancer or a precancerous condition, wherein said method comprises contacting T cells with at least one polypeptide consisting of the amino acid sequence set forth in any one of SEQ ID NOs: 1-364 to activate said T cells, and administering said activated T cells to said mammal.

24. The method of claim 23, wherein said mammal is a human.

25. The method of claim 23, wherein said T cell are obtained from said mammal.

26. The method of claim 23, wherein said mammal has said cancer, and wherein said administering reduces the number of cancer cells within said mammal.

27. The method of claim 26, wherein said cancer is selected from the group consisting of MM, colorectal cancer, breast cancer, non-Hodgkin's lymphoma, and ovary cancer.

28. The method of claim 23, wherein said mammal has said precancerous condition, and wherein said administering reduces a symptom of said precancerous condition within said mammal.

29. The method of claim 28, wherein said precancerous condition is MGUS.

30. The method of claim 23, further comprising expanding said activated T-cells prior to administering said activated T-cells to said mammal.

31-34. (canceled)


SEQUENCE LISTING (365 sequences)

SEQ ID NO: 1 - protein, 32 aa, synthetic construct
  REGION 1..32: note = polypeptide that can be used to activate T cells
SPQLSTGVSFFFLSFHISNLQFNSSLEDPSTD

SEQ ID NO: 2 - protein, 39 aa, synthetic construct
  REGION 1..39: note = polypeptide that can be used to activate T cells
STDYYQELQRDISEMFLQIYKQGGFLGLSNIKFRPGSVV

SEQ ID NO: 3 - protein, 32 aa, synthetic construct
  REGION 1..32: note = polypeptide that can be used to activate T cells
DVETQFNQYKTEAASRYNLTISDVSVSDVPFP

SEQ ID NO: 4 - protein, 37 aa, synthetic construct
  REGION 1..37: note = polypeptide that can be used to activate T cells
FPLPAMEEGATILVTTKTNDYCKSLPAALSATEIEKS

SEQ ID NO: 5 - protein, 26 aa, synthetic construct
  REGION 1..26: note = polypeptide that can be used to activate T cells
YQSCPDWRKDCSNNPVSVFWKTVSRR

SEQ ID NO: 6 - protein, 26 aa, synthetic construct
  REGION 1..26: note = polypeptide that can be used to activate T cells
ACDVVHVMLNGSRSKIFDKNSTFGSV

SEQ ID NO: 7 - protein, 38 aa, synthetic construct
  REGION 1..38: note = polypeptide that can be used to activate T cells
CTADNGFGPQRSEVVSLFVTVPVSRPILTLRVPRAQAV

SEQ ID NO: 8 - protein, 29 aa, synthetic construct
  REGION 1..29: note = polypeptide that can be used to activate T cells
AQHSDTISLSVIVPVSRPILTFRAPRAQA

SEQ ID NO: 9 - protein, 29 aa, synthetic construct
  REGION 1..29: note = polypeptide that can be used to activate T cells
GAEVPDVTATPARLLFFAPTRRAAPLEEM

SEQ ID NO: 10 - protein, 36 aa, synthetic construct
  REGION 1..36: note = polypeptide that can be used to activate T cells
RLNAALREKTSLSANNATLEKQLIELTRTNELLKSK

SEQ ID NO: 11 - protein, 38 aa, synthetic construct
  REGION 1..38: note = polypeptide that can be used to activate T cells
SENGNQKNLRILSLELMKLRNKRETKMRGMMAKQEGME

SEQ ID NO: 12 - protein, 32 aa, synthetic construct
  REGION 1..32: note = polypeptide that can be used to activate T cells
AHTQATLLLQEKYDSMVQSLEDVTAQFESYKA

SEQ ID NO: 13 - protein, 40 aa, synthetic construct
  REGION 1..40: note = polypeptide that can be used to activate T cells
DVIYTWKALGQAANESHNGSILPISWRWGESDMTFICVAR

SEQ ID NO: 14 - protein, 28 aa, synthetic construct
  REGION 1..28: note = polypeptide that can be used to activate T cells
LGLFLWFLKRERQEENNPKGRSSKYGLL

SEQ ID NO: 15 - protein, 38 aa, synthetic construct
  REGION 1..38: note = polypeptide that can be used to activate T cells
NSPDRVKRPMNAFMVWSRGQRRKMAQENPKMHNSEISK

SEQ ID NO: 16 - protein, 38 aa, synthetic construct
  REGION 1..38: note = polypeptide that can be used to activate T cells
EWKLLSETEKRPFIDEAKRLRALHMKEHPDYKYRPRRK

SEQ ID NO: 17 - protein, 30 aa, synthetic construct
  REGION 1..30: note = polypeptide that can be used to activate T cells
AAAPNPADGTPKVYYPSGQPASAAGAPAGQ

SEQ ID NO: 18 - protein, 38 aa, synthetic construct
  REGION 1..38: note = polypeptide that can be used to activate T cells
SDILLGILDNLDPVMFFKCPSPEPASLEELPEVYPEGP

SEQ ID NO: 19 - protein, 31 aa, synthetic construct
  REGION 1..31: note = polypeptide that can be used to activate T cells
AVDPETVFKRPRECDSPSYQKRQRMALLARK

SEQ ID NO: 20 - protein, 34 aa, synthetic construct
  REGION 1..34: note = polypeptide that can be used to activate T cells
LEGVQGPTAVRKRFFESIIKEAARCMRRDFVKHL

SEQ ID NO: 21 - protein, 27 aa, synthetic construct
  REGION 1..27: note = polypeptide that can be used to activate T cells
TFPDLESEFQAALSRKVAELVHFLLLK

SEQ ID NO: 22 - protein, 31 aa, synthetic construct
  REGION 1..31: note = polypeptide that can be used to activate T cells
PDAESLFREALSNKVDELAHFLLRKYRAKEL

SEQ ID NO: 23 - protein, 27 aa, synthetic construct
  REGION 1..27: note = polypeptide that can be used to activate T cells
TFPDLESEFQAALSRKVAKLVHFLLLK

SEQ ID NO: 24 - protein, 41 aa, synthetic construct
  REGION 1..41: note = polypeptide that can be used to activate T cells
FVQENYLEYRQVPGSDPACYEFLWGPRALIETSYVKVLHHM

SEQ ID NO: 25 - protein, 28 aa, synthetic construct
  REGION 1..28: note = polypeptide that can be used to activate T cells
SPLQRPVSSFFSYTLASLLQSSHESPQS

SEQ ID NO: 26 - protein, 25 aa, synthetic construct
  REGION 1..25: note = polypeptide that can be used to activate T cells
SSTSSSLSKSSPESPLQSPVISFSS

SEQ ID NO: 27 - protein, 37 aa, synthetic construct
  REGION 1..37: note = polypeptide that can be used to activate T cells
NSSPPRYEFLWGPRAHSEVIKRKVVEFLAMLKNTVPI

SEQ ID NO: 28 - protein, 37 aa, synthetic construct
  REGION 1..37: note = polypeptide that can be used to activate T cells
ARGPESRLLEFYLAMPFATPMEAELARRSLAQDAPPL

SEQ ID NO: 29 - protein, 28 aa, synthetic construct
  REGION 1..28: note = polypeptide that can be used to activate T cells
VPGVLLKEFTVSGNILTIRLTAADHRQL

SEQ ID NO: 30 - protein, 32 aa, synthetic construct
  REGION 1..32: note = polypeptide that can be used to activate T cells
RSASETSEKRPFMCAYPGCNKRYFKLSHLQMH

SEQ ID NO: 31 - protein, 35 aa, synthetic construct
  REGION 1..35: note = polypeptide that can be used to activate T cells
DTRVHCCLYFIPATGHSLRPLDIEFMKRLSKVVNI

SEQ ID NO: 32 - protein, 29 aa, synthetic construct
  REGION 1..29: note = polypeptide that can be used to activate T cells
LVNEKFREMIPFAVVGSDHEYQVNGKRIL

SEQ ID NO: 33 - protein, 34 aa, synthetic construct
  REGION 1..34: note = polypeptide that can be used to activate T cells
TTHCEFAYLRDLLIRTHMQNIKDITSSIHFEAYR

SEQ ID NO: 34 - protein, 30 aa, synthetic construct
  REGION 1..30: note = polypeptide that can be used to activate T cells
QLSTGVSFFFLSFHISNLQFNSSLEDPSTD

SEQ ID NO: 35 - protein, 28 aa, synthetic construct
  REGION 1..28: note = polypeptide that can be used to activate T cells
SPQLSTGVSFFFLSFHISNLQFNSSLED

SEQ ID NO: 36 - protein, 31 aa, synthetic construct
  REGION 1..31: note = polypeptide that can be used to activate T cells
SPQLSTGVSFFFLTFHLSNLNFNSSLEDPST

SEQ ID NO: 37 - protein, 32 aa, synthetic construct
  REGION 1..32: note = polypeptide that can be used to activate T cells
SVQISTGVSFFFLSFHISNLQFNSSLEDPSTD

SEQ ID NO: 38 - protein, 31 aa, synthetic construct
  REGION 1..31: note = polypeptide that can be used to activate T cells
PQLYTGVSFFFLSFHISNLQFNSSLEDPSTD

SEQ ID NO: 39 - protein, 32 aa, synthetic construct
  REGION 1..32: note = polypeptide that can be used to activate T cells
SPQLSTGVSFFMLSFHISNLQFNPSLEDPSTD

SEQ ID NO: 40 - protein, 32 aa, synthetic construct
  REGION 1..32: note = polypeptide that can be used to activate T cells
SPQLSTGVSFFFLSFHISNLQFNSSLADPSTD

SEQ ID NO: 41 - protein, 32 aa, synthetic construct
  REGION 1..32: note = polypeptide that can be used to activate T cells
SPVQLSTGVSFFFLSFHISNLQFNPSLEDPST

SEQ ID NO: 42 - protein, 32 aa, synthetic construct
  REGION 1..32: note = polypeptide that can be used to activate T cells
SPQLSTGVSFFFLYFHISNLQFNSSLEDPSTD

SEQ ID NO: 43 - protein, 32 aa, synthetic construct
  REGION 1..32: note = polypeptide that can be used to activate T cells
SPQLSTGVSFFFLTFHISNLQFNSSLPDTSTD

SEQ ID NO: 44 - protein, 31 aa, synthetic construct
  REGION 1..31: note = polypeptide that can be used to activate T cells
PQFSTGVSFFFLTFHISNLQFNSSLADPSTD

SEQ ID NO: 45 - protein, 37 aa, synthetic construct
  REGION 1..37: note = polypeptide that can be used to activate T cells
STDYYQELQRDISEMFLQIYKQGGFLGLSNIKFRPGS

SEQ ID NO: 46 - protein, 39 aa, synthetic construct
  REGION 1..39: note = polypeptide that can be used to activate T cells
STDYYQALQRDISEMFLQIYKQGGFLGLSNIKFRPGSVV

SEQ ID NO: 47 - protein, 40 aa, synthetic construct
  REGION 1..40: note = polypeptide that can be used to activate T cells
STSDYYQELQRDISNMFLQIYKQGGMLGLSNIKFRIGSVA

SEQ ID NO: 48 - protein, 39 aa, synthetic construct; identical to SEQ ID NO: 46
  REGION 1..39: note = polypeptide that can be used to activate T cells

SEQ ID NO: 49 - protein, 39 aa, synthetic construct
  REGION 1..39: note = polypeptide that can be used to activate T cells
STDYYQELQRDISEMYLQIYKQTGFLGLSNIKFRPGSVV

SEQ ID NO: 50 - protein, 39 aa, synthetic construct
  REGION 1..39: note = polypeptide that can be used to activate T cells
STDYYQELQRDISYMFLQIYKQGGFLGLSNIKMRPGTVV

SEQ ID NO: 51 - protein, 37 aa, synthetic construct
  REGION 1..37: note = polypeptide that can be used to activate T cells
DYYQELQRDISEMFLQIYKQGGFLGLSNIKFRPGSVV

SEQ ID NO: 52 - protein, 39 aa, synthetic construct
  REGION 1..39: note = polypeptide that can be used to activate T cells
STDYYQELQRDISEMFLFIYKQGGFLGLSNIKFRPGSVV

SEQ ID NO: 53 - protein, 39 aa, synthetic construct
  REGION 1..39: note = polypeptide that can be used to activate T cells
STDYYQELQRDISEMFLQIYKQGMFLGLSNIKFRPGSVV

SEQ ID NO: 54 - protein, 39 aa, synthetic construct
  REGION 1..39: note = polypeptide that can be used to activate T cells
STEYAQELQRDISEMFLQIYKQGGFLGLSNIKFRPGSVV

SEQ ID NO: 55 - protein, 27 aa, synthetic construct
  REGION 1..27: note = polypeptide that can be used to activate T cells
TQFNQYKTEAASRYNLTISDVSVSDVP

SEQ ID NO: 56 - protein, 32 aa, synthetic construct
  REGION 1..32: note = polypeptide that can be used to activate T cells
DVATQMNQYKTEAASNYNLTISDVSVSDVPFP

SEQ ID NO: 57 - protein, 29 aa, synthetic construct
  REGION 1..29: note = polypeptide that can be used to activate T cells
DVETQFNQYKTEAASRYNLTISDVSVSDV

SEQ ID NO: 58 - protein, 32 aa, synthetic construct
  REGION 1..32: note = polypeptide that can be used to activate T cells
DVETQFNQYKTEAASRYNLTISDVSVSDAPFP

SEQ ID NO: 59 - protein, 32 aa, synthetic construct
  REGION 1..32: note = polypeptide that can be used to activate T cells
DVETQFNQYKTEAASRYNLTISDLSPSDLPFP

SEQ ID NO: 60 - protein, 32 aa, synthetic construct
  REGION 1..32: note = polypeptide that can be used to activate T cells
DVATQFNQYKTEAPSRYNLTISDVSLSDVPFP

SEQ ID NO: 61 - protein, 32 aa, synthetic construct
  REGION 1..32: note = polypeptide that can be used to activate T cells
DVETQMNQYKTEAASRYNLTISDVSVSDVPFP

SEQ ID NO: 62 - protein, 32 aa, synthetic construct
  REGION 1..32: note = polypeptide that can be used to activate T cells
DVYTQFNQYKTEAASRYNLSISDVSVSDVPFP

SEQ ID NO: 63 - protein, 32 aa, synthetic construct; identical to SEQ ID NO: 3
  REGION 1..32: note = polypeptide that can be used to activate T cells

SEQ ID NO: 64 - protein, 33 aa, synthetic construct
  REGION 1..33: note = polypeptide that can be used to activate T cells
DVETQFNQYKTAAASRYNLVTISDVSVSDVPFP

SEQ ID NO: 65 - protein, 32 aa, synthetic construct
  REGION 1..32: note = polypeptide that can be used to activate T cells
LPAMEEGATILVTTKTNDYCKSLPAALSATEI

SEQ ID NO: 66 - protein, 32 aa, synthetic construct
  REGION 1..32: note = polypeptide that can be used to activate T cells
FPLPAMEEGATILVTTKTNDYCKSLPAALSAT

SEQ ID NO: 67 - protein, 37 aa, synthetic construct
  REGION 1..37: note = polypeptide that can be used to activate T cells
YPLPAMEAGATILVTTKTNDMCKSLPAALSATEIEKS

SEQ ID NO: 68 - protein, 37 aa, synthetic construct
  REGION 1..37: note = polypeptide that can be used to activate T cells
FPLPAMEEGATILVTTKTNDQCKSLPAALSATEIEKS

SEQ ID NO: 69 - protein, 37 aa, synthetic construct
  REGION 1..37: note = polypeptide that can be used to activate T cells
FPLPAMEEGATILVTTKTNDYYKSLPAALSATEIEKS

SEQ ID NO: 70 - protein, 35 aa, synthetic construct
  REGION 1..35: note = polypeptide that can be used to activate T cells
FPLPAMEEGATILVTTKTNDYCKSLPATSTATEIE

SEQ ID NO: 71 - protein, 34 aa, synthetic construct
  REGION 1..34: note = polypeptide that can be used to activate T cells
PAMEEGATILVTTKTNDYCKSLPAVLSATNIEKS

SEQ ID NO: 72 - protein, 37 aa, synthetic construct
  REGION 1..37: note = polypeptide that can be used to activate T cells
FPLPAMENGATIVVTTKTNDMCKSLPAALSATEIEKS

SEQ ID NO: 73 - protein, 37 aa, synthetic construct
  REGION 1..37: note = polypeptide that can be used to activate T cells
FPLPAMEEGATILVTTKTNDYCKSLPAALSATDIAKS

SEQ ID NO: 74 - protein, 37 aa, synthetic construct
  REGION 1..37: note = polypeptide that can be used to activate T cells
FPLPAMEPGATILVTTKTNYYCKSLPAALSATEIEKS

SEQ ID NO: 75 - protein, 22 aa, synthetic construct
  REGION 1..22: note = polypeptide that can be used to activate T cells
PDWRKDCSNNPVSVFWKTVSRR

SEQ ID NO: 76 - protein, 23 aa, synthetic construct
  REGION 1..23: note = polypeptide that can be used to activate T cells
YQSCPDWRKDCSNNPVSVFWKTV

SEQ ID NO: 77 - protein, 27 aa, synthetic construct
  REGION 1..27: note = polypeptide that can be used to activate T cells
YQSTCPDWRKDCSNNPVSVFWVTVSRR

SEQ ID NO: 78 - protein, 26 aa, synthetic construct
  REGION 1..26: note = polypeptide that can be used to activate T cells
YQSCPDWRKDQSNQPVSVMWKSVSKR

SEQ ID NO: 79 - protein, 26 aa, synthetic construct
  REGION 1..26: note = polypeptide that can be used to activate T cells
YQSCPDWRKDQSNNPVSVFWKTVSRR

SEQ ID NO: 80 - protein, 26 aa, synthetic construct
  REGION 1..26: note = polypeptide that can be used to activate T cells
YQSCPDWRKDNSNNPVSVFWKTVSRR

SEQ ID NO: 81 - protein, 26 aa, synthetic construct
  REGION 1..26: note = polypeptide that can be used to activate T cells
YQSCPDWRKDCSNNPVSVFFKTVSRR

SEQ ID NO: 82 - protein, 25 aa, synthetic construct
  REGION 1..25: note = polypeptide that can be used to activate T cells
YQSCPDWRKDYSNNPVSVFWKTVSR

SEQ ID NO: 83 - protein, 26 aa, synthetic construct
  REGION 1..26: note = polypeptide that can be used to activate T cells
YQSCPDTRKDCSNNPVSVFWKTVSRR

SEQ ID NO: 84 - protein, 26 aa, synthetic construct
  REGION 1..26: note = polypeptide that can be used to activate T cells
YQSQPDWRKDCSNNPVSVFWKSVSRR

SEQ ID NO: 85 - protein, 26 aa, synthetic construct
  REGION 1..26: note = polypeptide that can be used to activate T cells
ACDVVHVFLNGSRSKIFDKNSTFGSV

SEQ ID NO: 86 - protein, 22 aa, synthetic construct
  REGION 1..22: note = polypeptide that can be used to activate T cells
ACDVVHVMLNGSRSKIFDKNST

SEQ ID NO: 87 - protein, 26 aa, synthetic construct
  REGION 1..26: note = polypeptide that can be used to activate T cells
AFDVVHVMLNGSRSKIFDKNSTFGSE

SEQ ID NO: 88 - protein, 26 aa, synthetic construct
  REGION 1..26: note = polypeptide that can be used to activate T cells
ACDVIHVMLNGSRTKIFDKNSSFGSV

SEQ ID NO: 89 - protein, 26 aa, synthetic construct
  REGION 1..26: note = polypeptide that can be used to activate T cells
AQDVVHVMPNGTRSKLFDKNSTFGSV

SEQ ID NO: 90 - protein, 26 aa, synthetic construct
  REGION 1..26: note = polypeptide that can be used to activate T cells
APDVVHVMVNGSRSKIFDKNSTFGSV

SEQ ID NO: 91 - protein, 27 aa, synthetic construct
  REGION 1..27: note = polypeptide that can be used to activate T cells
ACDVVHVMLNGSRSPKIWDKNSTFGSV

SEQ ID NO: 92 - protein, 26 aa, synthetic construct
  REGION 1..26: note = polypeptide that can be used to activate T cells
ACDVVHVMLNGSRSKIQDKNSTFGTV

SEQ ID NO: 93 - protein, 26 aa, synthetic construct
  REGION 1..26: note = polypeptide that can be used to activate T cells
ACDVVHVMLNGSRSKIFDKNSTFFSV

SEQ ID NO: 94 - protein, 26 aa, synthetic construct
  REGION 1..26: note = polypeptide that can be used to activate T cells
ACDVVHNMLNGSRSKIFDKNSTFGSV

SEQ ID NO: 95 - protein, 38 aa, synthetic construct; identical to SEQ ID NO: 7
  REGION 1..38: note = polypeptide that can be used to activate T cells

SEQ ID NO: 96 - protein, 35 aa, synthetic construct
  REGION 1..35: note = polypeptide that can be used to activate T cells
DNGFGPQRSEVVSLFVTVPVSRPILTLRVPRAQAV

SEQ ID NO: 97 - protein, 35 aa, synthetic construct
  REGION 1..35: note = polypeptide that can be used to activate T cells
CTADNGFGPQRSEVVSLFVTVPVSRPILTLRVPRA

SEQ ID NO: 98 - protein, 37 aa, synthetic construct
  REGION 1..37: note = polypeptide that can be used to activate T cells
CTADNGFGPQRSEVVSLFVTVPVSRPILTLRVPRAQE

SEQ ID NO: 99 - protein, 38 aa, synthetic construct
  REGION 1..38: note = polypeptide that can be used to activate T cells
YTADNGFGPQRSEVVSLFFTVPVSRPILTLRVPRAQAV

SEQ ID NO: 100 - protein, 38 aa, synthetic construct
  REGION 1..38: note = polypeptide that can be used to activate T cells
WTADNGFGPQRSEVVSLFVTVPVSRPILTLRVPRNQAV

SEQ ID NO: 101 - protein, 38 aa, synthetic construct
  REGION 1..38: note = polypeptide that can be used to activate T cells
CTADNGFGPQRSNVVSLFVTVPVSRPILTLRVPRAQAV

SEQ ID NO: 102 - protein, 36 aa, synthetic construct
  REGION 1..36: note = polypeptide that can be used to activate T cells
TPDNFFGPQRSEVVSLFVTVPVSRPILTLRVPRAQA

SEQ ID NO: 103 - protein, 38 aa, synthetic construct
  REGION 1..38: note = polypeptide that can be used to activate T cells
CTADNGFGPQRSEVVSLFVTVPVSRPILTLRVPRNQAV

SEQ ID NO: 104 - protein, 37 aa, synthetic construct
  REGION 1..37: note = polypeptide that can be used to activate T cells
CTADNFFGPQRSEVVSLFVTVPVSRPILTLRVPRAQA

SEQ ID NO: 105 - protein, 27 aa, synthetic construct
  REGION 1..27: note = polypeptide that can be used to activate T cells
HSDTISLSVIVPVSRPILTFRAPRAQA

SEQ ID NO: 106 - protein, 28 aa, synthetic construct
  REGION 1..28: note = polypeptide that can be used to activate T cells
AQHSDTISLSVIVPVSRPILTFRAPRAQ

SEQ ID NO: 107 - protein, 27 aa, synthetic construct
  REGION 1..27: note = polypeptide that can be used to activate T cells
AQHSDTISLSVNVPVSRPILTFRAPRE

SEQ ID NO: 108 - protein, 29 aa, synthetic construct
  REGION 1..29: note = polypeptide that can be used to activate T cells
AQHSDTISLSVPVPVSRPILTFRAPRAQA

SEQ ID NO: 109 - protein, 29 aa, synthetic construct
  REGION 1..29: note = polypeptide that can be used to activate T cells
ADHSDTISLSVNVPVSRPILTFRAPRAQA

SEQ ID NO: 110 - protein, 29 aa, synthetic construct
  REGION 1..29: note = polypeptide that can be used to activate T cells
AFHSDTISLSVLVPVSRPILTFRAPRAQA

SEQ ID NO: 111 - protein, 26 aa, synthetic construct
  REGION 1..26: note = polypeptide that can be used to activate T cells
AQHSDTISLSVIVPVSRPWLTFRAPR

SEQ ID NO: 112 - protein, 29 aa, synthetic construct
  REGION 1..29: note = polypeptide that can be used to activate T cells
AQHSDTISLSVIVPVSRPILTFRAPRAQA

SEQ ID NO: 113 - protein, 29 aa, synthetic construct
  REGION 1..29: note = polypeptide that can be used to activate T cells
AQHSDTISLSVIVPVSRPIFFFRAPRAQA

SEQ ID NO: 114 - protein, 30 aa, synthetic construct
  REGION 1..30: note = polypeptide that can be used to activate T cells
AQHSDTISLSVIWVPVSRPILTFRAPRAQE

SEQ ID NO: 115 - protein, 25 aa, synthetic construct
  REGION 1..25: note = polypeptide that can be used to activate T cells
PDVTATPARLLFFAPTRRAAPLEEM

SEQ ID NO: 116 - protein, 27 aa, synthetic construct
  REGION 1..27: note = polypeptide that can be used to activate T cells
GAEVPDVTATPARLLFFAPTRRAAPLE

SEQ ID NO: 117 - protein, 29 aa, synthetic construct
  REGION 1..29: note = polypeptide that can be used to activate T cells
GAEVPDVTATPARLLFFAPTRRAAPLNEM

SEQ ID NO: 118 - protein, 29 aa, synthetic construct
  REGION 1..29: note = polypeptide that can be used to activate T cells
GAEVPDNTATPARLLFFPDTRRAAPLEEM

SEQ ID NO: 119 - protein, 28 aa, synthetic construct
  REGION 1..28: note = polypeptide that can be used to activate T cells
GAEVPVTATPARLLFMAPTRLANPLDEM

SEQ ID NO: 120 - protein, 29 aa, synthetic construct
  REGION 1..29: note = polypeptide that can be used to activate T cells
GATPARVTATPARLLFFAPTRRAAPLEEM

SEQ ID NO: 121 - protein, 27 aa, synthetic construct
  REGION 1..27: note = polypeptide that can be used to activate T cells
GAEVPDVTATPARLLFFAPTWRPPLEE

SEQ ID NO: 122 - protein, 29 aa, synthetic construct
  REGION 1..29: note = polypeptide that can be used to activate T cells
GAEVPDNTATPARLLFFAPTRRAAPLEEM

SEQ ID NO: 123 - protein, 27 aa, synthetic construct
  REGION 1..27: note = polypeptide that can be used to activate T cells
GAEVPDVTATPARLLFFAPTRRAAPLA

SEQ ID NO: 124 - protein, 29 aa, synthetic construct
  REGION 1..29: note = polypeptide that can be used to activate T cells
GAEVPDVTATPARLLFFAPTILAAPLEEM

SEQ ID NO: 125 - protein, 31 aa, synthetic construct
  REGION 1..31: note = polypeptide that can be used to activate T cells
RLNAALREKTSLSANNATLEKQLIELTRTNE

SEQ ID NO: 126 - protein, 36 aa, synthetic construct
  REGION 1..36: note = polypeptide that can be used to activate T cells
RLNAALREKTSLSANNATLNKQLIELTRTNELLKSK

SEQ ID NO: 127 - protein, 36 aa, synthetic construct
  REGION 1..36: note = polypeptide that can be used to activate T cells
RLNPPLREKTSLSANNATLEKQLIELTRTNELLKSK

SEQ ID NO: 128 - protein, 36 aa, synthetic construct
  REGION 1..36: note = polypeptide that can be used to activate T cells
RLNPPLNWKTSLSANNWTLEHQLIELTRTNELLKSK

SEQ ID NO: 129 - protein, 31 aa, synthetic construct
  REGION 1..31: note = polypeptide that can be used to activate T cells
LREKTSLSANNATLEKQLIELTRTNELLKSK

SEQ ID NO: 130 - protein, 36 aa, synthetic construct; identical to SEQ ID NO: 10
  REGION 1..36: note = polypeptide that can be used to activate T cells

SEQ ID NO: 131 - protein, 36 aa, synthetic construct
  REGION 1..36: note = polypeptide that can be used to activate T cells
RLNAALREKPPLSANNATLEKQLIELTRTNELLKSK

SEQ ID NO: 132 - protein, 36 aa, synthetic construct
  REGION 1..36: note = polypeptide that can be used to activate T cells
RLNAALFVKTSLSANNATLEKQLIELTRTNELLKSK

SEQ ID NO: 133 - protein, 36 aa, synthetic construct
  REGION 1..36: note = polypeptide that can be used to activate T cells
RLNAALREKTSLSANNATLEKQQKNLTRTNELLKSK

SEQ ID NO: 134 - protein, 34 aa, synthetic construct
  REGION 1..34: note = polypeptide that can be used to activate T cells
NNALREKTSLSANNATLEKQLIYLTRTNELLKSK

SEQ ID NO: 135 - protein, 36 aa, synthetic construct
  REGION 1..36: note = polypeptide that can be used to activate T cells
NGNQKNLRILSLELMKLRNKRETKMRGMMAKQEGME

SEQ ID NO: 136 - protein, 38 aa, synthetic construct
  REGION 1..38: note = polypeptide that can be used to activate T cells
SENGNQKNLRILSLNNMKLRNKRETKMRGMMAKQEGME

SEQ ID NO: 137 - protein, 38 aa, synthetic construct
  REGION 1..38: note = polypeptide that can be used to activate T cells
SENGNQKNLRILSLELMKLRNKRETKMRGPPAKQEGME

SEQ ID NO: 138 - protein, 38 aa, synthetic construct
  REGION 1..38: note = polypeptide that can be used to activate T cells
SENGNQKNLRILSLELHKLRNKQETKMRGMMAKQEGME

SEQ ID NO: 139 - protein, 38 aa, synthetic construct
  REGION 1..38: note = polypeptide that can be used to activate T cells
SENGNQKNLRILSLELMKLRNKRETKMRGMMANQEGME

SEQ ID NO: 140 - protein, 37 aa, synthetic construct
  REGION 1..37: note = polypeptide that can be used to activate T cells
SENGNQKNLYILSLELPFLRNKRETKMRGMMAKQEGM

SEQ ID NO: 141 - protein, 38 aa, synthetic construct
  REGION 1..38: note = polypeptide that can be used to activate T cells
SENGNQKNLRILSLEKQKLRNKRETKYRGMMAKQEGME

SEQ ID NO: 142 - protein, 38 aa, synthetic construct
  REGION 1..38: note = polypeptide that can be used to activate T cells
SKNGNQKNLRILSLELMKLRNKRETKMRGMMAKQEGME

SEQ ID NO: 143 - protein, 38 aa, synthetic construct
  REGION 1..38: note = polypeptide that can be used to activate T cells
SENGNQKNLRILSLELMKLINKRETKMRGMMAKQEGME

SEQ ID NO: 144 - protein, 38 aa, synthetic construct
  REGION 1..38: note = polypeptide that can be used to activate T cells
SENGNQKNLRILSLNLMKLRNKRETKMRGMMAKQEGME

SEQ ID NO: 145 - protein, 32 aa, synthetic construct
  REGION 1..32: note = polypeptide that can be used to activate T cells
AHLQATLLLQEKYDSMVQSLEDVTAQFESYKA

SEQ ID NO: 146 - protein, 29 aa, synthetic construct
  REGION 1..29: note = polypeptide that can be used to activate T cells
AHTQATLLLQEKYDSMVQSLEDVTAQFES

SEQ ID NO: 147 - protein, 28 aa, synthetic construct
  REGION 1..28: note = polypeptide that can be used to activate T cells
ATLLLQEKYDSMVQSLEDVTAQFESYKA

SEQ ID NO: 148 - protein, 32 aa, synthetic construct
  REGION 1..32: note = polypeptide that can be used to activate T cells
AHTQATPPLQNKYDSMVQSLEDVTAQFESYKA

SEQ ID NO: 149 - protein, 32 aa, synthetic construct
  REGION 1..32: note = polypeptide that can be used to activate T cells
AHTQATLLLQYKYDSMVQSLWDVTAQFFSYKA

SEQ ID NO: 150 - protein, 33 aa, synthetic construct
  REGION 1..33: note = polypeptide that can be used to activate T cells
AHTQATLLLQEKYYSMIVQSLVDVTAQFESYNA

SEQ ID NO: 151 - protein, 32 aa, synthetic construct
  REGION 1..32: note = polypeptide that can be used to activate T cells
AHTQATLLLQYKYPSMVQSLEDVTAQFESYKA

SEQ ID NO: 152 - protein, 32 aa, synthetic construct
  REGION 1..32: note = polypeptide that can be used to activate T cells
AHTQKNLLLQNKYDSMVQSLPDVTAQFESYKA

SEQ ID NO: 153 - protein, 32 aa, synthetic construct
  REGION 1..32: note = polypeptide that can be used to activate T cells
AHTQATLLLQYKYDSMVQSLNFVTAQFFSYKA

SEQ ID NO: 154 - protein, 32 aa, synthetic construct
  REGION 1..32: note = polypeptide that can be used to activate T cells
AHTQATLLLQFFYDSMVQSLEDVTAQFESYKA

SEQ ID NO: 155 - protein, 37 aa, synthetic construct
  REGION 1..37: note = polypeptide that can be used to activate T cells
YTWKALGQAANESHNGSILPISWRWGESDMTFICVAR

SEQ ID NO: 156 - protein, 36 aa, synthetic construct
  REGION 1..36: note = polypeptide that can be used to activate T cells
DVIYTWKALGQAANESHNGSILPISWRWGESDMTFI

SEQ ID NO: 157 - protein, 36 aa, synthetic construct
  REGION 1..36: note = polypeptide that can be used to activate T cells
DVIYTWKALGQAANESHNGSILPISWRWGESDMTFA

SEQ ID NO: 158 - protein, 40 aa, synthetic construct
  REGION 1..40: note = polypeptide that can be used to activate T cells
DVIYTWKNLGQAANESHNGSILPISWRWGESDMTFICVAR

SEQ ID NO: 159 - protein, 40 aa, synthetic construct
  REGION 1..40: note = polypeptide that can be used to activate T cells
DVIYTWKALGQYYNESHNGSILPISWRWGESDMTFICVAR

SEQ ID NO: 160 - protein, 40 aa, synthetic construct
  REGION 1..40: note = polypeptide that can be used to activate T cells
DVIYTWYYLGQWYNESHNGSILPISWNWGESDMTFICVAR

SEQ ID NO: 161 - protein, 40 aa, synthetic construct
  REGION 1..40: note = polypeptide that can be used to activate T cells
DVIYTWQYLGQAANESHNGSILPISWRWGESDMTFICVAR

SEQ ID NO: 162 - protein, 36 aa, synthetic construct
  REGION 1..36: note = polypeptide that can be used to activate T cells
DVNYTWKALGQVINESHNGSILPISWRWGESDMTFI

SEQ ID NO: 163 - protein, 41 aa, synthetic construct
  REGION 1..41: note = polypeptide that can be used to activate T cells
DVIYYWQENLGQAANESHNGSILPISWRWGESDMTFICVAR

SEQ ID NO: 164 - protein, 40 aa, synthetic construct
  REGION 1..40: note = polypeptide that can be used to activate T cells
DVIYTWKALGQAANESHNGSVVPISWRWGESDMTFICVAR

SEQ ID NO: 165 - protein, 24 aa, synthetic construct
  REGION 1..24: note = polypeptide that can be used to activate T cells
LGLFLWFLKRERQEENNPKGRSSK

SEQ ID NO: 166 - protein, 24 aa, synthetic construct
  REGION 1..24: note = polypeptide that can be used to activate T cells
LWFLKRERQEENNPKGRSSKYGLL

SEQ ID NO: 167 - protein, 28 aa, synthetic construct
  REGION 1..28: note = polypeptide that can be used to activate T cells
LGLFLWFLKRERQEENNPKGRSSKYGLL

SEQ ID NO: 168 - protein, 28 aa, synthetic construct
  REGION 1..28: note = polypeptide that can be used to activate T cells
LGLFLWFLKRERQVDSIVWGRSSKYGLL

SEQ ID NO: 169 - protein, 28 aa, synthetic construct
  REGION 1..28: note = polypeptide that can be used to activate T cells
LGLFLWFLKRERQPPNNPKGRSSKYGLL

SEQ ID NO: 170 - protein, 28 aa, synthetic construct
  REGION 1..28: note = polypeptide that can be used to activate T cells
LGLFLWFLKRERQYYNNPKGRSSKYGLL

SEQ ID NO: 171 - protein, 28 aa, synthetic construct
  REGION 1..28: note = polypeptide that can be used to activate T cells
LGLFLWFLPRERQEENNPNGRSSKYGLL

SEQ ID NO: 172 - protein, 28 aa, synthetic construct
  REGION 1..28: note = polypeptide that can be used to activate T cells
LGLFLWFLKRERQNINNPKGRSSKYNLL

SEQ ID NO: 173 - protein, 28 aa, synthetic construct
  REGION 1..28: note = polypeptide that can be used to activate T cells
LGLFLWFLKRERQEENNPKGRSSKYGLA

SEQ ID NO: 174 - protein, 28 aa, synthetic construct
  REGION 1..28: note = polypeptide that can be used to activate T cells
LGLFLWFLKRYYQEKNNPKGRSSKYGLL

SEQ ID NO: 175 - protein, 38 aa, synthetic construct; identical to SEQ ID NO: 15
  REGION 1..38: note = polypeptide that can be used to activate T cells

SEQ ID NO: 176 - protein, 25 aa, synthetic construct
  REGION 1..25: note = polypeptide that can be used to activate T cells
NSPDRVKRPMNAFMVWSRGQRRKMA

SEQ ID NO: 177 - protein, 36 aa, synthetic construct
  REGION 1..36: note = polypeptide that can be used to activate T cells
PDRVKRPMNAFMVWSRGQRRKMAQENPKMHNSEISK

SEQ ID NO: 178 - protein, 38 aa, synthetic construct
  REGION 1..38: note = polypeptide that can be used to activate T cells
NSPDRVKRPMNAFMVWSRGQPPKMAQENPKMHNSEISA

SEQ ID NO: 179 - protein, 37 aa, synthetic construct
  REGION 1..37: note = polypeptide that can be used to activate T cells
SPNRVKRPMNAFMVWSRGQRVKMAQENPKMHNSEISK

SEQ ID NO: 180 - protein, 38 aa, synthetic construct
  REGION 1..38: note = polypeptide that can be used to activate T cells
NSPDPVKRPMNFFMVWSLGQRRKMAQENPKMHNSEISK

SEQ ID NO: 181 - protein, 38 aa, synthetic construct
  REGION 1..38: note = polypeptide that can be used to activate T cells
NSPDRVKRPMNAFMVWSRGQRRKMAQNNPAMHNSEISK

SEQ ID NO: 182 - protein, 38 aa, synthetic construct
  REGION 1..38: note = polypeptide that can be used to activate T cells
NSPDRVKRPMNAFMVWSWGQRRKMAQYNPKMHNSEISE

SEQ ID NO: 183 - protein, 38 aa, synthetic construct
  REGION 1..38: note = polypeptide that can be used to activate T cells
NSPDRVYRPMNAFMVWSRGQRRKMAQENPKMHNSEISK

SEQ ID NO: 184 - protein, 35 aa, synthetic construct
  REGION 1..35: note = polypeptide that can be used to activate T cells
NSPDRVKRPMNAFMYFSRGQRRKMAQENPKMHNSE

SEQ ID NO: 185 - protein, 31 aa, synthetic construct
  REGION 1..31: note = polypeptide that can be used to activate T cells
TEKRPFIDEAKRLRALHMKEHPDYKYRPRRK

SEQ ID NO: 186 - protein, 35 aa, synthetic construct
  REGION 1..35: note = polypeptide that can be used to activate T cells
EWKLLSETEKRPFIDEAKRLRALHMKEHPDYKYRP

SEQ ID NO: 187 - protein, 38 aa, synthetic construct
  REGION 1..38: note = polypeptide that can be used to activate T cells
EWKLLSYTEKRPFIYEANRLRALHMKEHPDYKYRPRRK

SEQ ID NO: 188 - protein, 38 aa, synthetic construct
  REGION 1..38: note = polypeptide that can be used to activate T cells
EWKLLSETEKRPFIDEAKRLYPLHMKNHPDYKYRPRRK

SEQ ID NO: 189 - protein, 38 aa, synthetic construct
  REGION 1..38: note = polypeptide that can be used to activate T cells
EWKLLSWTQYRPFIDEAKRLRALHMKEHPDYKYRPRRK

SEQ ID NO: 190 - protein, 38 aa, synthetic construct
  REGION 1..38: note = polypeptide that can be used to activate T cells
EWKLLYWTNKRPFIDEAKRLRALHMKEHPDYKYRPRRA

SEQ ID NO: 191 - protein, 38 aa, synthetic construct
  REGION 1..38: note = polypeptide that can be used to activate T cells
EWKLLSETEKRPFIDVSALQRALHMKEHPDYKYRPRRK

SEQ ID NO: 192 - protein, 34 aa, synthetic construct
  REGION 1..34: note = polypeptide that can be used to activate T cells
LSETEKRPFIDEAKRLRALHMKEHPDYKYRPRRK

SEQ ID NO: 193 - protein, 38 aa, synthetic construct
  REGION 1..38: note = polypeptide that can be used to activate T cells
EWKLLSETEKRPFIDMAKRLNALHMKEHPDYYYRPRRA

SEQ ID NO: 194 - protein, 33 aa, synthetic construct
  REGION 1..33: note = polypeptide that can be used to activate T cells
EWKLLSETYKRPFIMEAKRLRNLHMKEHPDYKY

SEQ ID NO: 195 - protein, 27 aa, synthetic construct
  REGION 1..27: note = polypeptide that can be used to activate T cells
PNPADGTPKVYYPSGQPASAAGAPAGQ

SEQ ID NO: 196 - protein, 26 aa, synthetic construct
  REGION 1..26: note = polypeptide that can be used to activate T cells
AAAPNPADGTPKVLLLSGQPASAAGA

SEQ ID NO: 197 - protein, 30 aa, synthetic construct
  REGION 1..30: note = polypeptide that can be used to activate T cells
AAAPNPADGTPKVLLLSGQPASYYGAPAGQ

SEQ ID NO: 198 - protein, 30 aa, synthetic construct
  REGION 1..30: note = polypeptide that can be used to activate T cells
AAAPNPADGTPMVLLLSGQPASNNGAPAGQ

SEQ ID NO: 199 - protein, 28 aa, synthetic construct
  REGION 1..28: note = polypeptide that can be used to activate T cells
APNPATGTPKVLLLSGQPASAAGAPAGQ

SEQ ID NO: 200 - protein, 29 aa, synthetic construct
  REGION 1..29: note = polypeptide that can be used to activate T cells
AAPNPLYGTPKVLLLSGQPASAAGAPAGQ

SEQ ID NO: 201 - protein, 31 aa, synthetic construct
  REGION 1..31: note = polypeptide that can be used to activate T cells
AAAPNPADGTPKVLYLLSGQPASAAGAPAGQ

SEQ ID NO: 202 - protein, 30 aa, synthetic construct
  REGION 1..30: note = polypeptide that can be used to activate T cells
AAAPNPADGTPAVLLLSGQPASNNGAPAGQ

SEQ ID NO: 203 - protein, 31 aa, synthetic construct
  REGION 1..31: note = polypeptide that can be used to activate T cells
AAAPNPADGTPKVLLYLSGQPASAAGAPAGE

SEQ ID NO: 204 - protein, 30 aa, synthetic construct
  REGION 1..30: note = polypeptide that can be used to activate T cells
AAAPNPNDGTPKVLLLSGQPASNYGAPAGQ

SEQ ID NO: 205 - protein, 40 aa, synthetic construct
  REGION 1..40: note = polypeptide that can be used to activate T cells
SYSDILLGILDNLDPVMFFKCPSPEPASLEELPEVYPEGP

SEQ ID NO: 206 - protein, 35 aa, synthetic construct
  REGION 1..35: note = polypeptide that can be used to activate T cells
SDILLGILDNLDPVMFFKCPSPEPASLEELPEVYP

SEQ ID NO: 207 - protein, 36 aa, synthetic construct
  REGION 1..36: note = polypeptide that can be used to activate T cells
SDILLGILDNLDPVMFFQCPSPNPASLYYLPEVYPE

SEQ ID NO: 208 - protein, 38 aa, synthetic construct
  REGION 1..38: note = polypeptide that can be used to activate T cells
SDILLGILDNLDPVMFFKCPSPDPASLVILPEVYPEGE

SEQ ID NO: 209 - protein, 38 aa, synthetic construct
  REGION 1..38: note = polypeptide that can be used to activate T cells
SDILLGILDNLDPVMFFNQPSPEPASLEELPEVYPEGP

SEQ ID NO: 210 - protein, 38 aa, synthetic construct
  REGION 1..38: note = polypeptide that can be used to activate T cells
SDILLGILDNLDPVMFFKCPSPEPASLEELPEVYQELP

SEQ ID NO: 211 - protein, 38 aa, synthetic construct
  REGION 1..38: note = polypeptide that can be used to activate T cells
SDILLGILDNLDPVMFFQNPSPEPASLEELPEVYPEGP

SEQ ID NO: 212 - protein, 38 aa, synthetic construct
  REGION 1..38: note = polypeptide that can be used to activate T cells
SDILLGILDNLDPVMFFKNPSPAPASLEELPEVYPEGP

SEQ ID NO: 213 - protein, 38 aa, synthetic construct
  REGION 1..38: note = polypeptide that can be used to activate T cells
SDILLGILDNLDPVMFFFVPSPTPASLEELPEVYPEGE

SEQ ID NO: 214 - protein, 38 aa, synthetic construct
  REGION 1..38: note = polypeptide that can be used to activate T cells
SDILLGILDNELIRMFFKCPSPEPASLEELPEVYPEGP

SEQ ID NO: 215 - protein, 28 aa, synthetic construct
  REGION 1..28: note = polypeptide that can be used to activate T cells
PETVFKRPRECDSPSYQKRQRMALLARK

SEQ ID NO: 216 - protein, 28 aa, synthetic construct
  REGION 1..28: note = polypeptide that can be used to activate T cells
AVDPETVFKRPRECDSPSYQKRQRMALL

SEQ ID NO: 217 - protein, 29 aa, synthetic construct
  REGION 1..29: note = polypeptide that can be used to activate T cells
AVDPETVFKRPRYMDSPSYQKRQRMALLA

SEQ ID NO: 218 - protein, 30 aa, synthetic construct
  REGION 1..30: note = polypeptide that can be used to activate T cells
VDPATVFKRPYNWDSPSYQKRQRMALLARK

SEQ ID NO: 219 - protein, 31 aa, synthetic construct
  REGION 1..31: note = polypeptide that can be used to activate T cells
AVDPETVFKRPRRKDSPSYQKRQRMALLARK

SEQ ID NO: 220 - protein, 32 aa, synthetic construct
  REGION 1..32: note = polypeptide that can be used to activate T cells
AVDPEYTVFKRPRVCDSPSYQKRQRMALLARK

SEQ ID NO: 221 - protein, 31 aa, synthetic construct
  REGION 1..31: note = polypeptide that can be used to activate T cells
AVDPVTVFRKPRECDSPSYQKRQRMALLARK

SEQ ID NO: 222 - protein, 31 aa, synthetic construct
  REGION 1..31: note = polypeptide that can be used to activate T cells
AVDPETVFKRPRYIDSPSYQKRQRMALLARK

SEQ ID NO: 223 - protein, 31 aa, synthetic construct
  REGION 1..31: note = polypeptide that can be used to activate T cells
AVDPPTVFFRPREYYSPSYQKRQRMALLARK

SEQ ID NO: 224 - protein, 31 aa, synthetic construct
  REGION 1..31: note = polypeptide that can be used to activate T cells
AVDPQTVFFRPREWDSPSYQKRQRMALLARK

SEQ ID NO: 225 - protein, 31 aa, synthetic construct
  REGION 1..31: note = polypeptide that can be used to activate T cells
VQGPTAVRKRFFESIIKEAARCMRRDFVKHL

SEQ ID NO: 226 - protein, 29 aa, synthetic construct
  REGION 1..29: note = polypeptide that can be used to activate T cells
LEGVQGPTAVRKRFFESIIKEAARCMRRD

SEQ ID NO: 227 - protein, 34 aa, synthetic construct
  REGION 1..34: note = polypeptide that can be used to activate T cells
LEGVQYPTAVRKRFFESIIKENFRCMRRDFVKHL

SEQ ID NO: 228 - protein, 32 aa, synthetic construct
  REGION 1..32: note = polypeptide that can be used to activate T cells
LEGVQGPTAVRKRFFESILTNRCMRRDFVKHL

SEQ ID NO: 229 - protein, 34 aa, synthetic construct
  REGION 1..34: note = polypeptide that can be used to activate T cells
LEGVQGPTAVRKRFFESIIKEPVRCMRRDFVKHL

SEQ ID NO: 230 - protein, 34 aa, synthetic construct
  REGION 1..34: note = polypeptide that can be used to activate T cells
LLGVQNPTAVRKRFFESIIKRKARCMRRDFVKHL

SEQ ID NO: 231 - protein, 34 aa, synthetic construct
  REGION 1..34: note = polypeptide that can be used to activate T cells
LEGVQGPTAVRKRFFESIIKEFFRCMRRDFVKHL

SEQ ID NO: 232 - protein, 34 aa, synthetic construct
  REGION 1..34: note = polypeptide that can be used to activate T cells
LEGWQGPTAVRKRFFESIINYPARCMRRDFVKHL

SEQ ID NO: 233 - protein, 34 aa, synthetic construct
  REGION 1..34: note = polypeptide that can be used to activate T cells
LEGVQGPTAVRKRFFESIIKEAARNMRRDFVKHL

SEQ ID NO: 234 - protein, 33 aa, synthetic construct
  REGION 1..33: note = polypeptide that can be used to activate T cells
LEGVQGPTAVRKRFFESIIKEAARQMRRDFVKH

SEQ ID NO: 235 - protein, 25 aa, synthetic construct
  REGION 1..25: note = polypeptide that can be used to activate T cells
TFPDLESEFQAALSRKVAELVHFLL

SEQ ID NO: 236 - protein, 26 aa, synthetic construct
  REGION 1..26: note = polypeptide that can be used to activate T cells
FPDLESEFQAALSRKVAELVHFLLLK

SEQ ID NO: 237 - protein, 27 aa, synthetic construct
  REGION 1..27: note = polypeptide that can be used to activate T cells
TFPDLESEFQAALSRKVAELVHFLLLK

SEQ ID NO: 238 - protein, 27 aa, synthetic construct
  REGION 1..27: note = polypeptide that can be used to activate T cells
TFPDLESEFQNYLSRKVAELVHFLLLK

SEQ ID NO: 239 - protein, 27 aa, synthetic construct
  REGION 1..27: note = polypeptide that can be used to activate T cells
TFPDLESEFQYYLSRKVANLVHFLLLK

SEQ ID NO: 240 - protein, 26 aa, synthetic construct
  REGION 1..26: note = polypeptide that can be used to activate T cells
TFPDLESEFQAPLSRKVAELVHFLLL

SEQ ID NO: 241 - protein, 27 aa, synthetic construct
  REGION 1..27: note = polypeptide that can be used to activate T cells
TFPDLESEFQAAVRRKVAELVHFLFLE

SEQ ID NO: 242 - protein, 27 aa, synthetic construct
  REGION 1..27: note = polypeptide that can be used to activate T cells
TFPDLESEFQSSLSRKVAELVHFLLLK

SEQ ID NO: 243 - protein, 26 aa, synthetic construct
  REGION 1..26: note = polypeptide that can be used to activate T cells
TFPDLESEFQAALSYYVAELVHFLLL

SEQ ID NO: 244 - protein, 27 aa, synthetic construct
  REGION 1..27: note = polypeptide that can be used to activate T cells
TFPDLESEFQANLSRNVAELVHFLLLK

SEQ ID NO: 245 - protein, 27 aa, synthetic construct
  REGION 1..27: note = polypeptide that can be used to activate T cells
SLFREALSNKVDELAHFLLRKYRAKEL

SEQ ID NO: 246 - protein, 28 aa, synthetic construct
  REGION 1..28: note = polypeptide that can be used to activate T cells
PDAESLFREALSNKVDELAHFLLRKYRA

SEQ ID NO: 247 - protein, 31 aa, synthetic construct
  REGION 1..31: note = polypeptide that can be used to activate T cells
PDAESYFREALSNKVDSLAHFLLRKYRAKEL

SEQ ID NO: 248 - protein, 31 aa, synthetic construct
  REGION 1..31: note = polypeptide that can be used to activate T cells
PDAESLFREALSNQVDELAHFLLRSYRAKEL

SEQ ID NO: 249 - protein, 31 aa, synthetic construct
  REGION 1..31: note = polypeptide that can be used to activate T cells
PDAESLMREALSNKVDELAHFQLRKYRAKEL

SEQ ID NO: 250 - protein, 31 aa, synthetic construct
  REGION 1..31: note = polypeptide that can be used to activate T cells
PDAESLFRQALSNKVDELAHIQLRKYRAKEL

SEQ ID NO: 251 - protein, 29 aa, synthetic construct
  REGION 1..29: note = polypeptide that can be used to activate T cells
PDAESLFREALSNKVDELAHFLLRKYRAK

SEQ ID NO: 252 - protein, 31 aa, synthetic construct
  REGION 1..31: note = polypeptide that can be used to activate T cells
VDAESLFREALSNKVDELAHFLLRKYRAKEL

SEQ ID NO: 253 - protein, 30 aa, synthetic construct
  REGION 1..30: note = polypeptide that can be used to activate T cells
PDAESLFRENLSNKVDELAHFLLRKYRAKE

SEQ ID NO: 254 - protein, 31 aa, synthetic construct
  REGION 1..31: note = polypeptide that can be used to activate T cells
PDAESLFREALSNKVDELAHFLLNKYRAKEL

SEQ ID NO: 255 - protein, 25 aa, synthetic construct
  REGION 1..25: note = polypeptide that can be used to activate T cells
PDLESEFQAALSRKVAKLVHFLLLK

SEQ ID NO: 256 - protein, 24 aa, synthetic construct
  REGION 1..24: note = polypeptide that can be used to activate T cells
TFPDLESEFQAALSRKVAKLVHFL

SEQ ID NO: 257 - protein, 27 aa, synthetic construct
  REGION 1..27: note = polypeptide that can be used to activate T cells
TFPDLESEFQNYLSRKVAKLVHFLLLK

SEQ ID NO: 258 - protein, 27 aa, synthetic construct
  REGION 1..27: note = polypeptide that can be used to activate T cells
TFPDLESEFQANLSRKVAKLVHFLLLE

SEQ ID NO: 259 - protein, 27 aa, synthetic construct
  REGION 1..27: note = polypeptide that can be used to activate T cells
TFPDLESEFQAALSRKVAKLVHFLLLK

SEQ ID NO: 260 - protein, 27 aa, synthetic construct
  REGION 1..27: note = polypeptide that can be used to activate T cells
TFPDLESEFQAALSRKVAKLVHFLYLL

SEQ ID NO: 261 - protein, 26 aa, synthetic construct
  REGION 1..26: note = polypeptide that can be used to activate T cells
FPDLNSEFQAALSRNVAKLVHFLLLK

SEQ ID NO: 262 - protein, 26 aa, synthetic construct
  REGION 1..26: note = polypeptide that can be used to activate T cells
TFPDLESEFQAALSRKVWKLVHFLLA

SEQ ID NO: 263 - protein, 27 aa, synthetic construct
  REGION 1..27: note = polypeptide that can be used to activate T cells
SFPDLESEFQAALSRKVNKLVHFLYLK

SEQ ID NO: 264 - protein, 27 aa, synthetic construct
  REGION 1..27: note = polypeptide that can be used to activate T cells
TFPDLESEFQAALSRKVYKLVHFLLLK

SEQ ID NO: 265 - protein, 36 aa, synthetic construct
  REGION 1..36: note = polypeptide that can be used to activate T cells
YLEYRQVPGSDPACYEFLWGPRALIETSYVKVLHHM

SEQ ID NO: 266 - protein, 41 aa, synthetic construct
  REGION 1..41: note = polypeptide that can be used to activate T cells
FVQENYLEYRQHFGSDPACYEFLWGPRALIETSYVKVLHHM

SEQ ID NO: 267 - protein, 41 aa, synthetic construct
  REGION 1..41: note = polypeptide that can be used to activate T cells
FVQENYLEMWQVPGSDPACYEFLWGPRALIETSYVKVLHHM

SEQ ID NO: 268 - protein, 41 aa, synthetic construct
  REGION 1..41: note = polypeptide that can be used to activate T cells
FVQENYLEYRQAALSDPACYEFLWGPRALIETSYVKVLHHM

SEQ ID NO: 269 - protein, 41 aa, synthetic construct
  REGION 1..41: note = polypeptide that can be used to activate T cells
FVQENYLEYRQVPGSDPACYEFLWGPRALIETSYVHFLHHM

SEQ ID NO: 270 - protein, 41 aa, synthetic construct; identical to SEQ ID NO: 24
  REGION 1..41: note = polypeptide that can be used to activate T cells

SEQ ID NO: 271 - protein, 41 aa, synthetic construct
  REGION 1..41: note = polypeptide that can be used to activate T cells
FVQENYLEYRQVPGSDPAWYEFLWGPRALIETSYVKVLHHM

SEQ ID NO: 272 - protein, 41 aa, synthetic construct
  REGION 1..41: note = polypeptide that can be used to activate T cells
FVQENYLEYRQVPGSDPAYYEFLWGPRALIETSYVKVLHHM

SEQ ID NO: 273 - protein, 41 aa, synthetic construct
  REGION 1..41: note = polypeptide that can be used to activate T cells
FVQENYLEYRQVPGSDPACYEFFWGQRALIETSYVKVLHHM

SEQ ID NO: 274 - protein, 41 aa, synthetic construct
  REGION 1..41: note = polypeptide that can be used to activate T cells
FVQENYLEYRQVPGSDPACYIFMWGPRALIETSYVKVLHHE

SEQ ID NO: 275 - protein, 27 aa, synthetic construct
  REGION 1..27: note = polypeptide that can be used to activate T cells
PLQRPVSSFFSYTLASLLQSSHESPQS

SEQ ID NO: 276 - protein, 26 aa, synthetic construct
  REGION 1..26: note = polypeptide that can be used to activate T cells
LQRPVSSFFSYTLASLLQSSHESPQS

SEQ ID NO: 277 - protein, 25 aa, synthetic construct
  REGION 1..25: note = polypeptide that can be used to activate T cells
QRPVSSFFSYTLASLLQSSHESPQS

SEQ ID NO: 278 - protein, 27 aa, synthetic construct
  REGION 1..27: note = polypeptide that can be used to activate T cells
SPLQRPVSSFFSYTLASLLQSSHESPQ

SEQ ID NO: 279 - protein, 28 aa, synthetic construct
  REGION 1..28: note = polypeptide that can be used to activate T cells
SPLQRPVSTFFSYTLASLLQSSHESPQQ

SEQ ID NO: 280 - protein, 28 aa, synthetic construct
  REGION 1..28: note = polypeptide that can be used to activate T cells
SPLQRPVSSFFSYTLASLLQSSHESPQN

SEQ ID NO: 281 - protein, 28 aa, synthetic construct
  REGION 1..28: note = polypeptide that can be used to activate T cells
SPLQRPVWSFFSYTLASLLQSSHESPQA

SEQ ID NO: 282 - protein, 28 aa, synthetic construct
  REGION 1..28: note = polypeptide that can be used to activate T cells
QPLQRPVSSFFSYTLASLIQSSHESPQS

SEQ ID NO: 283 - protein, 28 aa, synthetic construct
  REGION 1..28: note = polypeptide that can be used to activate T cells
NPLQRPVSSFFSYTLASLLQSSHESPQS

SEQ ID NO: 284 - protein, 28 aa, synthetic construct
  REGION 1..28: note = polypeptide that can be used to activate T cells
APLQRPVSSFFSYTLASLLQSSHESPQS

SEQ ID NO: 285 - protein, 24 aa, synthetic construct
  REGION 1..24: note = polypeptide that can be used to activate T cells
SSTSSSLSKSSPESPLQSPVISFS

SEQ ID NO: 286 - protein, 21 aa, synthetic construct
  REGION 1..21: note = polypeptide that can be used to activate T cells
SSLSKSSPESPLQSPVISFSA

SEQ ID NO: 287 - protein, 25 aa, synthetic construct
  REGION 1..25: note = polypeptide that can be used to activate T cells
SSTSASLSKSSPESPLQSPVISFSN

SEQ ID NO: 288 - protein, 22 aa, synthetic construct
  REGION 1..22: note = polypeptide that can be used to activate T cells
SSTSSSLSKNSPESPLQSPVIS

SEQ ID NO: 289 - protein, 25 aa, synthetic construct
  REGION 1..25: note = polypeptide that can be used to activate T cells
SSTSSSLSKSNPESPLQSPVISFSS

SEQ ID NO: 290 - protein, 25 aa, synthetic construct
  REGION 1..25: note = polypeptide that can be used to activate T cells
SSTSYYLSKSSIESPLQSPVISFSS

SEQ ID NO: 291 - protein, 25 aa, synthetic construct
  REGION 1..25: note = polypeptide that can be used to activate T cells
SSTSSSLSKSSVESPLQSPVISFSS

SEQ ID NO: 292 - protein, 25 aa, synthetic construct
  REGION 1..25: note = polypeptide that can be used to activate T cells
SSTSWSLSKTSPQSPLQSPVISFSE

SEQ ID NO: 293 - protein, 25 aa, synthetic construct
  REGION 1..25: note = polypeptide that can be used to activate T cells
SSTSMSLSKSSPNSPLQSPVISFSA

SEQ ID NO: 294 - protein, 25 aa, synthetic construct
  REGION 1..25: note = polypeptide that can be used to activate T cells
SSTSSSLSKSSPENPLQSPVISFSS

SEQ ID NO: 295 - protein, 33 aa, synthetic construct
  REGION 1..33: note = polypeptide that can be used to activate T cells
PRYEFLWGPRAHSEVIKRKVVEFLAMLKNTVPI

SEQ ID NO: 296 - protein, 37 aa, synthetic construct
  REGION 1..37: note = polypeptide that can be used to activate T cells
NSSPPRYQFLWGVRAHSEVIKRKVVEFLAMLKNTVPI

SEQ ID NO: 297 - protein, 31 aa, synthetic construct
  REGION 1..31: note = polypeptide that can be used to activate T cells
NSSPPRYEFLWGPRAHSEVIKRKVVEFLAML

SEQ ID NO: 298 - protein, 37 aa, synthetic construct
  REGION 1..37: note = polypeptide that can be used to activate T cells
NSTPPRYEFLWNPRAHSEVIIRKVVEFLAMLKNTVPI

SEQ ID NO: 299 - protein, 35 aa, synthetic construct
  REGION 1..35: note = polypeptide that can be used to activate T cells
SPPRYEFLWGPNAHSEVIKRKVVEFLAMLKNTVPI

SEQ ID NO: 300 - protein, 37 aa, synthetic construct
  REGION 1..37: note = polypeptide that can be used to activate T cells
NSSPPRYNFLWGPRAHSIVIARKVVEFLAMLRNTVPI

SEQ ID NO: 301 - protein, 37 aa, synthetic construct
  REGION 1..37: note = polypeptide that can be used to activate T cells
NSSPPRYEFLWGPRNHSEVIKRKVVEFLVMLKNTVPI

SEQ ID NO: 302 - protein, 32 aa, synthetic construct
  REGION 1..32: note = polypeptide that can be used to activate T cells
RYEFLWGPRAHSEVIKRKVVEFLAMLKNTVPI

SEQ ID NO: 303 - protein, 37 aa, synthetic construct; identical to SEQ ID NO: 27
  REGION 1..37: note = polypeptide that can be used to activate T cells

SEQ ID NO: 304 - protein, 34 aa, synthetic construct
  REGION 1..34: note = polypeptide that can be used to activate T cells
SSPPRYEFLWGPRAHSEVIKRKVVEFLAMLKNTL

SEQ ID NO: 305 - protein, 31 aa, synthetic construct
  REGION 1..31: note = polypeptide that can be used to activate T cells
ARGPESRLLEFYLAMPFATPMEAELARRSLA

SEQ ID NO: 306 - protein, 35 aa, synthetic construct
  REGION 1..35: note = polypeptide that can be used to activate T cells
GPESRLLEFYLIMPFATPMEAELARRSLAQDAPPL

SEQ ID NO: 307 - protein, 37 aa, synthetic construct
  REGION 1..37: note = polypeptide that can be used to activate T cells
ARGPESRLLKFYLVMPFATPMEAELARRSLAQDAPPL

SEQ ID NO: 308 - protein, 37 aa, synthetic construct
  REGION 1..37: note = polypeptide that can be used to activate T cells
ARGPESRLLKFYLTMPFATPMEAELARDSLAQDAPPL

SEQ ID NO: 309 - protein, 34 aa, synthetic construct
  REGION 1..34: note = polypeptide that can be used to activate T cells
ARGPESRLLEFYLAMPFATPMEAELARRSLAQDA

SEQ ID NO: 310 - protein, 37 aa, synthetic construct
  REGION 1..37: note = polypeptide that can be used to activate T cells
ARGPESRLLEFYLAMPFATPDTRPLARRSLAQDAPPL

SEQ ID NO: 311 - protein, 37 aa, synthetic construct
  REGION 1..37: note = polypeptide that can be used to activate T cells
ARGPESRLLKFYLAMPFATPMEAELARRSLAQDAPPL

SEQ ID NO: 312 - protein, 37 aa, synthetic construct
  REGION 1..37: note = polypeptide that can be used to activate T cells
ARGPNSRLLIFYLYCPFATPMRARLSRRTLAQDAPPL

SEQ ID NO: 313 - protein, 37 aa, synthetic construct
  REGION 1..37: note = polypeptide that can be used to activate T cells
ARGPESRLLEFYLAMPFATPMEAELQNQSLAQDAPPL

SEQ ID NO: 314 - protein, 35 aa, synthetic construct
  REGION 1..35: note = polypeptide that can be used to activate T cells
RGPESRLLEFYLAMPFATPMEAELARRSLAQDAPP

SEQ ID NO: 315 - protein, 31 aa, synthetic construct
  REGION 1..31: note = polypeptide that can be used to activate T cells
VPGVLLKEFTVSGNILTIRLTAADHRQLQLS

SEQ ID NO: 316 - protein, 27 aa, synthetic construct
  REGION 1..27: note = polypeptide that can be used to activate T cells
PGVLLKEFTVSGNILTIRLTAADHRQL

SEQ ID NO: 317 - protein, 29 aa, synthetic construct
  REGION 1..29: note = polypeptide that can be used to activate T cells
VPGVLLKEFTVSGNILTIRALTWYDHRQL

SEQ ID NO: 318 - protein, 28 aa, synthetic construct
  REGION 1..28: note = polypeptide that can be used to activate T cells
VPGVLLKEFTVSGNILTRRLTAADHRQL

SEQ ID NO: 319 - protein, 28 aa, synthetic construct
  REGION 1..28: note = polypeptide that can be used to activate T cells
PGVLLKEFTVSGNILTIRLTAADHRQLE

SEQ ID NO: 320 - protein, 28 aa, synthetic construct
  REGION 1..28: note = polypeptide that can be used to activate T cells
VPGVLLKEFTVSGNILTIRLTKMDHRQL

SEQ ID NO: 321 - protein, 28 aa, synthetic construct
  REGION 1..28: note = polypeptide that can be used to activate T cells
VPGVLLKEFTVSGNILTWRLTAADHRQL

SEQ ID NO: 322 - protein, 28 aa, synthetic construct
  REGION 1..28: note = polypeptide that can be used to activate T cells
VPGVLLKEFTVSGNILTWQLTMADHRQL

SEQ ID NO: 323 - protein, 30 aa, synthetic construct
  REGION 1..30: note = polypeptide that can be used to activate T cells
VPGVLLKEFTVSGNILTIRLTAADHRQLDA

SEQ ID NO: 324 - protein, 28 aa, synthetic construct
  REGION 1..28: note = polypeptide that can be used to activate T cells
VPGVLLKEFTVSGNILTIRLTSTDHRQL

SEQ ID NO: 325 - protein, 28 aa, synthetic construct
  REGION 1..28: note = polypeptide that can be used to activate T cells
RSASETSEKRPFMCAYPGCNKRYFKLSE

SEQ ID NO: 326 - protein, 28 aa, synthetic construct
  REGION 1..28: note = polypeptide that can be used to activate T cells
SETSEKRPFMYAYPGCNKRYFLSHLQMH

SEQ ID NO: 327 - protein, 32 aa, synthetic construct
  REGION 1..32: note = polypeptide that can be used to activate T cells
RSASETSNKRPFMCAYPGCNKRYFKLSHLQMH

SEQ ID NO: 328 - protein, 32 aa, synthetic construct
  REGION 1..32: note = polypeptide that can be used to activate T cells
RSASETSYQRPFMCAYPGCNKRYFKLSHLQMH

SEQ ID NO: 329 - protein, 32 aa, synthetic construct
  REGION 1..32: note = polypeptide that can be used to activate T cells
RSASETSFQRPFMCAYPGCNKRYFKLSHLQMH

SEQ ID NO: 330 - protein, 32 aa, synthetic construct
  REGION 1..32: note = polypeptide that can be used to activate T cells
RSASETLEKRPFMCAYPGCNKRYFLLSHLQMH

SEQ ID NO: 331 - protein, 32 aa, synthetic construct
  REGION 1..32: note = polypeptide that can be used to activate T cells
RSASWTSEKRPFMCAYPGCNKRYFKLNHSFKH

SEQ ID NO: 332 - protein, 32 aa, synthetic construct
  REGION 1..32: note = polypeptide that can be used to activate T cells
RSASSTSEKRPFFCAYPGCNKRYFKLSHLQMH

SEQ ID NO: 333 - protein, 32 aa, synthetic construct
  REGION 1..32: note = polypeptide that can be used to activate T cells
RSASETYSVRPFMCAYPGCNKRYFKLSHLQMH

SEQ ID NO: 334 - protein, 32 aa, synthetic construct
  REGION 1..32: note = polypeptide that can be used to activate T cells
RSASETSIKRPFYCAYPGCNKRYFKLSHLQMH

SEQ ID NO: 335 - protein, 34 aa, synthetic construct
  REGION 1..34: note = polypeptide that can be used to activate T cells
VHCCLYFIPATGHSLRPLDIEFMKRLSKVVNIVP

SEQ ID NO: 336 - protein, 37 aa, synthetic construct
  REGION 1..37: note = polypeptide that can be used to activate T cells
DTRVHCCLYFIPDTRGSLRPLDIEFMKRLSKVVNIVP

SEQ ID NO: 337 - protein, 36 aa, synthetic construct
  REGION 1..36: note = polypeptide that can be used to activate T cells
DTRVHCCLYFIPATGHSLRPLDIEFMKRLSKVVNIV

SEQ ID NO: 338 - protein, 32 aa, synthetic construct
  REGION 1..32: note = polypeptide that can be used to activate T cells
DTRVHCCLYFIPATGHSLRPLDIEFMKRLSKV

SEQ ID NO: 339 - protein, 37 aa, synthetic construct
  REGION 1..37: note = polypeptide that can be used to activate T cells
DTRVHCCLYFIPATGHSLIPLDILFMKRLSNVVNIVP

SEQ ID NO: 340 - protein, 37 aa, synthetic construct
  REGION 1..37: note = polypeptide that can be used to activate T cells
DTRVHKWLYFIPATGHSLRPLDIEFMKRLSKVVNIVP

SEQ ID NO: 341 - protein, 37 aa, synthetic construct
  REGION 1..37: note = polypeptide that can be used to activate T cells
DTRVHCLLYFIPATGHSLRPLDIEFMKRLSKVVNIVP

SEQ ID NO: 342 - protein, 37 aa, synthetic construct
  REGION 1..37: note = polypeptide that can be used to activate T cells
DTRVHCCLYFIPVTRHSLRPLDINFMKRLSKVVNIVP

SEQ ID NO: 343 - protein, 37 aa, synthetic construct
  REGION 1..37: note = polypeptide that can be used to activate T cells
DTRVHCCLYFIPATGHSLRPLDIHFMKRLSKVMNIVP

SEQ ID NO: 344 - protein, 37 aa, synthetic construct
  REGION 1..37: note = polypeptide that can be used to activate T cells
DTRVHCCLYFIPATSASLNPLDIEFMKRLSTVVNIVP

SEQ ID NO: 345 - protein, 30 aa, synthetic construct
  REGION 1..30: note = polypeptide that can be used to activate T cells
RLVNEKFREMIPFAVVGSDHEYQVNGKRIL

SEQ ID NO: 346 - protein, 26 aa, synthetic construct
  REGION 1..26: note = polypeptide that can be used to activate T cells
LVNEKFREMYPFNVVGSDHEYQVNGK

SEQ ID NO: 347 - protein, 29 aa, synthetic construct
  REGION 1..29: note = polypeptide that can be used to activate T cells
LVNEKFREMIPFAVVGSDHWYQVNGKRIL

SEQ ID NO: 348 - protein, 29 aa, synthetic construct
  REGION 1..29: note = polypeptide that can be used to activate T cells
LVNEKFRLAIPFAVVGSDHEYQVNGKRIL

SEQ ID NO: 349 - protein, 29 aa, synthetic construct
  REGION 1..29: note = polypeptide that can be used to activate T cells
LVNEKFREMIPFQVVGSDHNYQVNGKRIL

SEQ ID NO: 350 - protein, 29 aa, synthetic construct
  REGION 1..29: note = polypeptide that can be used to activate T cells
LVNEKFREMIPFAVVGSTHYYQVNGKRIL

SEQ ID NO: 351 - protein, 30 aa, synthetic construct
  REGION 1..30: note = polypeptide that can be used to activate T cells
RLVNPKFREMIPFAVVGSDHNYQVNGKRIL

SEQ ID NO: 352 - protein, 29 aa, synthetic construct
  REGION 1..29: note = polypeptide that can be used to activate T cells
LVNWKFRFMIPFAVVGSDHEYQVNGKQIL

SEQ ID NO: 353 - protein, 29 aa, synthetic construct
  REGION 1..29: note = polypeptide that can be used to activate T cells
LVNEKFREMIPFAVVGSLHIYQVNMKRIL

SEQ ID NO: 354 - protein, 29 aa, synthetic construct
  REGION 1..29: note = polypeptide that can be used to activate T cells
LVNEKFREMIPFAVVGSTHVYQVNGKRIL

SEQ ID NO: 355 - protein, 29 aa, synthetic construct
  REGION 1..29: note = polypeptide that can be used to activate T cells
FAYLRDLLIRTHMQNIKDITSSIHFEAYR

SEQ ID NO: 356 - protein, 30 aa, synthetic construct
  REGION 1..30: note = polypeptide that can be used to activate T cells
TTHCEFAYLRDLLIRTHMQNIKDITSSIHF

SEQ ID NO: 357 - protein, 34 aa, synthetic construct
  REGION 1..34: note = polypeptide that can be used to activate T cells
TTHWNFAYLRDLLIRTHMQNIKDITYTIHFEAYR

SEQ ID NO: 358 - protein, 34 aa, synthetic construct
  REGION 1..34: note = polypeptide that can be used to activate T cells
TTHCEFAYLRDLLIRTHMQNINDITLSIHFEAYE

SEQ ID NO: 359 - protein, 34 aa, synthetic construct
  REGION 1..34: note = polypeptide that can be used to activate T cells
TTHCEFAYLRDLLIRTHMQNIKDITYYIHFEAYA

SEQ ID NO: 360 - protein, 34 aa, synthetic construct
  REGION 1..34: note = polypeptide that can be used to activate T cells
TTHVWFAYLRDLLIRTHMQNIKDITSSIHFEAYR

SEQ ID NO: 361 - protein, 34 aa, synthetic construct
  REGION 1..34: note = polypeptide that can be used to activate T cells
TTHCEFAYLRLLLIRTHMQNIKDITSSIHFEAYR

SEQ ID NO: 362 - protein, 34 aa, synthetic construct
  REGION 1..34: note = polypeptide that can be used to activate T cells
TTHPEFAYLRDLLIRTHMQNIKDITSSIHFEAYR

SEQ ID NO: 363 - protein, 29 aa, synthetic construct
  REGION 1..29: note = polypeptide that can be used to activate T cells
FAYLRDLLIRTHMQNIKDITSSIHFEAYR

SEQ ID NO: 364 - protein, 34 aa, synthetic construct
  REGION 1..34: note = polypeptide that can be used to activate T cells
THCEFAYLRDLLIVVQHMQNIKDITSSIHFEAYR

SEQ ID NO: 365 - protein, 189 aa, Homo sapiens
MTDKTEKVAVDPETVFKRPRECDSPSYQKRQRMALLARKQGAGDSLIAGSAMSKAKKLMTGHAIPPSQLDSQIDDFTGFSKDRMMQKPGSNAPVGGNVTSSFSGDDLECRETASSPKSQREINADIKRKLVKELRCVGQKYEKIFEMLEGVQGPTAVRKRFFESIIKEAARCMRRDFVKHLKKKLKRMI